SEQUENCE LISTING (7 sequences)

SEQ ID NO: 1 - protein, 433 aa, Mus musculus
  PEPTIDE (1)..(433): Cyclin B1
MALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKV

SEQ ID NO: 2 - protein, 294 aa, Mus musculus
  PEPTIDE (1)..(294): RARRES1 isoform 1
MQPRRQRLPAPWSGPRGPRPTAPLLALLLLLAPVAAPAGSGDPDDPGQPQDAGVPRRLLQQAARAALHFFNFRSGSPSALRVLAEVQEGRAWINPKEGCKVHVVFSTERYNPESLLQEGEGRLGKCSARVFFKNQKPRPTINVTCTRLIEKKKRQQEDYLLYKQMKQLKNPLEIVSIPDNHGHIDPSLRLIWDLAFLGSSYVMWEMTTQVSHYYLAQLTSVRQWKTNDDTIDFDYTVLLHELSTQEIIPCRIHLVWYPGKPLKVKYHCQELQTPEEASGTEEGSAVVPTELSNF

SEQ ID NO: 3 - protein, 228 aa, Mus musculus
  PEPTIDE (1)..(228): RARRES1 isoform 2
MQPRRQRLPAPWSGPRGPRPTAPLLALLLLLAPVAAPAGSGDPDDPGQPQDAGVPRRLLQQAARAALHFFNFRSGSPSALRVLAEVQEGRAWINPKEGCKVHVVFSTERYNPESLLQEGEGRLGKCSARVFFKNQKPRPTINVTCTRLIEKKKRQQEDYLLYKQMKQLKNPLEIVSIPDNHGHIDPSLRLIWDLAFLGSSYVMWEMTTQVSHYYLAQLTSVRQWVRKT

SEQ ID NO: 4 - RNA, 1545 nt, Mus musculus
  mRNA (1)..(1545): RARRES1 isoform 1
uuuccgcgagcgccggcacugcccgcuccgagcccgugucugucgggugccgagccaacuuuccugcguccaugcagccccgccggcaacggcugccugcucccugguccgggcccaggggcccgcgccccaccgccccgcugcucgcgcugcugcuguugcucgccccgguggcggcgcccgcgggguccggggaccccgacgacccugggcagccucaggaugcuggggucccgcgcaggcuccugcagcaggcggcgcgcgcggcgcuucacuucuucaacuuccgguccggcucgcccagcgcgcuacgagugcuggccgaggugcaggagggccgcgcguggauuaauccaaaagagggauguaaaguucacguggucuucagcacagagcgcuacaacccagagucuuuacuucaggaaggugagggacguuuggggaaauguucugcucgaguguuuuucaagaaucagaaacccagaccaaccaucaauguaacuuguacacggcucaucgagaaaaagaaaagacaacaagaggauuaccugcuuuacaagcaaaugaagcaacugaaaaaccccuuggaaauagucagcauaccugauaaucauggacauauugaucccucucugagacucaucugggauuuggcuuuccuuggaagcucuuacgugaugugggaaaugacaacacaggugucacacuacuacuuggcacagcucacuagugugaggcaguggaaaacuaaugaugauacaauugauuuugauuauacuguucuacuucaugaauuaucaacacaggaaauaauucccugucgcauucacuuggucugguacccuggcaaaccucuuaaagugaaguaccacugucaagagcuacagacaccagaagaagccuccggaacugaagaaggaucagcuguaguaccaacagagcuuaguaauuucuaaaaagaaaaaaugaucuuuuuccgacuucuaaacaagugacuauacuagcauaaaucauucuucuaguaaaacagcuaagguauagacauucuaauaauuugggaaaaccuaugauuacaaguaaaaacucagaaaugcaaagauguugguuuuuuguuucucagucugcuuuagcuuuuaacucuggaagcgcaugcacacugaacucugcucagugcuaaacagucaccagcagguuccucaggguuucagcccuaaaauguaaaaccuggauaaucaguguauguugcaccagaaucagcauuuuuuuuuuaacugcaaaaaaugauggucucaucucugaauuuauauuucucauucuuuugaacauacuauagcuaauauauuuuauguugcuaaauugcuucuaucuagcauguuaaacaaagauaauauacuuucgaugaaaguaaauuauaggaaaaaaauuaacuguuuuaaaaagaacuugauuauguuuuaugauuucaggcaaguauucauuuuuaacuugcuaccuacuuuuaaauaaauguuuacauuucuaaauaaaaaaaaaaaaaaaaaaaaa

SEQ ID NO: 5 - RNA, 883 nt, Mus musculus
  mRNA (1)..(883): RARRES1 isoform 2
uuuccgcgagcgccggcacugcccgcuccgagcccgugucugucgggugccgagccaacuuuccugcguccaugcagccccgccggcaacggcugccugcucccugguccgggcccaggggcccgcgccccaccgccccgcugcucgcgcugcugcuguugcucgccccgguggcggcgcccgcgggguccggggaccccgacgacccugggcagccucaggaugcuggggucccgcgcaggcuccugcagcaggcggcgcgcgcggcgcuucacuucuucaacuuccgguccggcucgcccagcgcgcuacgagugcuggccgaggugcaggagggccgcgcguggauuaauccaaaagagggauguaaaguucacguggucuucagcacagagcgcuacaacccagagucuuuacuucaggaaggugagggacguuuggggaaauguucugcucgaguguuuuucaagaaucagaaacccagaccaaccaucaauguaacuuguacacggcucaucgagaaaaagaaaagacaacaagaggauuaccugcuuuacaagcaaaugaagcaacugaaaaaccccuuggaaauagucagcauaccugauaaucauggacauauugaucccucucugagacucaucugggauuuggcuuuccuuggaagcucuuacgugaugugggaaaugacaacacaggugucacacuacuacuuggcacagcucacuagugugaggcaguggguaagaaaaaccugaaaauuaacuugugccacaagaguuacaaucaaaguggucuccuuagacugaauucaugcgaacuucuaauuucauaucaagaguuguaaucacauuuauuucaauaaauaugugaguuccugcaa

SEQ ID NO: 6 - protein, 44 aa, Mus musculus
  SITE (1)..(44): 251˜294 peptides derived from RARRES1 isoform 1
RIHLVWYPGKPLKVKYHCQELQTPEEASGTEEGSAVVPTELSNF

SEQ ID NO: 7 - RNA, 183 nt, Mus musculus
  mRNA (1)..(183): 771˜953 mRNA derived from RARRES1 isoform 1 full length
gauuauacuguucuacuucaugaauuaucaacacaggaaauaauucccugucgcauucacuuggucugguacccuggcaaaccucuuaaagugaaguaccacugucaagagcuacagacaccagaagaagccuccggaacugaagaaggaucagcuguaguaccaacagagcuuaguaauuuc

(19) United States
(12) Patent Application Publication
(10) Pub. No.: US 2022/0026415 A1
(43) Pub. Date: Jan. 27, 2022

(54) A METHOD FOR SCREENING A THERAPEUTIC AGENT FOR CANCER USING BINDING INHIBITOR OF CYCLIN-DEPENDENT KINASE 1 (CDK1)-CYCLIN B1 AND RETINOIC ACID RECEPTOR RESPONDER 1 (RARRES1) GENE KNOCKOUT ANIMAL MODEL

(71) Applicant: NATIONAL CANCER CENTER, Goyang-si (KR)

(72) Inventors: Kyungtae KIM, Seoul (KR); Hyoun Sook KIM, Goyang-si (KR); Charny PARK, Goyang-si (KR); Doyeong YU, Uiwang-si (KR); Eun-Kyung YOON, Goyang-si (KR); Su-Hyung LEE, Gunpo-si (KR); Ho LEE, Goyang-si (KR)

(73) Assignee: NATIONAL CANCER CENTER, Goyang-si (KR)

(21) Appl. No.: 17/296,922

(22) PCT Filed: Dec. 31, 2018
(86) PCT No.: PCT/KR2018/016994

§ 371 Date: May 25, 2021

(30) Foreign Application Priority Data
Nov. 26, 2018 (KR) 10-2018-0147664
Dec. 3, 2018 (KR) 10-2018-0153686

(51) Int. Cl. G01N 33/50; A01K 67/027; C12N 15/85

(52) U.S. Cl. CPC G01N 33/5088; A01K 67/0276; C12N 15/8509; A01K 2217/206; A01K 2227/105; G01N 2333/912; A01K 2267/0331; C12N 2800/30; C12N 2015/8572; G01N 2333/4739; A01K 2217/075

(57) ABSTRACT

The present invention relates to a method of screening for a cancer therapeutic agent using Cyclin B1, Cyclin-dependent kinase 1 (CDK1), and retinoic acid receptor responder 1 (RARRES1), and a composition for diagnosing cancer or predicting a prognosis using the same. As a result of having conducted intensive studies to discover molecular mechanisms for diagnosing cancer and predicting a prognosis, the inventors of the present invention confirmed that in cancer-derived samples, according to the degree of mutual binding between RARRES1 and CDK1 or Cyclin B1, the mitosis of cancer cells was arrested, the formation of CDK1-Cyclin B1 complexes was suppressed, and the degradation of these proteins was promoted, and thus RARRES1 was a crucial factor in the diagnosis of cancer, prognosis prediction, and the treatment of cancer. In addition, through these findings, it is anticipated that RARRES1 may be widely used in screening for a cancer therapeutic agent exhibiting a decrease in the degree of binding between CDK1 and Cyclin B1, an increase in the degree of binding between the RARRES1 gene and CDK1 or Cyclin B1, and a decrease in an amount or activity of the CDK1 protein or the Cyclin B1 protein, and in the development of drugs. In addition, the present invention relates to a targeting vector including a portion of the Rarres1 gene and sequences used in producing a conditional knockout animal model, an animal cell for producing a tumorigenic animal model, which is produced using the targeting vector, a tumorigenic Rarres1^{-/-} animal model produced using the animal cell, a method of producing the animal model, and a method of screening for a cancer therapeutic agent by using the method. Thus, as a result of having conducted intensive studies to discover molecular mechanisms for diagnosing cancer and predicting a prognosis, the inventors of the present invention confirmed that a Rarres1^{-/-} animal model was prone to spontaneous tumors and exhibited increased phosphorylation of CDK1 and Cyclin B1 and a high activity of a CDK1-Cyclin B1 complex, and thus it was confirmed that the tumor cell cycle progression was unusually rapid due to a decrease in protein degradation ability. In particular, it was confirmed that stem cell proliferation was increased, and chromosomes were unstable upon induction of mitotic defects and mitosis, from which it was confirmed that RARRES1 is a crucial factor in diagnosing cancer, predicting a prognosis, and treating cancer. Moreover, it is anticipated that the Rarres1^{-/-} animal model can be variously used for screening for a cancer therapeutic agent and developing a drug, through the relationship between RARRES1 and a CDK1-Cyclin B1 complex, the quantitative regulation of the CDK1 and Cyclin B proteins, and an increase in stem cell proliferative ability.

### TECHNICAL FIELD

The present invention relates to a method of screening for a cancer therapeutic agent by using a binding inhibitor of CDK1-Cyclin B1, and a composition for diagnosing cancer or predicting a prognosis, and more particularly, to a method of screening for a therapeutic agent that reduces a binding level of CDK1 and Cyclin B1, increases a binding level of RARRES1 and CDK1 or Cyclin B1, and reduces the amount or activity of the CDK1 or Cyclin B1 protein, a composition for diagnosing cancer or predicting a prognosis, and a pharmaceutical composition for preventing or treating cancer.

In addition, the present invention relates to a RARRES1 gene knockout animal model, and more particularly, to a targeting vector including a portion of the RARRES1 gene and sequences used in producing a conditional knockout animal model, an animal cell for producing a tumorigenic animal model, which is produced using the targeting vector, a Rarres1^{-/-} animal model produced using the animal cell, a method of producing the animal model, and a method of screening for a cancer therapeutic agent by using the animal model.

### BACKGROUND ART

The term “tumor” refers to a mass abnormally grown by autonomous overgrowth of body tissues, and can be classified into a benign tumor and a malignant tumor. Benign tumors grow relatively slowly and do not metastisize, whereas malignant tumors grow rapidly while infiltrating into the surrounding tissues, and spread or transit to each part of the body, and thus are life-threatening. Therefore, malignant tumors may be regarded, in the same sense, as cancer. Cells, which are the smallest unit of the body, normally divide and grow by the regulatory function of cells themselves, and when the lifespan of cells ends or cells get damaged, they themselves die and thus maintain a balance in an overall number of cells. However, when cells have a problem with such a regulatory function thereof, due to various causes, abnormal cells, which would normally die, excessively proliferate, and in some cases, infiltrate into the surrounding tissues and organs to thereby form a mass, resulting in destruction or modification of existing structures, and this condition may be defined as cancer.

To diagnose such cancer, an X-ray examination is often used for lung cancer or myeloma to acquire its shaded image by simple radiography, tomography, and the like, and visceral cancer is diagnosed by observing its various shaded images using a contrast medium. Especially in stomach cancer, colorectal cancer, and the like, the state of the mucous membrane is checked using a double contrast method to detect even fine lesions such as early stomach cancer or colorectal cancer, and thus this method is very helpful for early detection. For endoscopy, a rigid endoscope has long been used for visceral examination, but has a limited range of visibility, and thus this method is not very helpful for diagnosis. Thus, only an S colonoscope is currently used. The development of a flexible fiberscope, which can be bent freely, plays a major role in diagnosis, especially early diagnosis of various visceral cancers. Endoscopic biopsy is also available and provides good clues to definite diagnosis, and can be used for group examination in addition to general hospital examination, and thus is more usefully used in stomach cancer, and the like. Examples of endoscopes which are currently widely used in clinical trials include a flexible gastrofiberscope, a flexible colonofiberscope, and a sigmoidoscope, and a peritoneoscope, a mediastino scope, a bronchoscope, or the like is also used. Since G. N. Papanicolaou discovered the characteristics of malignant tumor cells in smears of cervical secretions through cell diagnosis, it has brought dramatic advances in group screening and diagnosis, especially early diagnosis of cervical cancer. In addition to cervical cancer, it is currently used for secretions of the stomach, the lungs, the prostate, the breast, the urinary tract, the pancreas, and the like, and cell diagnosis of the thyroid, the breast, and the like by centesis is also widely used. Cancer is diagnosed by histopathologic microscopy of tissue pieces obtained through biopsy. Such tissue pieces are obtained using an endoscopic method or obtained from the breast, the vagina, or the like while performing an operation. Although cancer can be diagnosed through these diagnosis methods, generally, there are many cases in which metastasis to the surrounding tissues or remote metastasis has already occurred when cancer is first diagnosed, and since the survival rate and prognosis of patients with cancer are worse as cancer is detected later, early diagnosis is very important. Therefore, understanding of the onset and progression of cancer is very important, but studies on the molecular mechanism inducing the onset of cancer have not been adequately conducted.

Meanwhile, CDK1 is a major cell cycle regulator. In yeast, cell cycle progression is controlled by a single CDK, known as Cdc28 of Saccharomyces cerevisiae and Cdc2 of Schizosaccharomyces pombe, and this binds to specific Cyclins at different stages of the cell cycle. By genetic studies on mice, the systematic knockout of Cdks of the mouse germline has shown that Cdk2, Cdk4, and Cdk6 are not essential for the cell cycle of most cell types. Only elimination of Cdk1 causes cell cycle arrest and embryonic lethality at the two-cell stage. CDK1 activity controls both M phase entry and exit. In G2/M transition, CDK1-Cyclin B1 activation leads to phosphorylation of various proteins that control chromosome condensation, nuclear envelope breakdown, and spindle assembly. At the onset of anaphase, CDK1-Cyclin B1 is involved in this event by controlling the activity of separase, which is a protease that cleaves cohesion complexes that hold sister chromatids together. The regulation of CDK1 activity is controlled at multiple levels, such as binding with its regulatory subunits (Cyclin A and B), interactions with Cyclin-dependent kinase inhibitors (CKIs), and phosphorylation and dephosphorylation of specific residues by the activating kinase CAK (CDK activating kinase) or by several inhibitor kinases including Wee1 and Myt1 or phosphatase Cdc25. Of the several proteins associated with the progression of tumors, mice over-expressed Cyclin B1, which is a regulatory subunit of CDK1 in mitosis, exhibited a high tumor incidence.

In addition, retinoic acid receptor responder 1 (RARRES1) was initially identified as the most upregulated gene in skin raft culture by tazarotene, which is a synthetic retinoid. This gene is found only in vertebrates and is an evolutionally conserved gene among the species. In humans, it is localized at q arm 25.32 loci of chromosome 3. Alternatively spliced transcript variants that encode two distinct isoforms exist. Isoform 1 (RARRES1-1) consists of six exons and encodes 294 proteins, and isoform 2 (RARRES1-2) consists of five exons and encodes 228 proteins. The C-terminal and 3′-untranslated region (UTR) were different forms between these two isoforms. The expression of RARRES1 in a variety of tumor tissues and cell lines, including prostate cancer, breast cancer, lung cancer, liver cancer, colon cancer, gastric cancer, esophagus cancer, nasopharyngeal cancer, endometrium cancer, and head and neck cancer is frequently lost or silenced, mostly due to hypermethylation of its promoter region.

However, the association of CDK1-Cyclin B1 and RARRES1 with the diagnosis of cancer, the onset of cancer cells, and the progression of cancer has not yet been known.

In addition, term “tumor” refers to a mass abnormally grown by autonomous growth of body tissues, and can be classified into a benign tumor and a malignant tumor. Benign tumors grow relatively slowly and do not metastisize, whereas malignant tumors grow rapidly while infiltrating into the surrounding tissues, and spread or transit to each part of the body, and thus are life-threatening. Therefore, malignant tumors may be regarded, in the same sense, as cancer. Cells, which are the smallest unit of the body, normally divide and grow by the regulatory function of cells themselves, and when the lifespan of cells ends or cells get damaged, they themselves die and thus maintain a balance in an overall number of cells. However, when cells have a problem with such a regulatory function thereof, due to various causes, abnormal cells, which would normally die, excessively proliferate, and in some cases, infiltrate into the surrounding tissues and organs to thereby form a mass, resulting in destruction or modification of existing structures, and this condition may be defined as cancer.

To diagnose such cancer, an X-ray examination is often used for lung cancer or myeloma to acquire its shaded image by simple radiography, tomography, and the like, and visceral cancer is diagnosed by observing its various shaded images using a contrast medium. Especially in stomach cancer, colorectal cancer, and the like, the state of the mucous membrane is checked using a double contrast method to detect even fine lesions such as early stomach cancer or colorectal cancer, and thus this method is very helpful for early detection. For endoscopy, a rigid endoscope has long been used for visceral examination, but has a limited range of visibility, and thus this method is not very helpful for diagnosis. Thus, only an S colonoscope is currently used. The development of a flexible fiberscope, which can be bent freely, plays a major role in diagnosis, especially early diagnosis of various visceral cancers. Endoscopic biopsy is also available and provides good clues to definite diagnosis, and can be used for group examination in addition to general hospital examination, and thus is more usefully used in stomach cancer, and the like. Examples of endoscopes which are currently widely used in clinical practice include a flexible gastrofiberscope, a flexible colonofiberscope, and a sigmoidoscope, and a peritoneoscope, a mediastino scope, a bronchoscope, or the like is also used. Since G. N. Papanicolaou discovered the characteristics of malignant tumor cells in smears of cervical secretions through cell diagnosis, it has brought dramatic advances in group screening and diagnosis, especially early diagnosis of cervical cancer. In addition to cervical cancer, it is currently used for secretions of the stomach, the lungs, the prostate, the breast, the urinary tract, the pancreas, and the like, and cell diagnosis of the thyroid, the breast, and the like by centesis is also widely used. Cancer is diagnosed by histopathologic microscopy of tissue pieces obtained through biopsy. Such tissue pieces are obtained using an endoscopic method or obtained from the breast, the vagina, or the like while performing an operation. Although cancer can be diagnosed through these diagnosis methods, generally, there are many cases in which metastasis to the surrounding tissues or remote metastasis has already occurred when cancer is first diagnosed, and since the survival rate and prognosis of patients with cancer are worse as cancer is detected later, early diagnosis is very important. Therefore, understanding of the onset and progression of cancer is very important, but studies on the molecular mechanism inducing the onset of cancer and effective diagnosis are insufficient.

Meanwhile, the retinoic acid receptor responder 1 (RARRES1) gene was initially identified as the most upregulated gene in skin raft culture by tazarotene, which is a synthetic retinoid. This gene is found only in vertebrates and is an evolutionally conserved gene among the species. In humans, it is localized at q arm 25.32 loci of chromosome 3. Spliced transcript variants (alternatively spliced transcript variants) that encode two distinct isoforms exist. Isoform 1 (RARRES1-1) consists of six exons and encodes 294 proteins, and isoform 2 (RARRES1-2) consists of five exons and encodes 228 proteins. The C-terminal and 3′-untranslated region (UTR) were different forms between these two isoforms. The expression of RARRES1 in a variety of tumor tissues and cell lines, including prostate cancer, breast cancer, lung cancer, liver cancer, colon cancer, gastric cancer, esophagus cancer, nasopharyngeal cancer, endometrium cancer, and head and neck cancer is frequently lost or silenced, mostly due to hypermethylation of its promoter region(KR2013-0069924, KR10-1348852).

Therefore, there is a need for effective diagnosis and treatment of cancer, and it is necessary to establish animal models suitable for the development of biological samples needed for studies on mechanisms and treatment of cancer. However, studies on the effect of RARRES1 on the diagnosis of cancer, the onset of cancer cells, and the progression of cancer, and studies on related animal models are still insufficient.

### DISCLOSURE OF INVENTION

### Technical Problem

As a result of having conducted intensive studies to discover molecular mechanisms for diagnosing cancer and predicting a prognosis, the inventors of the present invention confirmed that in cancer-derived samples, according to the degree of mutual binding between retinoic acid receptor responder 1 (RARRES1) and Cyclin-dependent kinase 1 (CDK1) or Cyclin B1, the mitosis of cancer cells was arrested, the formation of CDK1-Cyclin B1 complexes was suppressed, and the degradation of these proteins was promoted, and thus they were crucial factors in the diagnosis of cancer, prognosis prediction, and the treatment of cancer, and thus completed the present invention based on these findings.

Therefore, the present invention provides a method of screening for a cancer therapeutic agent, including: (a) treating a sample with candidate materials in vitro; (b) measuring a degree of binding between CDK1 and Cyclin B1 of the sample or measuring an amount or activity of the CDK1 protein or the Cyclin B1 protein; and (c) selecting, as a cancer therapeutic agent, a candidate material exhibiting a decrease in the degree of binding between CDK1 and Cyclin B1, or a candidate material exhibiting a decrease in the amount or activity of the CDK1 protein or the Cyclin B1 protein, as compared to that in a group not treated with the candidate materials.

The present invention also provides a composition for diagnosing cancer or predicting a prognosis, which includes an agent for measuring an mRNA level of RARRES1 or a level of a peptide encoded by the RARRES1 gene.

The present invention also provides a kit for diagnosing cancer or predicting a prognosis, which includes the composition for diagnosing cancer or predicting a prognosis.

The present invention also provides a pharmaceutical composition for preventing or treating cancer, which includes, as an active ingredient, an inhibitor of binding between CDK1 and Cyclin B1.

The present invention also provides a tumorigenic Rarres1^{+/N} chimeric animal model produced by injecting, into a blastocyst, an animal cell for producing a tumorigenic animal model, which is transfected with a retinoic acid receptor responder 1 (Rarres1) targeting vector for producing a tumorigenic animal model, the targeting vector including a DNA sequence consisting of, in the following order, a first locus of X-over P1 (loxP) site; a drug resistance gene region; a gene fragment including exon 3 of the Rarres1 genomic gene; and a second loxP site.

The present invention also provides a tumorigenic Rarres1^{+/-} animal model produced by crossing the Rarres1^{+/N} chimeric animal model with an animal expressing Cre recombinase.

The present invention also provides a method of producing a tumorigenic Rarres1^{-/-} animal model, including: (a) producing the Rarres1^{+/N} chimeric animal model; (b) producing a Rarres1^{+/-} animal model through crossing of the chimeric animal model with an animal expressing Cre recombinase; and (c) selecting a Rarres1^{-/-} animal model from among progenies obtained by crossing the Rarres1^{+/-} animal model of process (b).

The present invention also provides a tumorigenic Rarres1^{-/-} animal model produced by the above-described production method.

The present invention also provides a method of screening for a tumor therapeutic agent, including: (a) treating a sample of a tumorigenic Rarres1^{-/-} animal model with candidate materials; (b) measuring phosphorylation levels of Cyclin-dependent kinase 1 (CDK1) and Cyclin B1 of the sample, measuring amounts and activities of the CDK1 protein and the Cyclin B1 protein, measuring the expression or activity of muscle, intestine and stomach expression 1(Mist1) and leucine-rich repeat-containing G-protein coupled receptor 5 (LGR5), or measuring the activity of surfactant protein C (SPC)-positive cells; and selecting, as a tumor therapeutic agent, a candidate material exhibiting a decrease in phosphorylation levels of CDK1 and Cyclin B1, a candidate material exhibiting a decrease in amounts or activities of the CDK1 protein and the Cyclin B1 protein, a candidate material exhibiting a decrease in expression or activity of muscle, intestine and stomach expression 1 (Mist1) and leucine-rich repeat-containing G-protein coupled receptor 5 (LGR5), or a candidate material exhibiting a decrease in activity of SPC-positive cells, as compared to that in a group not treated with the candidate materials.

However, technical problems to be solved by the present invention are not limited to the above-described technical problems, and other unmentioned technical problems will become apparent from the following description to those of ordinary skill in the art.

### Solution to Problem

To achieve the above-described objects of the present invention, the present invention provides a method of screening for a cancer therapeutic agent, including: (a) treating a sample with candidate materials in vitro; (b) measuring a degree of binding between CDK1 and Cyclin B1 of the sample or measuring an amount or activity of the CDK1 protein or the Cyclin B1 protein; and (c) selecting, as a cancer therapeutic agent, a candidate material exhibiting a decrease in the degree of binding between CDK1 and Cyclin B1, or a candidate material exhibiting a decrease in the amount or activity of the CDK1 protein or the Cyclin B1 protein, as compared to that in a group not treated with the candidate materials.

In one embodiment of the present invention, the method may further include, in the process (b), measuring a degree of binding between retinoic acid receptor responder 1 (RARRES1) and CDK1 or Cyclin B1 of the sample; and, in the process (c), selecting, as a cancer therapeutic agent, a candidate material exhibiting a decrease in the degree of binding between CDK1 and Cyclin B1 and an increase in the degree of binding between RARRES1 and CDK1 or Cyclin B1.

In another embodiment of the present invention, in the process (c), the decrease in the amount or activity of the CDK1 protein may be an increase in the degradation of CDK1 in lysosomes due to an increased degree of binding between RARRES1 and CDK1.

In another embodiment of the present invention, the sample may be derived from one or more selected from the group consisting of prostate cancer, lung cancer, and breast cancer patients.

In another embodiment of the present invention, the decrease in in the degree of binding between CDK1 and Cyclin B1 may be inhibition of phosphorylation of serine 126 of the Cyclin B1 protein.

In another embodiment of the present invention, the Cyclin B1 protein may have an amino acid sequence of SEQ ID NO: 1.

In another embodiment of the present invention, the increase in the degree of binding between RARRES1 and CDK1 may be binding to inactivated CDK1 at a C-terminal portion containing amino acids 251 to 294 of the RARRES1 protein.

In another embodiment of the present invention, the amino acids 251 to 294 of the RARRES1 protein may have an amino acid sequence of SEQ ID NO: 6.

The present invention also provides a composition for diagnosing cancer or predicting a prognosis, which includes an agent for measuring a level of mRNA of RARRES1 or a level of a peptide encoded by the RARRES1 gene.

In one embodiment of the present invention, the mRNA of the RARRES1 gene may have a base sequence of SEQ ID NO: 4 or 5, and preferably may include a nucleotide of a base sequence of SEQ ID NO: 7.

In another embodiment of the present invention, the peptide encoded by the RARRES1 gene may have an amino acid sequence of SEQ ID NO: 2 or 3, and preferably may include a peptide having an amino acid sequence of SEQ ID NO: 6.

The present invention also provides a kit for diagnosing cancer or predicting a prognosis, which includes a composition for diagnosing cancer or predicting a prognosis.

The present invention provides a pharmaceutical composition for preventing or treating cancer, which includes, as an active ingredient, an inhibitor of binding between CDK1 and Cyclin B1.

In one embodiment of the present invention, the cancer may be one or more selected from the group consisting of prostate cancer, lung cancer, and breast cancer.

The present invention also provides a method of preventing or treating cancer, which includes administering, to an individual, a pharmaceutical composition including, as an active ingredient, an inhibitor of binding between CDK1 and Cyclin B1.

The present invention also provides a use of a pharmaceutical composition for preventing or treating cancer, the pharmaceutical composition including, as an active ingredient, an inhibitor of binding between CDK1 and Cyclin B1.

The present invention also provides a tumorigenic Rarres1^{+/N} chimeric animal model produced by injecting, into a blastocyst, an animal cell for producing a tumorigenic animal model, which is transfected with a retinoic acid receptor responder 1 (Rarres1) targeting vector for producing a tumorigenic animal model, the targeting vector including a DNA sequence consisting of, in the following order, a first locus of X-over P1 (loxP) site; a drug resistance gene region; a gene fragment including exon 3 of the Rarres1 genomic gene; and a second loxP site.

In one embodiment of the present invention, the targeting vector may include, in the front of the first locus of X-over P1 (loxP) site, a DNA sequence consisting of, in the following order, a splicing acceptor (SA), β-galactosidase (β gal), and an SV40 polyA signal (pA).

In another embodiment of the present invention, the drug resistance gene region may be a neomycin resistance gene.

The present invention also provides a tumorigenic Rarres1^{+/-} animal model produced by crossing the Rarres1^{+/N} chimeric animal model with an animal expressing Cre recombinase.

In one embodiment of the present invention, a gene encoding the Cre recombinase of the animal expressing Cre recombinase may be operably linked to a Zona pellucida 3 (Zp3) promoter.

The present invention also provides a method of producing a tumorigenic Rarres1^{-/-} animal model, including: (a) producing the Rarres1^{+/N} chimeric animal model; (b) producing a Rarres1^{+/-} animal model through crossing of the chimeric animal model with an animal expressing Cre recombinase; and (c) selecting a Rarres1^{-/-} animal model from among progenies obtained by crossing the Rarres1^{+/-} animal model of process (b).

In one embodiment of the present invention, the animal may be a mouse.

In another embodiment of the present invention, the Rarres1^{-/-} animal model may have a tumor induced by deletion of Rarres1.

The present invention also provides a tumorigenic Rarres1^{-/-} animal model produced by the above-described production method.

In one embodiment of the present invention, the animal model may induce a mitotic defect or resist mitotic stress.

In another embodiment of the present invention, the animal model may induce a somatic mutation.

In another embodiment of the present invention, in the animal model, one or more genes selected from the group consisting of Ccnd1, Cdkn1a, Cdkn2A, Nanog, Psrc1, and Nup214 may be overexpressed in a mitotic cell cycle.

The present invention also provides a method of screening for a tumor therapeutic agent, including: (a) treating a sample of a tumorigenic Rarres1^{-/-} animal model with candidate materials; (b) measuring phosphorylation levels of Cyclin-dependent kinase 1 (CDK1) and Cyclin B1 of the sample, measuring amounts or activities of the CDK1 protein and the Cyclin B1 protein, measuring the expression or activity of Mist1 and LGR5, or measuring the activity of surfactant protein C (SPC)-positive cells; and selecting, as a tumor therapeutic agent, a candidate material exhibiting a decrease in phosphorylation levels of CDK1 and Cyclin B1, a candidate material exhibiting a decrease in amounts or activities of the CDK1 protein and the Cyclin B1 protein, a candidate material exhibiting a decrease in expression or activity of muscle, intestine and stomach expression 1 (Mist1) and leucine-rich repeat-containing G-protein coupled receptor 5 (LGR5), or a candidate material exhibiting a decrease in activity of SPC-positive cells, as compared to that in a group not treated with the candidate materials.

In one embodiment of the present invention, the sample may be derived

from one or more selected from the group consisting of patients with spleen cancer, thymus cancer, liver cancer, lung cancer, renal cancer, thyroid cancer, small intestine cancer, stomach cancer, uterine cancer, and myeloid leukemia.

In another embodiment of the present invention, the tumor may be one or more selected from the group consisting of spleen cancer, thymus cancer, liver cancer, lung cancer, renal cancer, thyroid cancer, small intestine cancer, stomach cancer, uterine cancer, and myeloid leukemia.

In another embodiment of the present invention, the Mist1, LGR5, or SPC may be a stem cell marker.

### Advantageous Effects of Invention

As a result of having conducted intensive studies to discover molecular mechanisms for diagnosing cancer and predicting a prognosis, the inventors of the present invention confirmed that in cancer-derived samples, according to the degree of mutual binding between retinoic acid receptor responder 1 (RARRES1) and Cyclin-dependent kinase 1 (CDK1) or Cyclin B1, the mitosis of cancer cells was arrested, the formation of CDK1-Cyclin B1 complexes was suppressed, and the degradation of these proteins was promoted, and thus RARRES1 was a crucial factor in the diagnosis of cancer, prognosis prediction, and the treatment of cancer. In addition, through these findings, it is anticipated that RARRES1 can be widely used in screening for a cancer therapeutic agent exhibiting a decrease in a degree of binding between CDK1 and Cyclin B1, an increase in a degree of binding between the RARRES1 and CDK1 or Cyclin B1, and a decrease in an amount or activity of the CDK1 protein or the Cyclin B1 protein, and in the development of drugs.

In addition, as a result of having conducted intensive studies to discover molecular mechanisms for diagnosing cancer and predicting a prognosis, the inventors of the present invention confirmed that a Rarres1^{-/-} animal model was prone to spontaneous tumors and exhibited increased phosphorylation of CDK1 and Cyclin B1 and a high activity of a CDK1-Cyclin B1 complex, and thus it was confirmed that the tumor cell cycle progression was unusually rapid due to a decrease in protein degradation ability. In particular, it was confirmed that stem cell population was increased, and chromosomes were unstable upon induction of mitotic defects and mitosis, from which it was confirmed that RARRES1 is a crucial factor in diagnosing cancer, predicting a prognosis, and treating cancer. Moreover, it is anticipated that the Rarres1^{-/-} animal model can be variously used for screening for a cancer therapeutic agent and developing a drug, through the relationship between RARRES1 and a CDK1-Cyclin B1 complex, the quantitative regulation of the CDK1 and Cyclin B proteins, and an increase in stem cell proliferative ability.

### BRIEF DESCRIPTION OF DRAWINGS

FIGS. 1A, 1B, and 1C illustrate expression patterns of RARRES1 in a prostate cancer cell line (1A), a lung cancer cell line (1B), and a breast cancer cell line (1C) as endogenous mRNA levels of RARRES1 transcript variants, measured by RT-PCR according to Example 1-2 using isoform-specific primers for RARRES1.

FIGS. 2A, 2B, and 2C illustrate the silencing of RARRES1 mediated by hypermethylation in a prostate cancer cell line (2A), a lung cancer cell line (2B), and a breast cancer cell line (2C).

FIG. 3A illustrates results of analyzing the effects of RARRES1 transcript variants on cell growth in MDA-MB-231 cells transiently transfected using a method of Example 1-1.

FIG. 3B illustrates results of analyzing the effects of RARRES1 transcript variants on cell growth in JIMT-1 cells transfected with indicated siRNA according to Example 1-4, wherein the transfection was performed using the method of Example 1-1.

FIG. 4A illustrates results of analyzing DNA contents of MDA-MB-231 cells stained with PI 24 hours after being transfected using the method of Example 1-1, with vectors expressing of RARRES1 transcript variants, by flow cytometry according to Example 1-10, wherein an empty vector pcDNA3.1 was used as a control.

FIG. 4B illustrates cell cycle distributions determined by FACS analysis in HEK293 cells when each or all of RARRES1 isoforms were overexpressed.

FIG. 5A illustrates live cell images according to Example 1-6 of GFP-empty vector pcDNA3.1 (Ctrl) or GFP-RARRES1 overexpressed in the progression of 293 cells through the cell cycle.

FIG. 5B illustrates an experimental set-up for monitoring fluorescence-labeled cells by time-lapse microscopy up to 72 hours.

FIG. 5C illustrates results of tracking the fluorescence of individual 293 cells and analyzing the fluorescence by time-lapse fluorescence microscopy for 0 hours, 53 hours, and 72 hours.

FIG. 5D illustrates results of analyzing cell cycle distributions of HEK293 cells by flow cytometry according to Example 1-10, at indicated time points (time) after release from DTB according to Example 1-7.

FIG. 5E illustrates results of measuring overexpression levels by RT-PCR and western blotting, after HEK293 cells were synchronized at the G1/S boundary using a DTB method.

FIG. 6A illustrates results of obtaining cell lysates from pcDNA3.1 (Ctrl)-transfected 293 cells or 293 cells transfected with RARRES1 using the method of Example 1-1 at the indicated time points after DTB release according to Example 1-7, and blotting these cell lysates with the indicated antibodies.

FIG. 6B illustrates results of measuring Cyclin B1 mRNA and protein levels by RT-PCR according to Example 1-2 and immunoblotting (IB) according to Example 1-8.

FIG. 7A illustrates western blotting analysis results for GST, RARRES1, and CDK1 of lysates (input) and GST-IP in 293 cells co-transfected with RARRES1 and GST-Cyclin B1 using the method of Example 1-1.

FIG. 7B illustrates results of analyzing the expression of GST, RARRES1, and Cyclin B1, after HEK293 cells transfected with RARRES1, and GST-CDK1 according to the method of Example 1-1, and cell lysates were immunoprecipitated (IP) with GST beads, according to Example 1-8.

FIG. 7C illustrates results of immunoblotting using the indicated antibodies, after 293 cells transfected with RARRES1 using the method of Example 1-1 were treated with nocodazole (50 ng/ml) or left untreated, and cell lysates were immunoprecipitated with an anti RARRES1 antibody, according to Example 1-8.

FIGS. 8A and 8B illustrate results of probing Cyclin D, CDK4, and RARRES1 (FIG. 8A) or Cyclin A, CDK2, and RARRES1 (FIG. 8B) with a specific antibody, after proteins from HEK293 cells transfected, using the method of Example 1-1, with a plasmid according to Example 1-3 encoding RARRES1 treated with or not treated with 50 ng/ml of nocodazole for 20 hours were immunoprecipitated (IP) with an anti-RARRES1 antibody, according to Example 1-8.

FIG. 9A illustrates mutants obtained by sequentially deleting 50 proteins from the N-terminal of the RARRES1 protein (isoform 1).

FIG. 9B illustrates results of immunoblotting using antibodies against RARRES1 and CDK1, after HEK293 cells transfected with CDK1 to which RARRES1 and His of the mutants prepared in FIG. 9A were bound were precipitated with nickel.

FIG. 10 illustrates results of identifying an amount of the CDK1 protein by RARRES1 when cells were treated with BafA1 and E/P, which are lysosome degradation inhibitors, and MG132, which is a proteasome degradation inhibitor.

FIG. 11 is a view for explaining the fact that the RARRES1 protein causes instability of the CDK1 or Cyclin B1 protein through binding thereto during the cell cycle and also suppresses carcinogenesis through inhibition of the activity thereof.

FIG. 12A illustrates a strategy for producing a targeted Rarres1 allele.

FIG. 12B is a graph showing results of confirming a Rarres1 gene defect by extracting RNA from Rarres1 knockout embryos.

FIG. 12C is a graph showing results of confirming a Rarres1 exon 3 deletion by extracting DNA from Rarres1 knockout embryos.

FIG. 12D is an image showing results of confirming the genotypes of wild-type and Rarres1-deficient mice through PCR genotyping of the tail genomic DNA of mice according to a method of Example 8-2.

FIG. 12E is an image showing results of confirming Rarres1 gene expression in different genotypes through RT-PCR according to a method of Example 1-2 using cDNA prepared from RNA of Rarres1^{+/+}, Rarres1^{+/-}, and Rarres1^{-/-} mouse embryo fibroblasts (MEFs), exon 1 (sense) and exon 6 (antisense), and specific oligonucleotides.

FIG. 12F is an image showing results of analyzing RARRES1 expression in whole embryos at embryonic day 13.5 (E13.5) through western blotting.

FIG. 12G illustrates LacZ staining results for heterozygous embryos from embryonic day 11.5 (E11.5) to embryonic day 14.5 (E14.5) according to Example 8-4.

FIG. 13 illustrates results of analyzing the phenotypes of leucocytes isolated from the bone marrow, spleen, and peripheral blood of wild-type and Rarres1^{-/-} mice at the same age of 18 months.

FIG. 14A illustrates a Kaplan-Meier cancer-free survival curve of age-matched wild-type, Rarres1^{+/-}, and Rarres1^{-/-} mice.

FIG. 14B illustrates gross morphology and histopathology for spontaneous tumors in Rarres1 heterozygous and homozygous mice, through hematoxylin and eosin (H&E) staining.

FIG. 14C illustrates the histopathology of spontaneous tumors in the stomach, liver, lungs, and thyroid of Rarres1^{-/-} mice.

FIG. 14D illustrates the result of immunohistochemistry for T cell-specific marker, CD3, in systemic lymphoma relate to various organs of Rarres1^{-/-} mice.

FIG. 14E illustrates the result of immunohistochemistry for a specific marker myeloperoxidase (MPO) to confirm the proportion of myeloid-series cells in the bone marrow and spleen of wild-type mice and Rarres1^{-/-} mice.

FIG. 15 illustrates results of confirming the shapes and relative weights of the spleen, liver, and kidneys of Rarres1^{+/+}, Rarres1^{+/-}, and Rarres1^{-/-} mice aged between 18 months and 19 months.

FIG. 16 illustrates results of monitoring the amounts of glucose uptake in wild-type (WT) and knockout (KO) mice through [¹⁸F] FDG PET/CT imaging according to Examples 8-5.

FIG. 17 illustrates immunoblotting results for cell lysates derived from wild-type MEFs or Rarres1 deficient MEFs by using the indicated antibodies, according to Example 1-8.

FIG. 18A is a growth curve showing the genotype of each case, obtained by seeding MEF cells (2×10⁴ cells/each genotype) and counting the MEF cells at the indicated time.

FIG. 18B illustrates flow cytometry analysis results for WT and KO MEF cells synchronized at G0 by serum starvation (0.1% FBS) and released in fresh medium (10% FBS) for the indicated time, according to Example 1-10.

FIG. 18C illustrates western blotting analysis results for WT and KO MEF cells with the indicated antibodies and in the same manner as described in FIG. 18B.

FIG. 18D illustrates flow cytometry analysis results for Rarres1^{+/+} and Rarres1^{-/-} MEFs synchronized at prometaphase by nocodazole (80 ng/ml, 12 hour) and released in normal medium containing 10% FBS for the indicated time, according to Example 1-10.

FIG. 18E illustrates results of confirming the expression and phosphorylation of the CDK1 and Cyclin B1 proteins in Rarres1^{+/+} and Rarres1^{-/-} MEF cells treated in the same manner as described in FIG. 18D.

FIG. 19A illustrates results of confirming the occurrence of mitotic errors in 1^{-/-} MEFs.

FIG. 19B illustrates results of staining fibroblasts of wild-type (WT) or Rarres1^{-/-} embryos according to Example 8-3 with antibodies against α-tubulin (red), phalloidin (green), and DAPI (blue) at in vitro embryonic day 13.5 (E13.5).

FIG. 19C illustrates representative images showing mitotic errors in WT and KO fibroblasts according to Example 8-3, and results obtained by quantifying them.

FIG. 19D illustrates results obtained by quantifying the proportion of gamma H2AX-stained micronuclei cells in whole micronuclei cells, in the WT and KO MEFs counter-stained with the antibody against gamma H2AX (red) and DAPI (blue) in FIG. 19A.

FIG. 20A illustrates flow cytometry analysis results for WT and KO MEFs treated with 50 or 100 ng/ml of nocodazole or DMSO for 48 hours, according to Example 1-10.

FIG. 20B illustrates results of quantifying the amounts of sub-G1 DNA from PI-stained cells treated in FIG. 20A.

FIG. 20C illustrates results of measuring a total number of the WT and KO MEFs treated in FIG. 20A.

FIG. 20D illustrates results of western blotting analysis for the PARP and Caspase 3 proteins in MEFs treated with nocodazole (50 ng/ml or 100 ng/ml) for 40 hours.

FIG. 21A illustrates histopathological analysis results for liver and lung sections obtained from WT and Rarres1 KO mice stained with phospho-Cyclin B1-ser126 or phospho-CDK1-T161, wherein the mice were normal mice or tumor-bearing mice.

FIG. 21B illustrates the result of immunohistochemistry for a proliferation marker, ki67, CDK1, and a phosphorylated retinoblastoma (Rb) protein of serially sectioned slides of the lungs of wild-type mice, Rarres1^{+/-} mice, and Rarres1^{-/-} mice, wherein all of the mice had the same age (18 months old).

FIG. 22A illustrates the result of immunohistochemistry for type 2 alveolar cell marker (surfactant protein C; SP-C) and proliferation marker (Ki67), which presents a comparison in proliferative activity of type 2 alveolar cells between the lungs of wild-type mice and Rarres1^{-/-} mice that had the same age (18 months old).

FIG. 22B illustrates the result of immunohistochemistry for Mist1 and LGR5, which are known as organic-specific stem cell markers, of the stomach of wild-type mice and stomach-specific Rarres1 deficient mice.

FIG. 22C illustrates the result of immunohistochemistry for Mist1 and CDK1 of the stomach of wild-type mice, Rarres1^{+/-} mice, and Rarres1^{-/-} mice, wherein all of the mice had the same age (18 months old).

FIG. 22D illustrates the result of spheroid formation assay for confirming the effect of CDK1 inhibition by RO3306 on stemness of embryonic epithelial cells from wild-type and Rarres1^{-/-} mice.

FIG. 22E illustrates quantification results of spheroid formation assay in FIG. 22D.

FIG. 22F illustrates the effect of Rarres1 on stemness in organoid culture using gastric epithelial cells isolated from the stomach of wild-type mice and Rarres1^{-/-} mice.

FIG. 22G illustrates prepared lungs for RNQ sequencing of wild-type mice and Rarres1^{-/-} mice at in vitro embryonic day 19 (E19), at 10 months old, and at 18 months old.

FIG. 23 illustrates results of analyzing a copy number variant (CNV) for each tumor sample.

FIG. 24A illustrates differentially expressed genes and upregulated genes, involved in WNT signaling and mitosis pathways.

FIG. 24B illustrates pathway activity estimated from results confirming that when WT was compared with KO in terms of an unfolded protein response (UPR), WT exhibited activity opposite to that activated in a KO tumor.

FIG. 24C illustrates the mRNA expression state of Hspa8 in the UPR and high binding affinity confirmed as a result of an IgG experiment.

FIG. 24D illustrates results of gene cluster enrichment analysis using differentially expressed genes.

FIG. 25A illustrates CDK1 mRNA, CDK1 protein expression, and the correlation therebetween, to evaluate the characteristics of genomes of lung adenocarcinoma expressed in people.

FIG. 25B illustrates Rarres1mRNA expression for each isoform.

FIG. 25C illustrates low Rarres1 group C1 exhibiting downregulation in the cell cycle pathway.

FIG. 25D illustrates mouse data sets and estimation of the presence of lung cells using human lung adenocarcinoma.

FIG. 25E illustrates results of analyzing gene expression characteristics of 6 subtypes of human lung cancer (TCGA LUAD).

FIG. 26 schematically illustrates a cancer-inducing mechanism in Rarres1 deficient mice, which indicates that when there is no Rarres1, abnormal activation of CDK1, which is a mitosis phase regulatory protein, causes overall abnormalities in the cell cycle, resulting in the occurrence of cancer.

### BEST MODE FOR CARRYING OUT THE INVENTION

As a result of having conducted intensive studies to discover molecular mechanisms for diagnosing cancer and predicting a prognosis, the inventors of the present invention confirmed that in cancer-derived samples, according to the degree of mutual binding between retinoic acid receptor responder 1 (RARRES1) and Cyclin-dependent kinase 1 (CDK1) or Cyclin B1, the mitosis of cancer cells was arrested, the formation of CDK1-Cyclin B1 complexes was suppressed, and the degradation of these proteins was promoted, and thus they were crucial factors in the diagnosis of cancer, prognosis prediction, and the treatment of cancer, and thus completed the present invention based on these findings.

Hereinafter, the present invention will be described in detail.

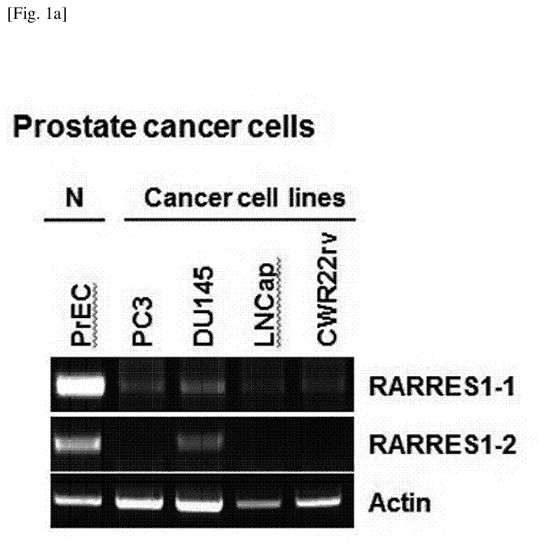

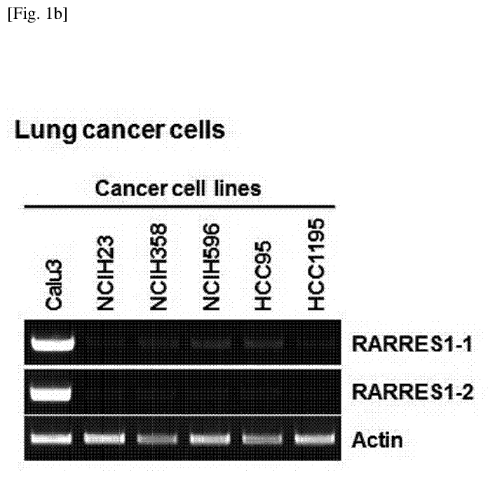

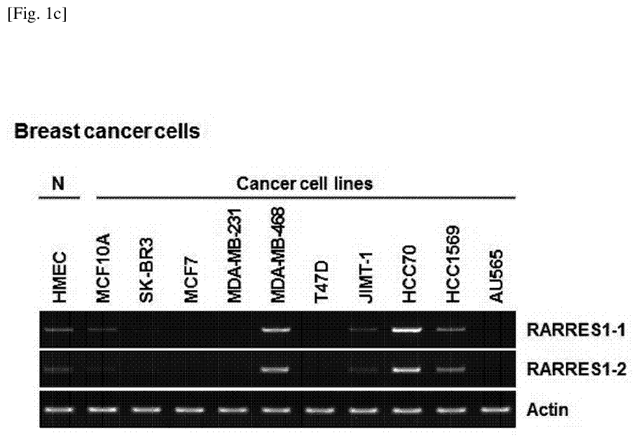

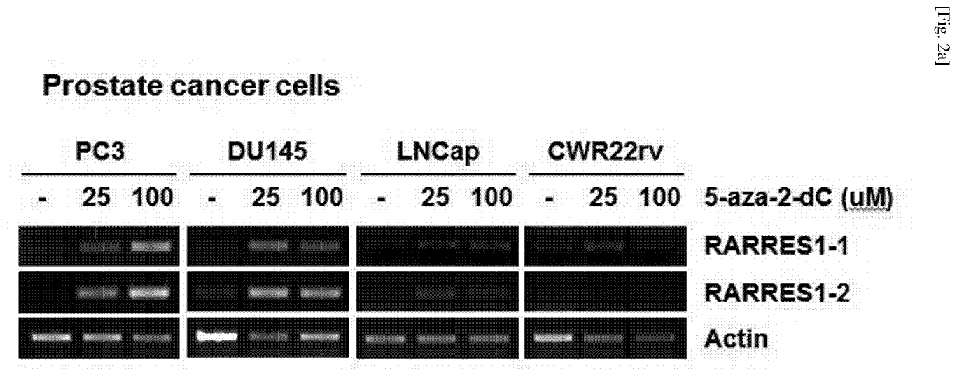

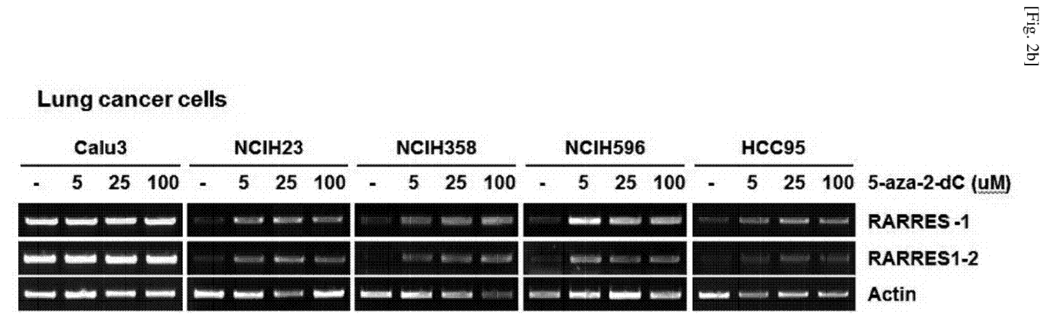

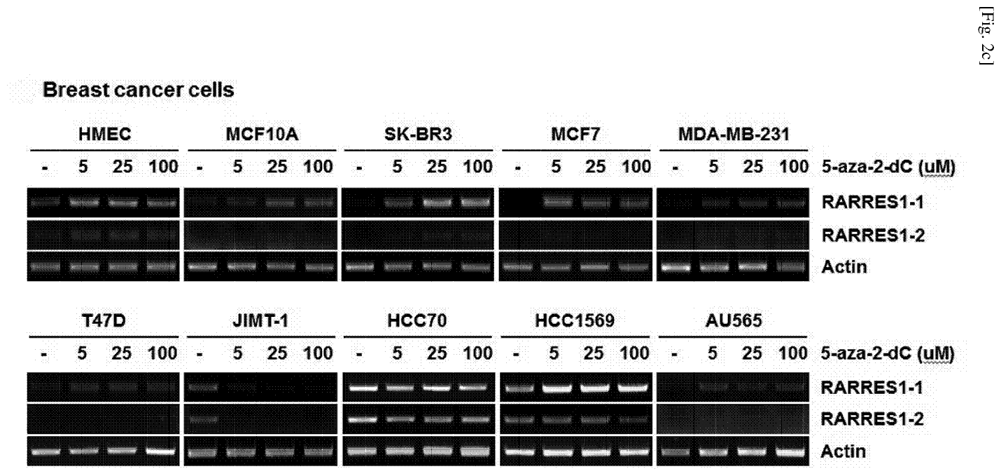

In one embodiment of the present invention, as a result of analyzing expression levels of RARRES1 isoforms in prostate cancer, lung cancer, and breast cancer cell lines in order to examine whether RARRES1 was non-activated in human cancer cell lines, it was confirmed that the expression of RARRES1 was silenced (see FIGS. 1A to 1C), and as a result of treating 5-aza-2′-deoxycytidine (5-aza-2-dC; 5, 25, and 100 uM) with an inhibitor of DNA methylation for 5 days in order to determine whether promoter methylation was associated with silencing of the expression of RARRES1 in cancer cells, it was confirmed that although the expression levels of RARRES1 were different from one other in most cell lines, the expression of RARRES1 was recovered in a dose-dependent manner (see FIGS. 2A to 2C). From these results, it was confirmed that the inhibition of RARRES1 in prostate cancer, lung cancer, and breast cancer cell lines was mediated by methylation of the RARRES1 gene (see Example 2).

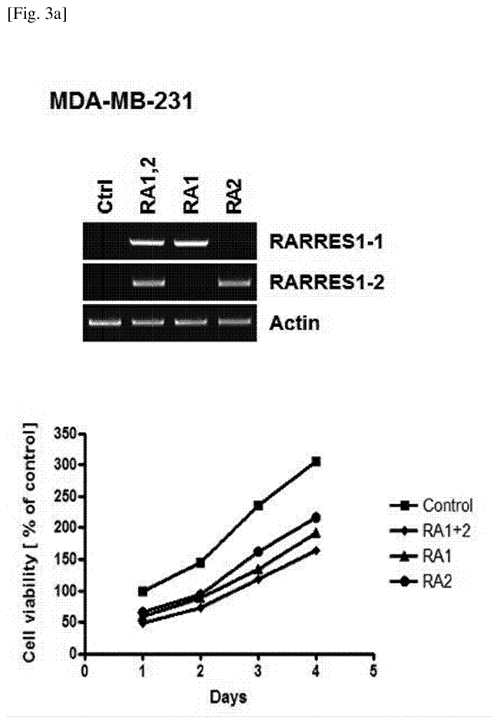

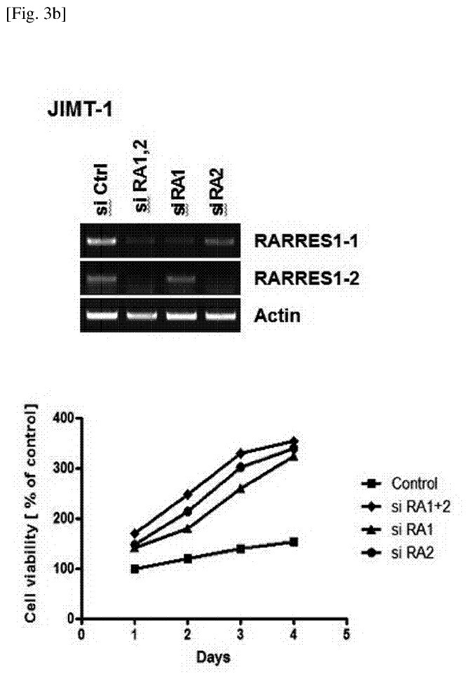

In another embodiment of the present invention, as a result of examining the effect of RARRES1 on cell proliferation according to Example 1-5 in breast cell lines MDAMB-231 and JIMT-1 in order to determine whether RARRES1 acts as a tumor suppressor gene, it was confirmed that although cancer cell growth was gradually inhibited for a certain period of time after transfection with both or each of RARRES1 isoform expression vectors, according to a method of Example 1-1 (see FIG. 3A), RARRES1 mRNA expression was reduced by transfection with a specific siRNA according to Example 1-4 for RARRES1 variants, by using the method of Example 1-1, in JIMT-1 cells, and cancer cell viability was enhanced in RARRES1-depleted cells, which indicates that RARRES1 acts as a tumor suppressor gene (see Example 3).

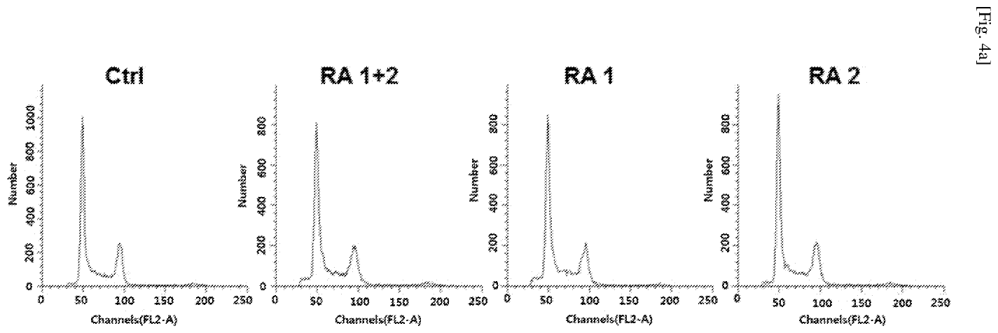

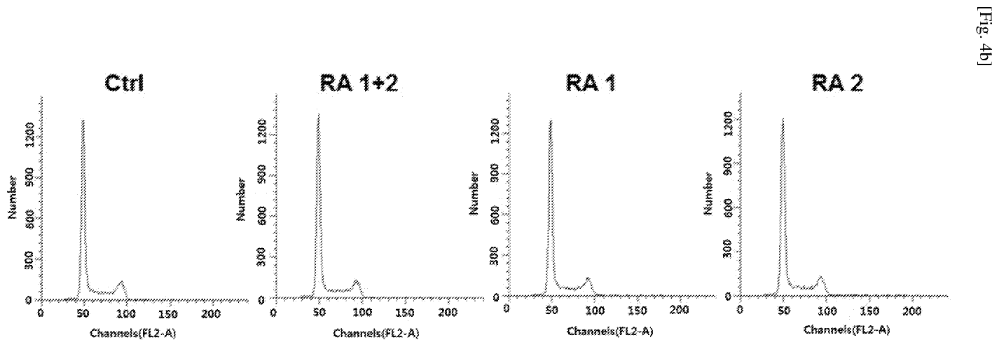

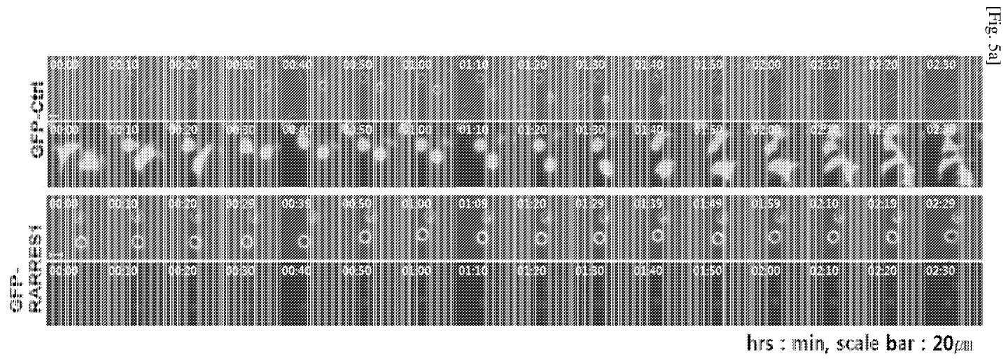

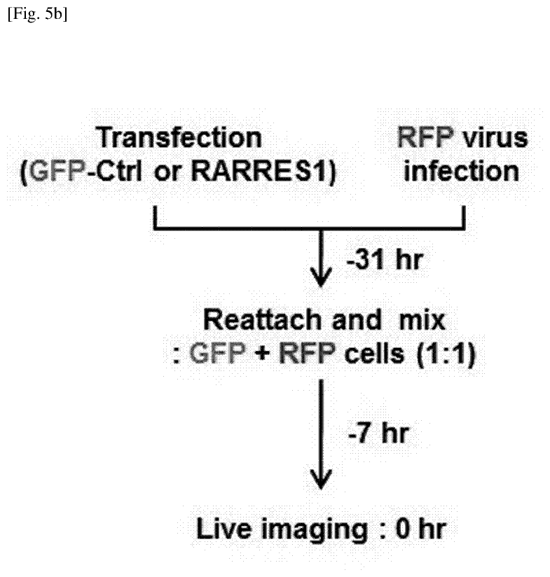

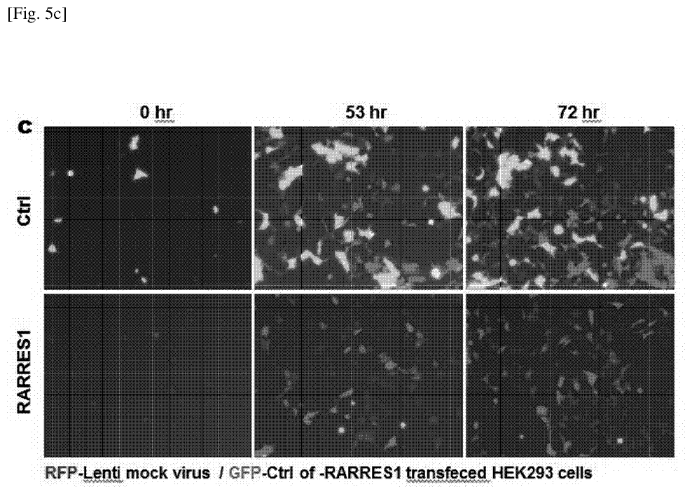

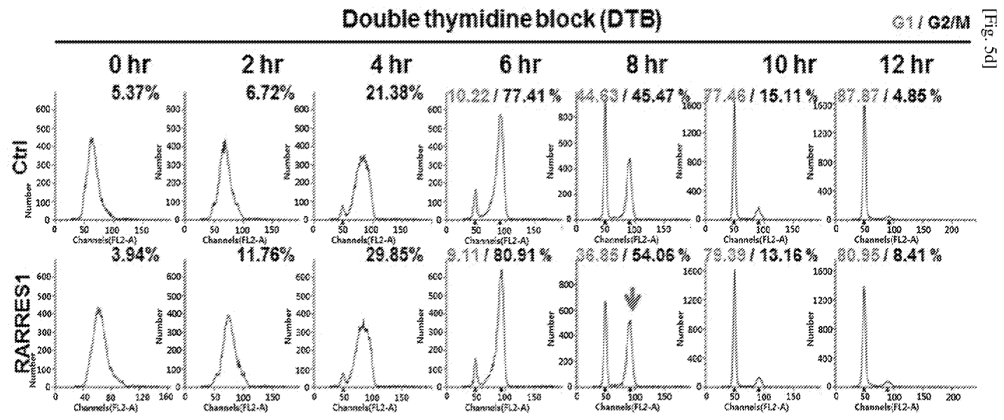

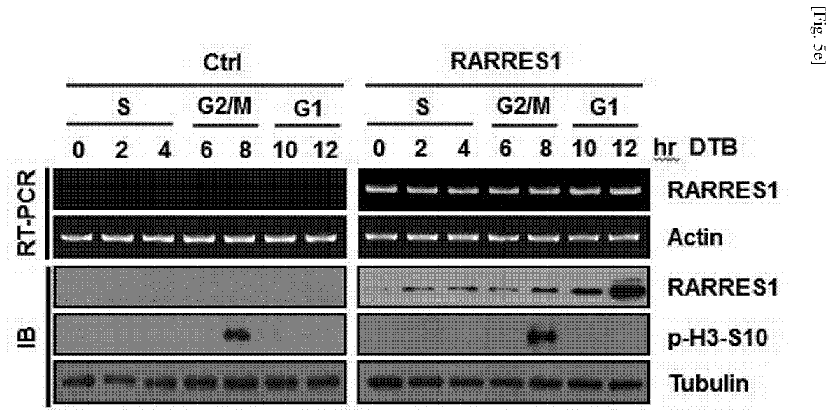

In another embodiment of the present invention, as a result of overexpressing RARRES1 in MDA-BM-231 and HEK293 cancer cells, and then performing flow cytometry according to Example 1-10 thereon in order to confirm that mitotic arrest of cancer cells was induced by overexpression of RARRES1, apoptosis was not induced (see FIGS. 4A and 4B). On the other hand, as a result of transfecting HEK293 cells with a green fluorescence protein (GFP) tagged with RARRES1 or an empty vector (Ctrl), by using the method of Example 1-1 and observing the cells by live cell imaging according to Example 1-6, it was confirmed that mitotic arrest was induced when RARRES1 was overexpressed in the HEK293 cells (see FIGS. 5A to 5E), and as a result of performing western blotting analysis using a double thymidine block (DTB) method according to Example 1-7 in order to examine which a cell cycle regulatory protein was modified in RARRES1-overexpressing cells, it was confirmed that the Rb protein and Rb phosphorylation (ser 807,811), the expression and phosphorylation of Cyclin B1 (serine 126), which indicates that the overexpression of RARRES1 is associated with the activity of Cyclin B1 (see Example 4).

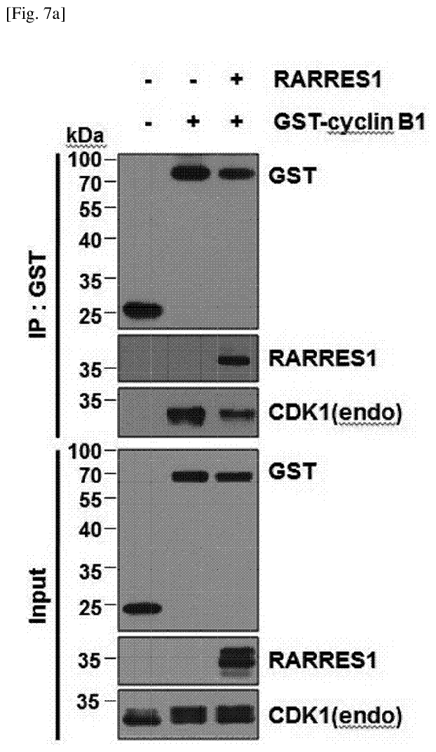

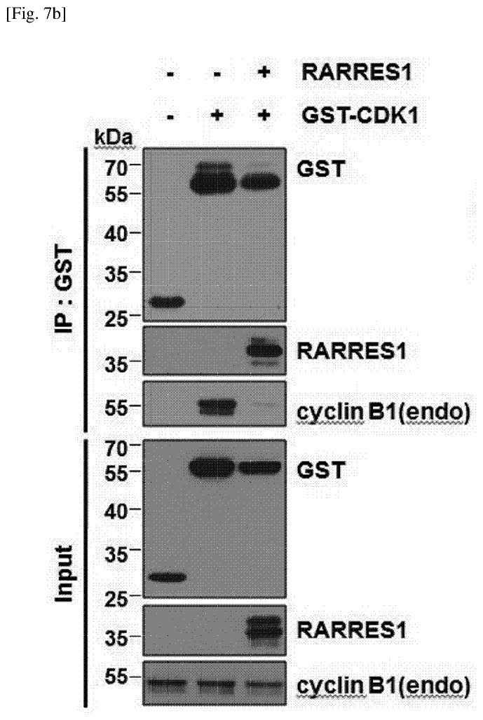

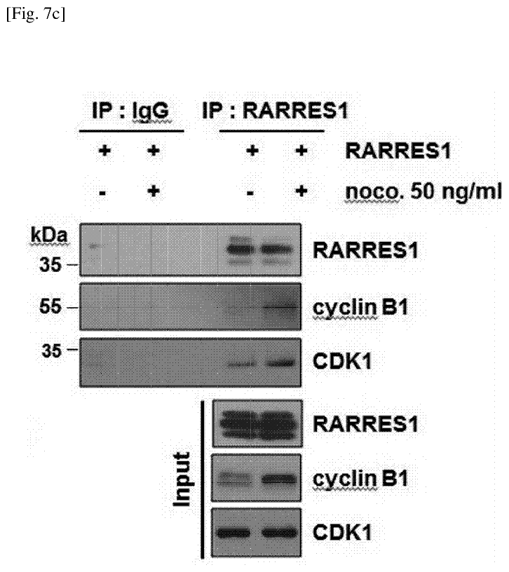

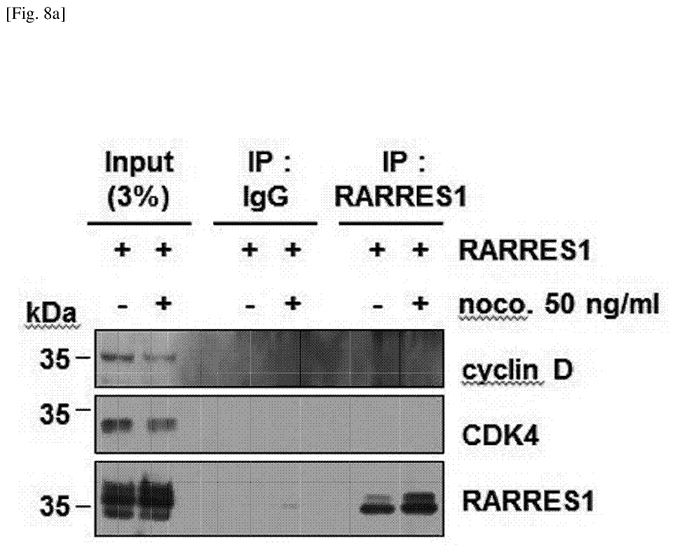

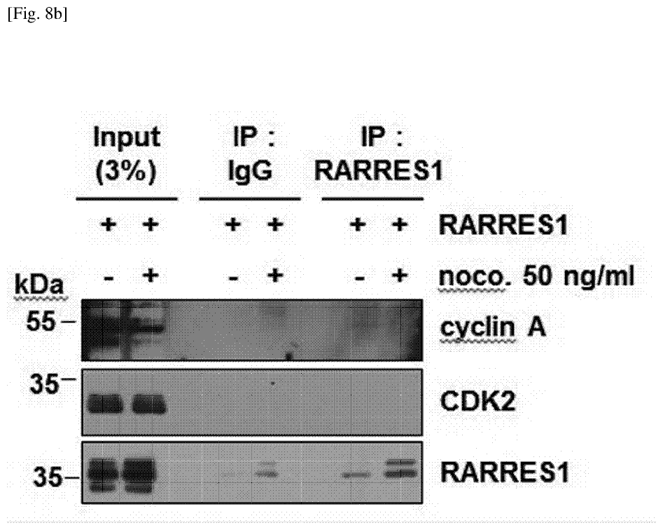

In another embodiment of the present invention, as a result of co-transfecting HEK293 cells with RARRES1 and Cyclin B1 by using the method of Example 1-1 and immunoprecipitating cell lysates with GST beads, according to Example 1-8 to test whether RARRES1 influenced Cyclin B1 activation through direct binding during mitosis, it was confirmed that RARRES1 directly bound to Cyclin B1 (see FIG. 7A), and it was also confirmed that RARRES1 directly interacted with CDK1 (see FIG. 7B), and as a result of performing immunoprecipitation according to Example 1-8 on 293 cells to examine whether RARRES1 interacts with other CDK-Cyclin complexes, including interphase CDKs (CDK2, CDK4, and CDK6) and Cyclins binding thereto, it was confirmed that, while each component of CDK4-Cyclin D and CDK2-Cyclin A complexes rarely bind to RARRES1 (see FIGS. 8A and 8B), RARRES1 specifically inhibited the formation of a CDK1-Cyclin B1 complex in mitosis (see Example 5).

In another embodiment of the present invention, as a result of preparing RARRES1 protein mutants having sequences with different sequences of 50 deletions and performing immunoprecipitation thereon to search for an amino acid region of the RARRES1 protein that binds to CDK1, it was confirmed that binding to CDK1 hardly occurred in mutants with the deletion of amino acids 251 to 294 at the C-terminus of RARRES1, and thus the C-terminus of RARRES1 acted as a crucial site for CDK1 binding (see Example 6).

In another embodiment of the present invention, as a result of treating and observing a lysosomal degradation inhibitor and a proteasome degradation inhibitor to examine how a quantitative change in the CDK1 protein is regulated, it was confirmed that an amount of the CDK1 protein was increased again upon treatment with the lysosomal degradation inhibitor, and thus the binding of RARRES1 to CDK1 through the C-terminus thereof caused instability of the CDK1 protein through lysosomes (see Example 7).

Thus, it was confirmed that the formation of a CDK1-Cyclin B1 complex was inhibited according to the degree of mutual binding between RARRES1 and CDK1 or Cyclin B1, thereby causing mitotic arrest of cancer cells and suppressing an increase in cancer cells.

Therefore, the present invention provides a method of screening for a cancer therapeutic agent, including: (a) treating a sample with candidate materials in vitro; (b) measuring a degree of binding between CDK1 and Cyclin B1 of the sample or measuring an amount or activity of the CDK1 protein or the Cyclin B1 protein; and (c) selecting, as a cancer therapeutic agent, a candidate material exhibiting a decrease in the degree of binding between CDK1 and Cyclin B1, or a candidate material exhibiting a decrease in the amount or activity of the CDK1 protein or the Cyclin B1 protein, as compared to that in a group not treated with the candidate materials.

The method may further include, in the process (b), measuring a degree of binding between retinoic acid receptor responder 1 (RARRES1) and CDK1 or Cyclin B1 of the sample; and, in the process (c), selecting, as a cancer therapeutic agent, a candidate material exhibiting a decrease in the degree of binding between CDK1 and Cyclin B1 and an increase in the degree of binding between RARRES1 and CDK1 or Cyclin B1, but the present invention is not limited thereto. Ultimately, in the process (c), it is characterized that RARRES1 inhibits the formation of the CDK1-Cyclin B1 complex by binding to CDK1 or Cyclin B1, but the present invention is not limited thereto.

In addition, in the process (c), a candidate material exhibiting a decrease in the amount or activity of the CDK1 protein or the Cyclin B1 protein may be selected as a cancer therapeutic agent, and preferably, the decrease in the amount or activity of the CDK1 protein may be an increase in the degradation of CDK1 in lysosomes due to an increased degree of binding between RARRES1 and CDK1, and thus RARRES1 may affect stability by inducing the degradation of the CDK1 protein, but the present invention is not limited thereto.

In addition, the process (b) may be performed by a polymerase chain reaction (PCR), a microarray, northern blotting, western blotting, an enzyme-linked immunosorbent assay (ELISA), immunoprecipitation, immunohistochemistry, or immunofluorescence, but the present invention is not limited thereto.

In addition, the sample may be derived from one or more selected from the group consisting of prostate cancer, lung cancer, and breast cancer patients, but the present invention is not limited thereto, and the sample may be one or more selected from the group consisting of tissue, cells, whole blood, blood, saliva, sputum, cerebrospinal fluid, and urine, but the present invention is not limited thereto.

In addition, the decrease in the degree of binding between CDK1 and Cyclin B1 is characterized by inhibition of the phosphorylation of serine 126 of the Cyclin B1 protein, but the present invention is not limited thereto, and the increase in the degree of binding between RARRES1 and CDK1 may be due to bindability of RARRES1 to inactivated CDK1 at the C-terminal containing amino acids 251 to 294 of the RARRES1 protein, and the increase in the degree of binding between RARRES1 and Cyclin B1 may be due to bindability of RARRES1 to Cyclin B1 regardless of the RARRES1 protein isoform. Preferably, an amino acid region of RARRES1 that binds to Cyclin B1 may be any region of the RARRES1 protein which is capable of binding to Cyclin B1.

In addition, the Cyclin B1 protein may have an amino acid sequence of SEQ ID NO: 1, and the amino acids 251 to 294 of the RARRES1 protein may have an amino acid sequence of SEQ ID NO: 6, but the present invention is not limited thereto.

In the present invention, in addition to the amino acid sequence of SEQ ID NO: 1, any amino acid sequence corresponding to amino acids with a reduced degree of binding to CDK1 as amino acids of the Cyclin B1 protein may be used.

In the present invention, in addition to the amino acid sequence of SEQ ID NO: 6, any amino acid sequence corresponding to amino acids that bind to inactivated CDK1 as amino acids belonging to the RARRES1 protein may be used.

In addition, the candidate materials refer to unknown materials used in screening in order to measure the degree of binding between CDK1 and Cyclin B1, the degree of binding between RARRES1 and CDK1 or Cyclin B1, and the amount or activity of the CDK1 protein or the Cyclin B1 protein, and preferably may be one or more selected from the group consisting of compounds, microorganism cultures or extracts, natural extracts, nucleic acids, and peptides, but the present invention is not limited thereto. The nucleic acids may be one or more selected from the group consisting of aptamers, locked nucleic acids (LNAs), peptide nucleic acids (PNAs), and morpholinos, but the present invention is not limited thereto.

In addition, the measurement of the degree of binding between CDK1 and Cyclin B1 of the sample, the degree of binding between RARRES1 and CDK1 or Cyclin B1, and the amount or activity of the CDK1 protein or the Cyclin B1 protein may be performed by a polymerase chain reaction (PCR), a microarray, northern blotting, western blotting, an enzyme-linked immunosorbent assay (ELISA), immunoprecipitation, immunohistochemistry, or immunofluorescence, but the present invention is not limited thereto.

According to another embodiment of the present invention, there are provided a composition for diagnosing cancer or predicting a prognosis, which includes an agent for measuring an mRNA level of RARRES1 or a level of a peptide encoded by the RARRES1 gene, and a kit for diagnosing cancer or predicting a prognosis, which includes an agent for measuring an mRNA level of RARRES1 or a level of a peptide encoded by the RARRES1 gene.

The mRNA of the RARRES1 gene of the present invention may have a base sequence of SEQ ID NO: 4 or 5, and may include a nucleotide of a base sequence of SEQ ID NO: 7, but the present invention is not limited thereto, and the peptide encoded by the RARRES1 gene may have an amino acid sequence of SEQ ID NO: 2 or 3, and may include a peptide having an amino acid sequence of SEQ ID NO: 6, but the present invention is not limited thereto.

The term “diagnosis” as used herein means, in a broad sense, determining conditions of disease of a patient in all aspects. Content of the determination includes disease name, the cause of a disease, the type of disease, the severity of disease, detailed aspects of syndrome, the presence or absence of complications, prognosis, and the like. In the present invention, diagnosis means determining the presence or absence of the onset of prostate cancer, lung cancer, and breast cancer, the level of progression thereof, and the like.

The term “prognosis” as used herein refers to the progression of a disease and prediction of recovery therefrom, and means an outlook or preliminary evaluation. In the present invention, the prognosis means the recurrence of prostate cancer, lung cancer, and breast cancer, overall survival, or disease-free survival, but the present invention is not limited thereto.

The agent for measuring an mRNA level of the RARRES1 gene may be a sense and antisense primer, or probe that complementarily binds to mRNA, but the present invention is not limited thereto.

The term “primer” as used herein refers to a short nucleic acid sequence that acts as a point of initiation for DNA synthesis, and means an oligonucleotide synthesized for use in diagnosis, DNA sequencing, and the like. The primers may be generally synthesized to a length of 15 base pairs to 30 base pairs, but may vary according to the purpose of use, and may be modified using a known method, such as methylation, capping, or the like.

The term “probe” as used herein refers to a nucleic acid having a length of several to hundreds of bases and capable of specifically binding to mRNA, wherein the nucleic acid is prepared through enzymatic chemical separation and purification or synthesis. A probe may be labeled with a radioactive isotope, an enzyme, or the like to identify the presence or absence of mRNA, and may be designed and modified using a known method.

The agent for measuring the level of the protein may be an antibody specifically binding to a protein encoded by a gene, but the present invention is not limited thereto.

The term “antibody” as used herein includes immunoglobulin molecules which are immunologically reactive with a specific antigen, and includes both monoclonal and polyclonal antibodies. In addition, the antibody includes forms produced by genetic engineering, such as chimeric antibodies (e.g., humanized murine antibodies) and heterologous binding antibodies (e.g., bispecific antibodies).

The kit for diagnosing cancer or predicting a prognosis of the present invention consists of one or more types of other components, solutions or devices suitable for analysis methods.

For example, the kit of the present invention may be a kit including genomic DNA derived from a sample to be analyzed, a primer set specific to a marker gene of the present invention, an appropriate amount of DNA polymerase, a dNTP mixture, a PCR buffer, and water, to perform PCR. The PCR buffer may include KCl, Tris-HCl, and MgCl₂. The kit of the present invention may further include, in addition to the above components, components needed for electrophoresis, which may be used to confirm whether a PCR product is amplified.

In addition, the kit of the present invention may be a kit including essential elements needed for performing RT-PCR. An RT-PCR kit may include, in addition to each pair of primers specific to marker genes, test tubes or other suitable containers, reaction buffers, deoxynucleotides (dNTPs), enzymes such as Taq-polymerase and reverse transcriptases, DNase and RNase inhibitors, DEPC-water, sterile water, and the like. In addition, the RT-PCR kit may include a pair of primers specific to a gene used as a quantitative control.

In addition, the kit of the present invention may be a kit including essential elements needed for performing DNA chip analysis. A DNA chip kit may include a substrate to which a gene or cDNA corresponding to a fragment thereof is attached, and the substrate may include a quantitative structural gene or cDNA corresponding to a fragment thereof. In addition, the kit of the present invention may be in the form of a microarray including a substrate on which a marker gene of the present invention is immobilized.

In addition, the kit of the present invention may be a kit including essential elements needed to perform ELISA. An ELISA kit includes an antibody specific to a marker protein, and an agent for measuring a level of the marker protein. The ELISA kit may include a reagent capable of detecting an antibody forming an antigen-antibody complex, e.g., a labeled secondary antibody, chromophores, an enzyme, and a substrate of the enzyme. In addition, the ELISA kit may include an antibody specific to a protein as a quantitative control.

The term “antigen-antibody complex” as used herein refers to a composite of a protein encoded by a gene and an antibody specific thereto. The formation amount of the antigen-antibody complex may be quantitatively measured by the intensity of a signal of the detection label. The detection label may be selected from the group consisting of an enzyme, a fluorescent substance, a ligand, a luminescent substance, microparticles, a redox molecule, and a radioactive isotope, but the present invention is not limited thereto.

According to still another embodiment of the present invention, there is provided a pharmaceutical composition for preventing or treating cancer, which includes, as an active ingredient, an inhibitor of binding between CDK1 and Cyclin B1.

The term “prevention” as used herein means all actions that inhibit or delay the onset of cancer via preemptive administration of the pharmaceutical composition according to the present invention prior to the onset of cancer.

The term “treatment” as used herein means all actions that improve or beneficially change symptoms of cancer via administration of the pharmaceutical composition according to the present invention after the onset of cancer.

Therefore, the pharmaceutical composition may further include a suitable carrier, excipient or diluent that is commonly used to prepare a pharmaceutical composition. In addition, the pharmaceutical composition may be formulated in the form of oral preparations such as powder, granules, tablets, capsules, suspensions, emulsions, syrups, aerosols, and the like, preparations for external application, suppositories, and sterile injection solutions, according to general methods.

Examples of the suitable carrier, excipient, and diluent that may be included in the composition include lactose, dextrose, sucrose, sorbitol, mannitol, xylitol, erythritol, maltitol, starch, acacia gum, alginates, gelatin, calcium phosphate, calcium silicate, cellulose, methyl cellulose, micro-crystalline cellulose, polyvinyl pyrrolidone, water, methyl hydroxy benzoate, propyl hydroxy benzoate, talc, magnesium stearate, mineral oil, and the like. When the composition is formulated, commonly used diluents or excipients such as a filler, an extender, a binder, a wetting agent, a disintegrating agent, a surfactant, and the like are used.

The pharmaceutical composition according to the present invention is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration routes, excretion rate, treatment period, and simultaneously used drugs, and other factors well known in the medical field.

To enhance therapeutic effects, the pharmaceutical composition according to the present invention may be administered simultaneously, separately, or sequentially with a drug used in combination therewith, and may be administered in a single dose or multiple doses. It is important to administer the pharmaceutical composition in the minimum amount that enables achievement of the maximum effects without side effects in consideration of all the above-described factors, and this may be easily determined by those of ordinary skill in the art. In particular, an effective amount of the pharmaceutical composition according to the present invention may vary according to age, gender, condition, and body weight of a patient, the absorption, inactivity, and excretion rate of active ingredients in the body, the type of disease, and simultaneously used drugs.

The pharmaceutical composition of the present invention may be administered to an individual via various routes. All administration methods may be expected, and may be, for example, oral administration, intranasal administration, transbronchial administration, arterial administration, intravenous administration, subcutaneous injection, intramuscular injection, or intraperitoneal injection. A daily dose of the pharmaceutical composition may be administered once or multiple times a day, but the present invention is not limited thereto.

The pharmaceutical composition of the present invention is determined according to various related factors such as a disease to be treated, administration routes, the age, gender, and body weight of a patient, the severity of disease, and the like, and the type of drug, which is an active ingredient.

In addition, the cancer may be one or more selected from the group consisting of prostate cancer, lung cancer, and breast cancer, but the present invention is not limited thereto.

The term “prostate cancer” as used herein refers to an adenocarcinoma occurring in prostate cells. The type of prostate cancer is classified according to the degree of differentiation of tumor tissues, the characteristics of cells, and the like, and the widely used classification method was proposed by a pathologist named Donald Gleason, and prostate cancer is divided into the highest grade 1 to the lowest grade 5 in terms of the degree of differentiation. 95% of the cases of cancer occurring in the prostate gland are adenocarcinomas occurring in the duct-acinar secretory epithelium, and transitional cell carcinoma, and the like account for 5%. In addition, approximately 85% of adenocarcinomas occur in a region called a peripheral zone in the zone classification of McNeil as seen above. A precancerous change in the prostate is called ‘neoplasm in the prostate epithelium,’ and this is found in about a third of patients with prostate cancer. Among them, highly malignant neoplasms with a poor degree of differentiation are found in 80% of invasive prostate cancer, i.e., cancer with the properties of spreading to neighboring tissues, and thus are regarded as progenitor lesions of prostate cancer.

The term “lung cancer” as used herein refers to a malignant tumor occurring in the lungs, and lung cancer may occur in the lung itself or may occur due to metastasis of cancers occurring in other organs to the lungs. The types of primary lung cancers are classified into non-small cell lung cancer and small cell lung cancer based on the size and shape of cancer cells. Non-small cell lung cancer accounts for 80% to 85% of the cases of lung cancer, and it is subdivided into adenocarcinoma, squamous cell carcinoma, large cell carcinoma, and the like. Small cell lung cancer, the remainder, is generally highly malignant, and thus, at the time of detection, has often metastasized to other organs, the opposite lung, and the mediastinum through lymphatic vessels or blood vessels.

The term “breast cancer” as used herein refers to a malignant tumor that spreads out of the breast and thus is life-threatening. Breast cancer is divided into cancer that occurs in parenchymal tissues such as ducts and lobule, and cancer occurring in other epilepsy tissues, according to the site of occurrence thereof, and Ductal and lobular carcinomas are subdivided into invasive breast cancer and noninvasive breast cancer depending on a degree to which cancer cells spread to the surrounding tissues. The incidence rate of breast cancer in males is 1% or less of the cases of breast cancer in females, and most of the cases are invasive ductal carcinomas.

In addition, as a result of having conducted intensive studies to discover molecular mechanisms for diagnosing cancer and predicting a prognosis, the inventors of the present invention confirmed that a Rarres1^{-/-} animal model was prone to spontaneous tumors and exhibited increased phosphorylation of CDK1 and Cyclin B1 and a high activity of a CDK1-Cyclin B1 complex, and thus it was confirmed that the tumor cell cycle progression was unusually rapid. In addition, the inventors confirmed that chromosomes were unstable upon induction of mitotic defects and mitosis, from which it was confirmed that RARRES1 is a crucial factor in diagnosing cancer, predicting a prognosis, and treating cancer, and thus completed the present invention based on these findings.

Hereinafter, the present invention will be described in detail.

In one embodiment of the present invention, conditional RARRES1 knockout (KO) mice were induced to determine the in vivo physiological function of RARRES1 (see Example 9).

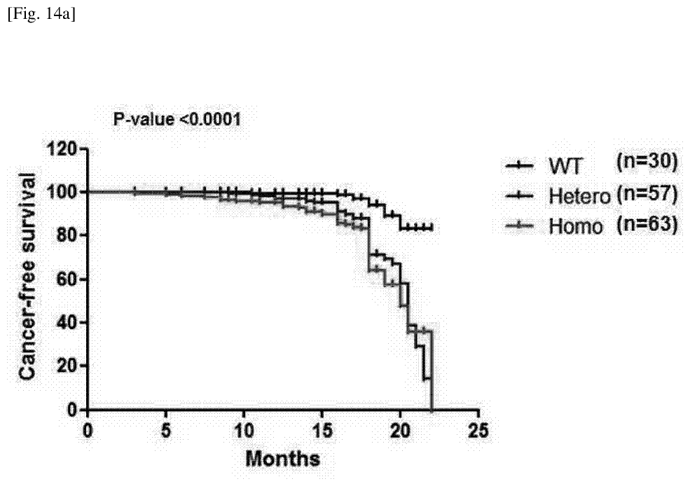

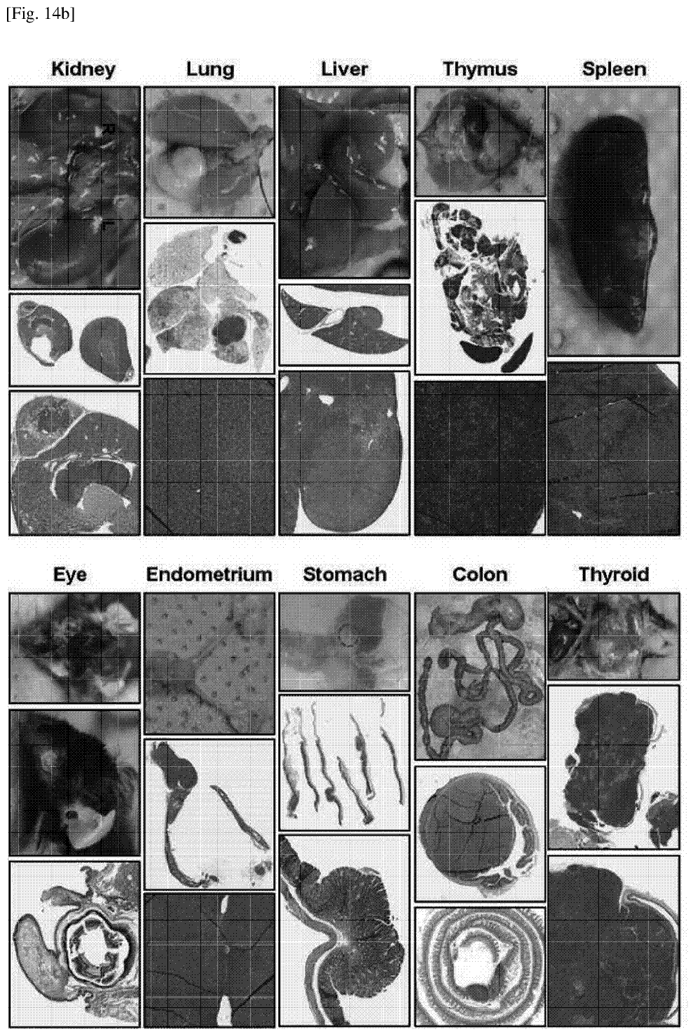

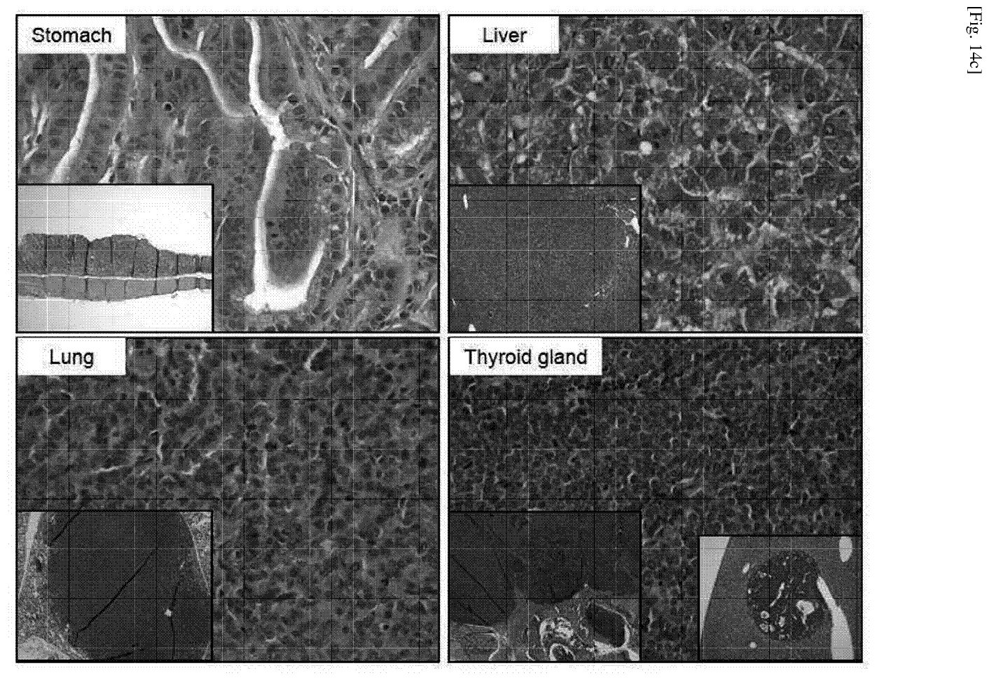

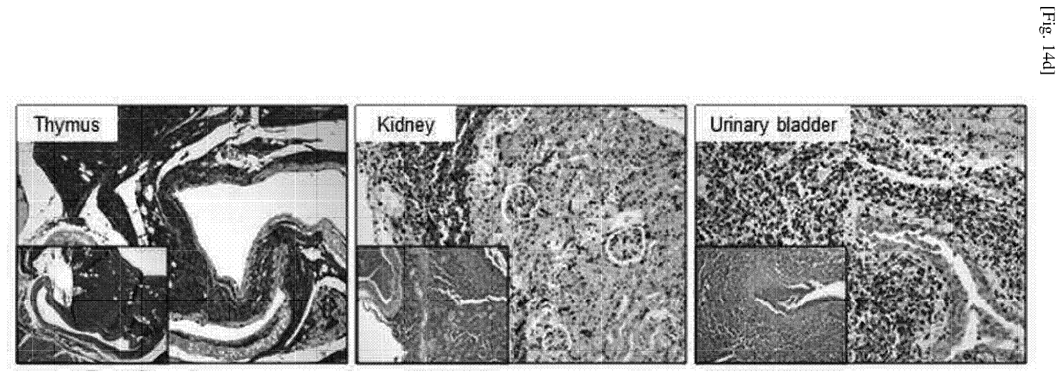

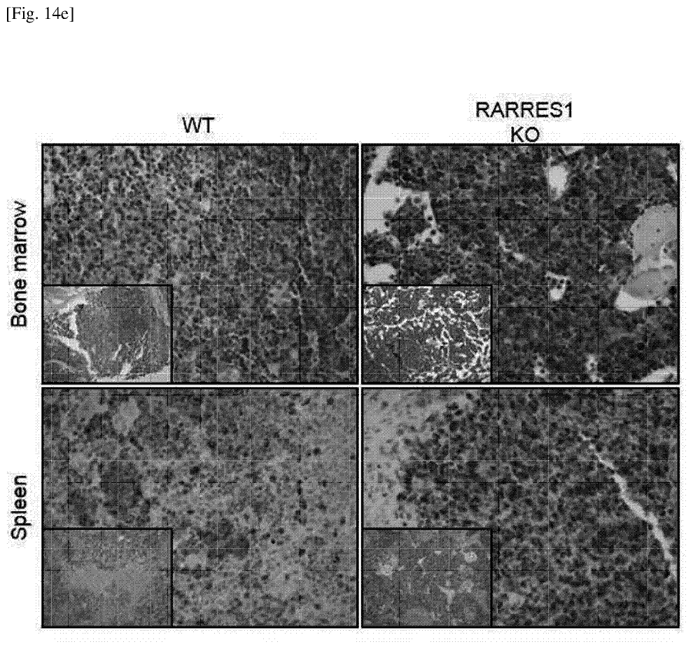

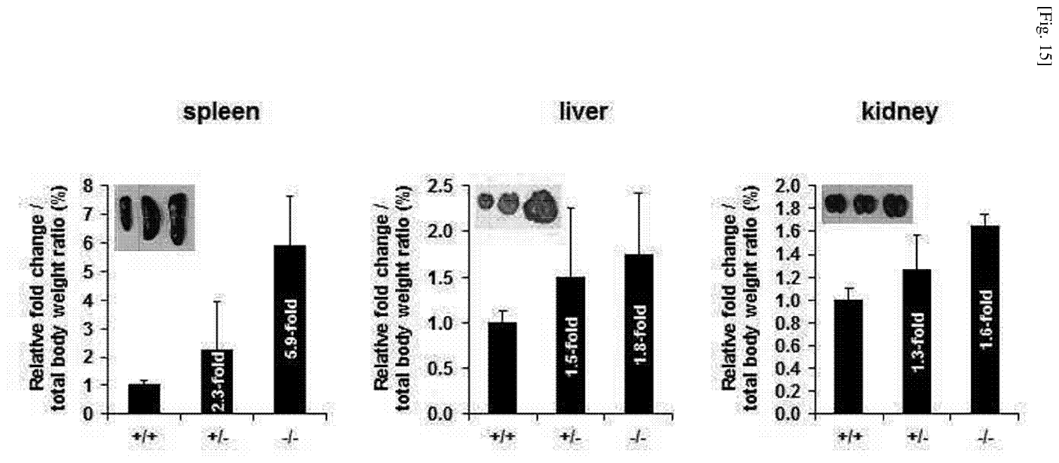

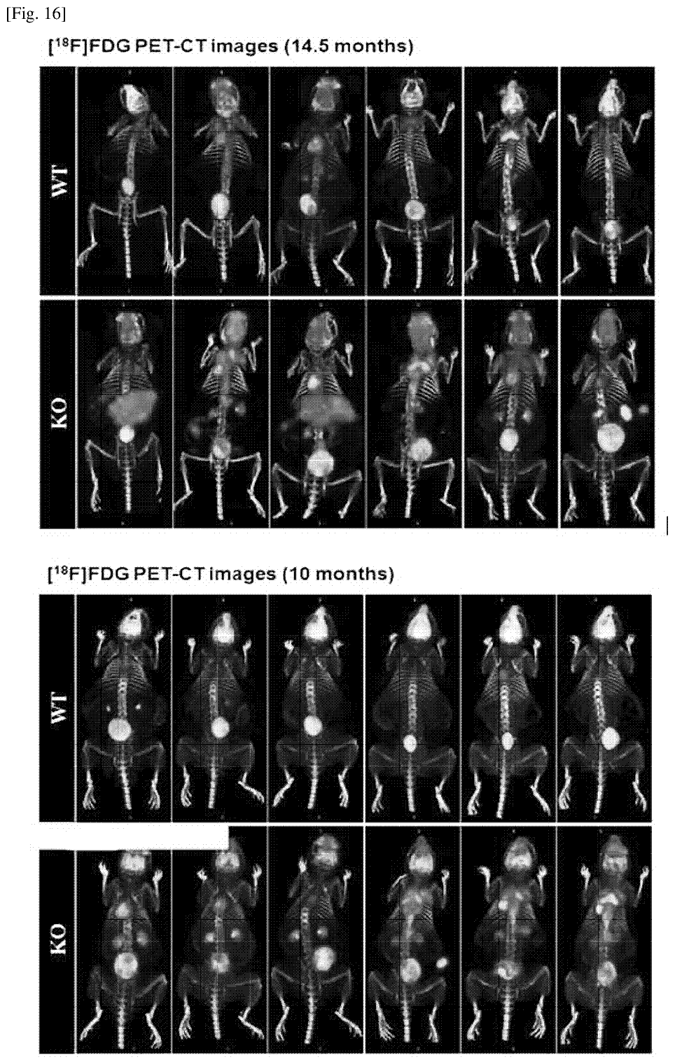

In another embodiment of the present invention, to confirm the spontaneous tumor formation in Rarres1 knockout (KO) mice, as a result of establishing cohorts of Rarres1^{+/+} (n=51), Rarres1^{+/-} (n=47), and Rarres1^{-/-} (n=59) mice for intercrossed Rarres1 heterozygous mice (C57BL/6) and observing the cohorts up to 19 months old, it was found that Rarres1^{+/-} and Rarres1^{-/-} mice were significantly more prone to develop spontaneous tumors than Rarres1^{+/+} mice, and it was confirmed that the Rarres1^{+/-} and Rarres1^{-/-} mice had various types of tumors in organs including the spleen, thymus, liver, lungs, kidneys, thyroid, small intestine, stomach, endometrium, and eyes, which were different from those of the Rarres1^{+/+} mice (see FIG. 14), and the sizes of organs including the spleen, liver, and kidneys were gradually increased in 19-month-old mice according to genotype (see FIG. 15). In addition, it was confirmed through PET/CT imaging according to Example 1-9 that tumors were generated much earlier in Rarres1 KO mice than in wild type (WT) mice (see FIG. 16), and it was confirmed that tumorigenesis was induced only by deletion of the Rarres1 gene (see Example 11).

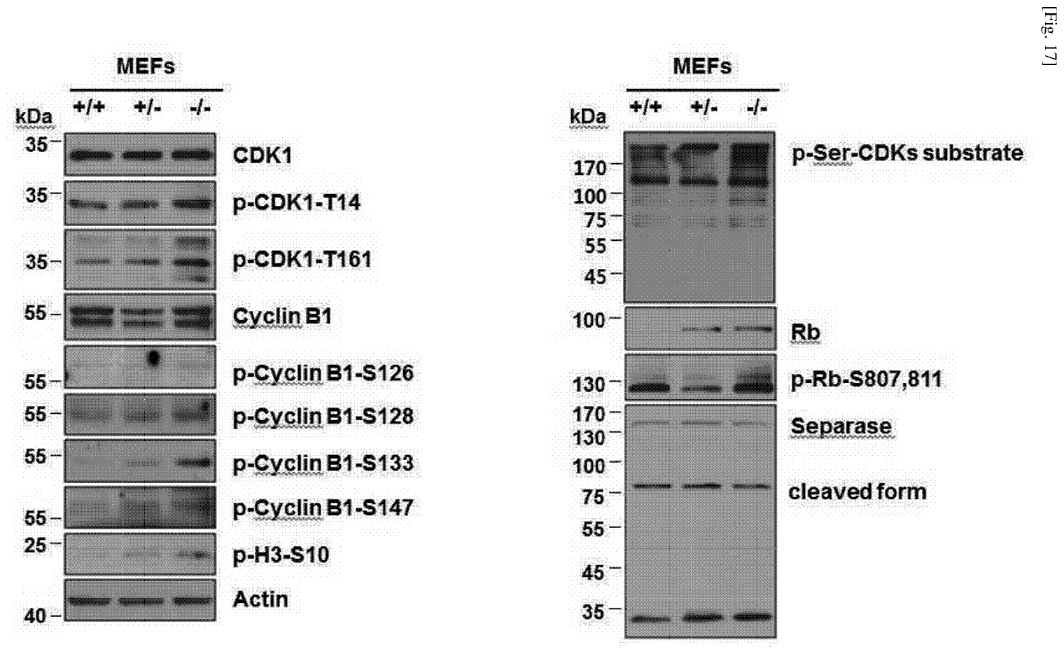

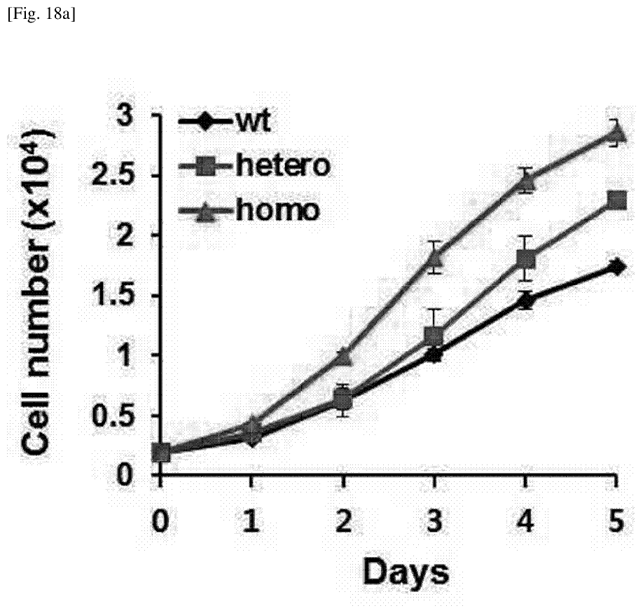

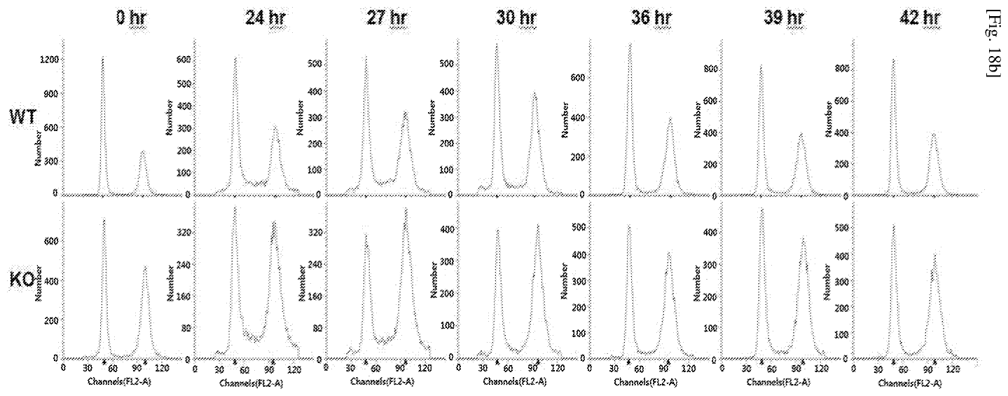

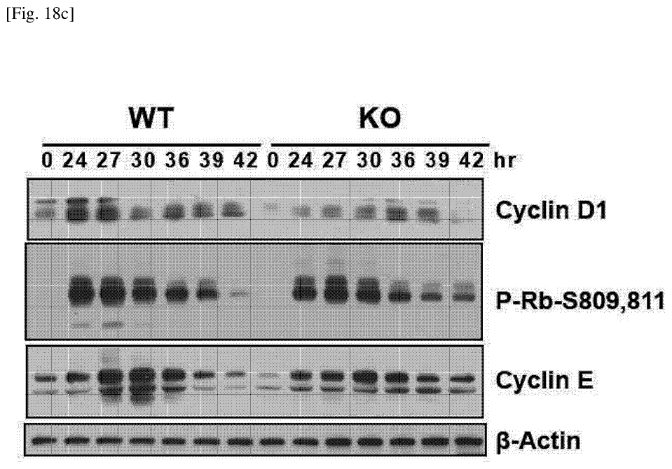

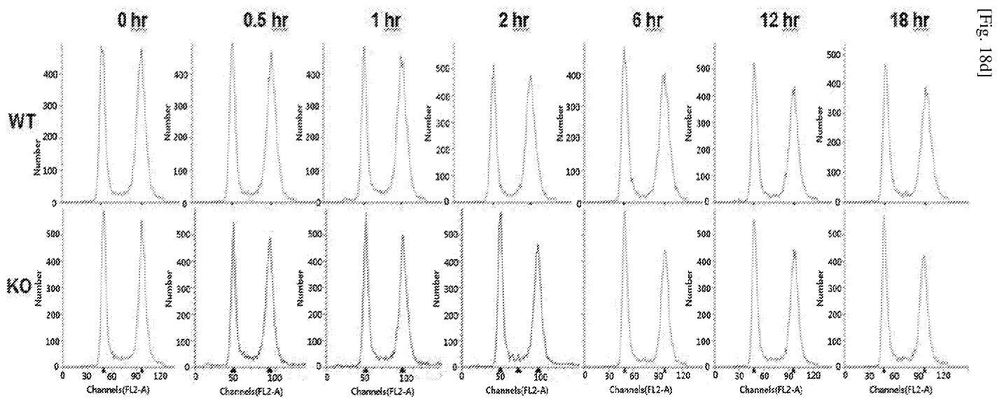

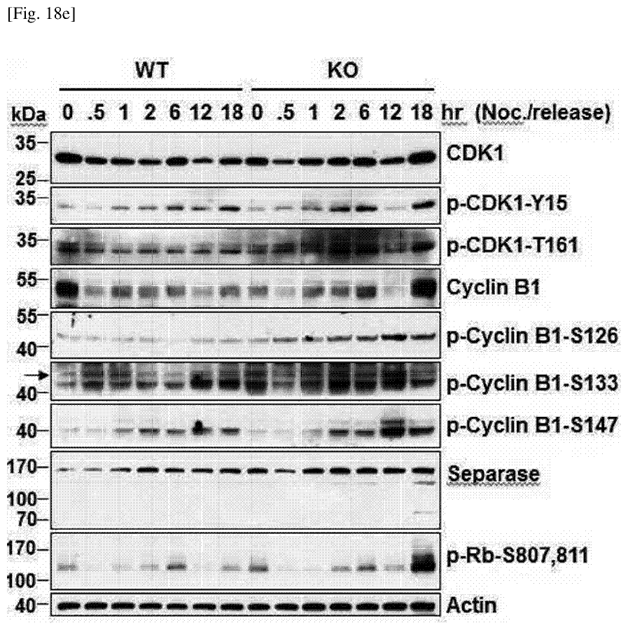

In another embodiment of the present invention, to confirm that the formation of a CDK1-Cyclin B1 complex and the activation thereof facilitate tumorigenesis in Rarres1 KO mice, as a result of preparing wild type and Rarres1-deficient MEFs according to Example 8-3 from embryos for in vitro culture on embryonic day 13.5 and identifying them through western blotting, it was confirmed that the phosphorylation of threonine 14 and 161 residues of CDK1 was enhanced in KO MEFs, the Rb protein was increased in KO MEFs, and separase, which is another substrate of CDK1, was cleaved in KO cells, which indicates that the activity of CDK1-Cyclin B1 is exhibited due to the deletion of Rarres1 (see FIG. 17). In addition, to examine whether an increase in the activity of CDK1-Cyclin B1 affects the growth of MEF cells, as a result of seeding the MEF cells on a 6-well plate, and counting the number of the MEF cells every day for 5 days, it was confirmed that Rarres1^{-/-} MEFs were rapidly grown (see FIG. 18A), and to evaluate that the improved tumor cell proliferation, which was observed in Rarres1^{-/-} MEFs, was induced by progression of a changed cell cycle, as a result of conducting a cell synchronization experiment according to Example 8-1, it was confirmed that Rarres1^{-/-} cells progressed more rapidly from the G1 to G2/M phases of the cell cycle (see FIG. 18B), and it was confirmed that while the expression of Cyclin E peaked at 30 hours after serum stimulation and was rapidly reduced in WT cells, Cyclin E peaked at 30 hours and was slightly reduced in KO cells (see FIG. 18C). In addition, it was confirmed through a nocodazole-release method coupled with FACS and western blotting analyses that mitotic exit was fast in Rarres1-null cells compared to WT cells (see FIG. 18D), and the phosphorylation of Rb, which is a substrate of CDK1, was increased in KO cells from 6 hours to 18 hours after release (see FIG. 18E). Taken altogether, it was confirmed that the activity of CDK1-Cyclin B1 was high in Rarres1 KO MEFs, and when compared to WT MEFs, the timing of tumor cell cycle progression was inadequate and rapid (see Example 12).

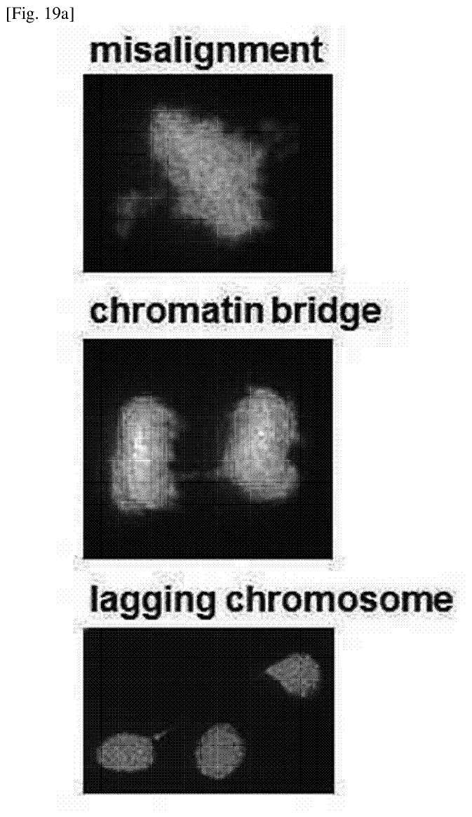

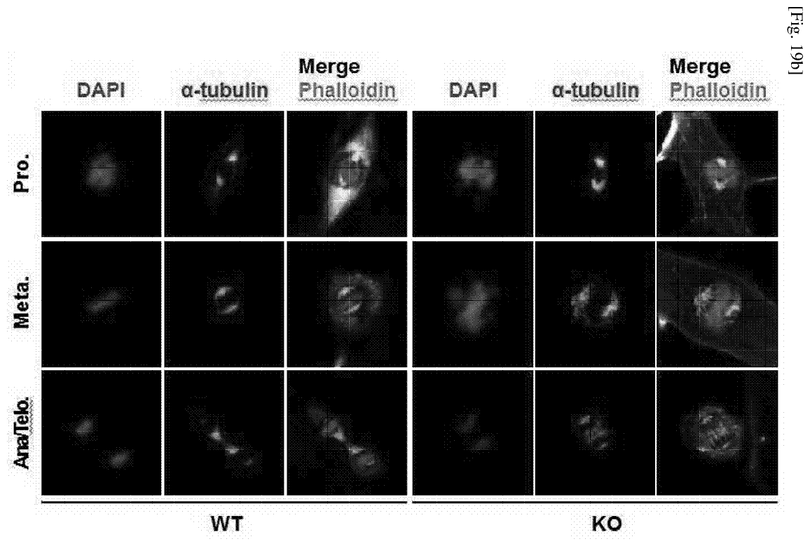

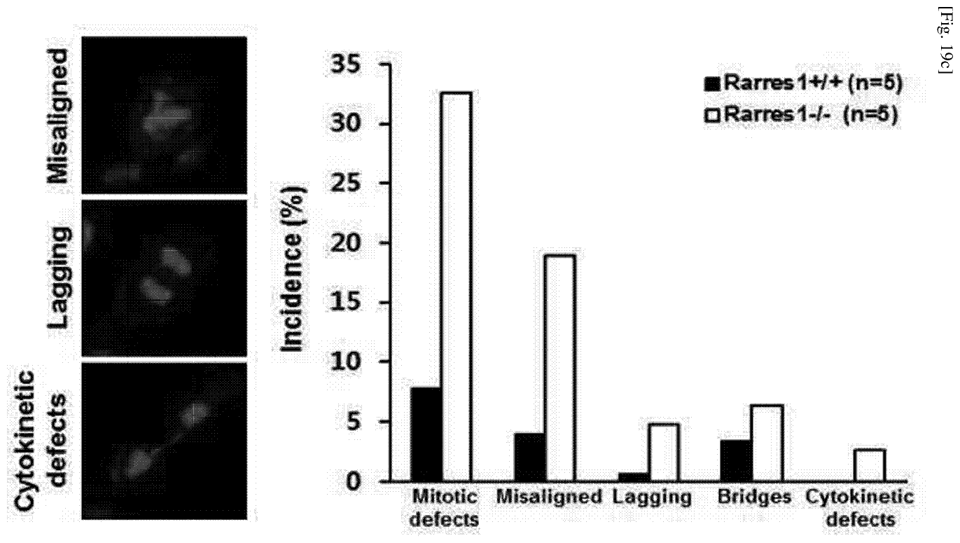

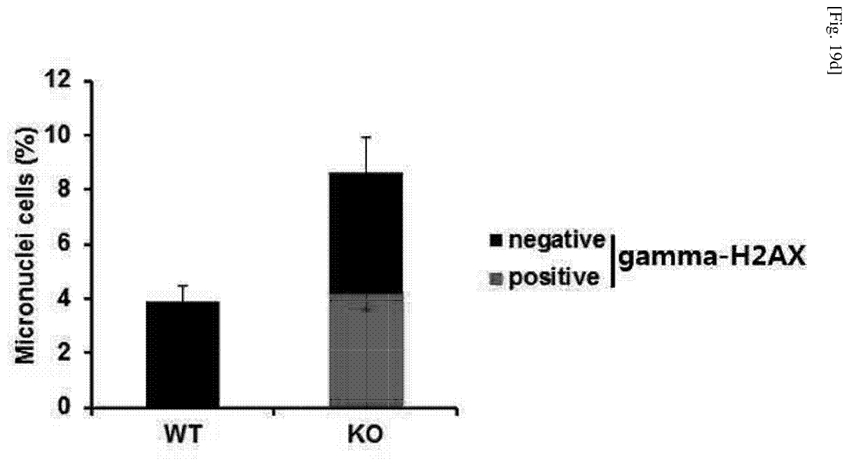

In another embodiment of the present invention, to confirm whether mitotic defects occur in Rarres1-deficient cells, as a result of observing several types of mitotic errors, it was confirmed that chromosome misalignment and missegregation occurred in Rarres1 KO MEFs during mitosis of the KO cells (see FIGS. 19A and 19B), and to confirm whether knockdown of Rarres1 affects chromosomal stability, as a result of analyzing metaphase spreads according to Examples 8 to 11 of Rarres1^{+/+} and Rarres1^{-/-} MEFs cultured with colcemide according to Example 8-7, it was confirmed that 20% or more of metaphase Rarres1^{-/-} MEFs exhibited substantial aneuploidy or polyploidy, and considerable DNA damage was confirmed when measured by damage-dependent phosphorylation of histone variant H2AX (H2AX foci formation) both in the micronuclei and primary nuclei of Rarres1-deficient cells, and thus it was confirmed that an increase in chromosome missegregation was associated with the occurrence of DNA damage foci and aneuploidy in the Rarres1-deficient cells (see Example 13).

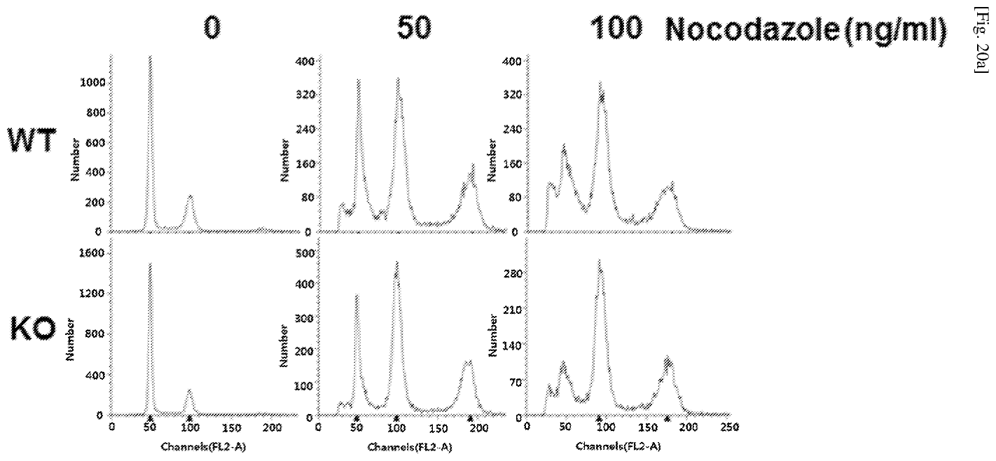

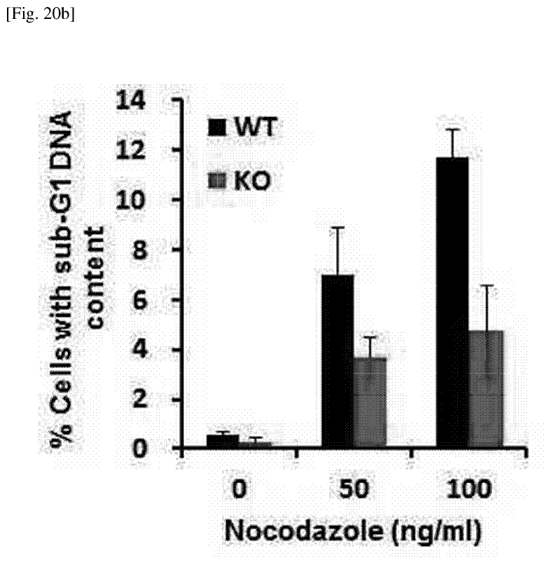

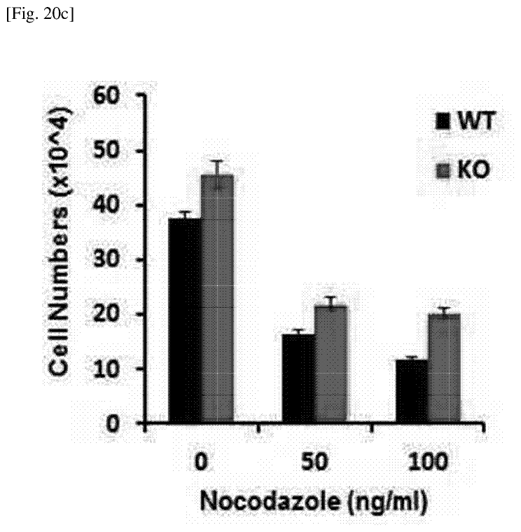

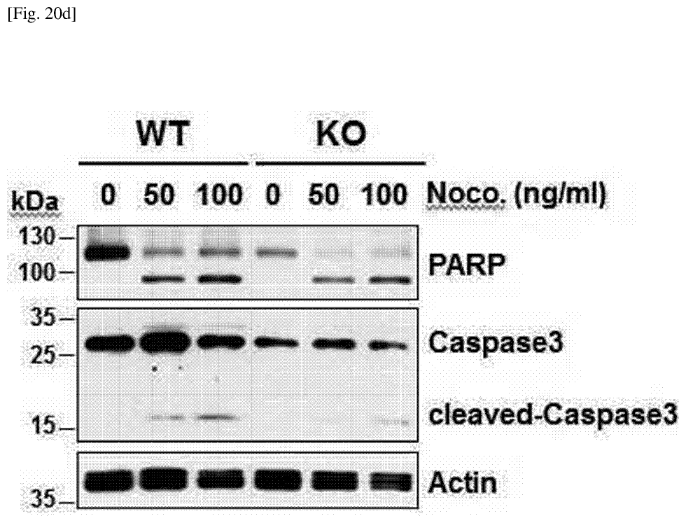

In another embodiment of the present invention, to confirm the effect of a mitotic stress-inducing drug such as nocodazole on Rarres1 KO MEFs, as a result of treating MEF cells with nocodazole or DMSO and performing flow cytometry and cell counting thereon according to Example 1-10, it was confirmed that cell death induced by nocodazole was significantly reduced in Rarres1-null MEFs in a dose-dependent manner (see FIGS. 20A and 20B), and the number of the Rarres1-null MEFs was less reduced by treatment with nocodazole, as compared to that of WT MEFs (see FIG. 20C). Consistent with these results, it was confirmed that the cleavage of PARP and Caspase 3 was decreased in KO cells compared to WT cells (see FIG. 20D), which indicates that the loss of Rarres1 causes resistance to nocodazole treatment (see Example 14).

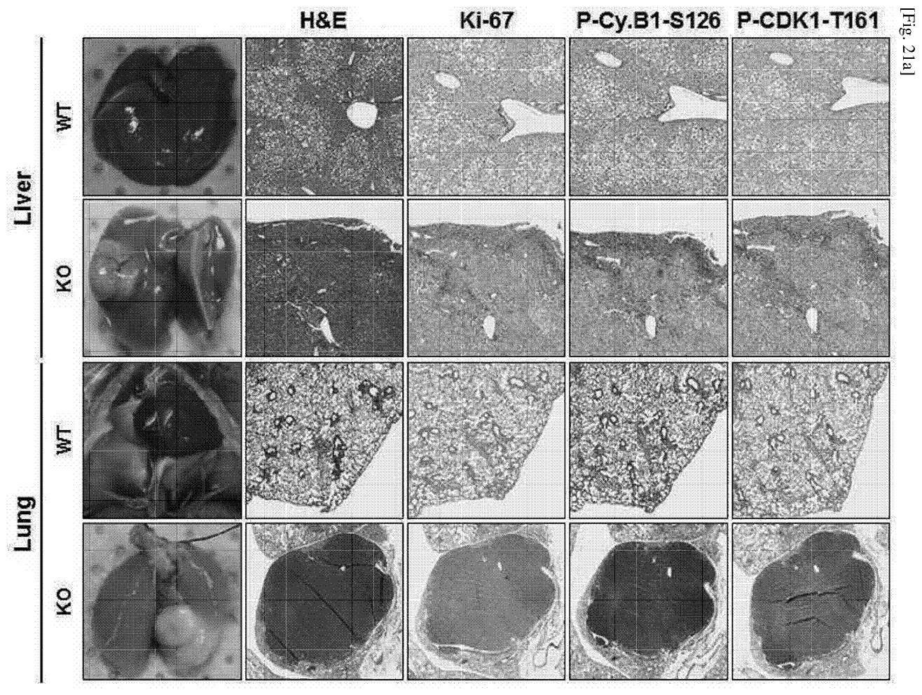

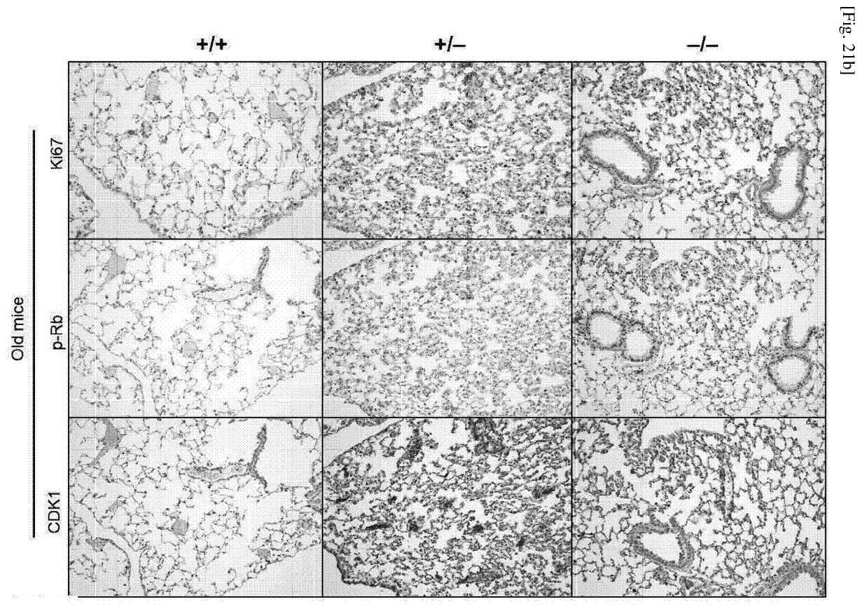

In another embodiment of the present invention, as a result of performing immunohistochemical staining according to Example 8-8 on phospho-Cyclin B1-ser126 and phospho-CDK1-T161 in liver cancer and lung cancer occurring in Rarres1 KO mice, it was confirmed that tumor sections of Rarres1-null mice were strongly stained with phospho-Cyclin B1-ser126 and phospho-CDK1-T161 while being stained negatively against the two antibodies around cancer cells (see FIG. 21A), which indicates that the activity of CDK1-Cyclin B1 is associated with tumorigenesis in Rarres1-null mice (see Example 15).

Therefore, the present invention provides a tumorigenic Rarres1^{+/N} chimeric animal model produced by injecting, into a blastocyst, an animal cell for producing a tumorigenic animal model, which is transfected with a retinoic acid receptor responder 1 (Rarres1) targeting vector for producing a tumorigenic animal model, the targeting vector including a DNA sequence consisting of, in the following order, a first locus of X-over P1 (loxP) site; a drug resistance gene region; a gene fragment including exon 3 of the Rarres1 genomic gene; and a second loxP site.

The targeting vector may further include, in the front of the first locus of X-over P1 (loxP) site, a DNA sequence consisting of, in the following order, a splicing acceptor (SA), β-galactosidase (β gal), and an SV40 polyA signal (pA), but the present invention is not limited thereto, and the drug resistance gene region may be, but is not limited to, a neomycin resistance gene.

In the targeting vector, all or part of the Rarres1 gene is floxed in a mammal except for a human, especially a mouse, according to the above-described DNA order. The floxed Rarres1 may be knocked out by deletion, translocation, or the like in a manner that allows Cre recombinase to be expressed in a transgenic animal. In addition, Rarres1 of a transgenic animal that tissue-specifically expresses Cre recombinase may be tissue-specifically knocked out.

A Cre-loxP system using the Cre recombinase and loxP is a gene knockout system and is a known method that causes a mutation in a target gene by inserting a loxP site into a target gene and expressing the Cre recombinase. When the target gene is floxed, two loxP sites are inserted into the target gene, e.g., an intron site or therearound, at a regular interval therebetween, and recombination occurs between the loxP sites in the presence of a Cre enzyme, resulting in deletion or translocation of a gene therebetween. In the present invention, first and second loxP sites may flank all or part of the Rarres1 gene, and as a result, all or part of the Rarres1 gene is deleted in the presence of Cre. In the absence of Cre, the floxed Rarres1 is not affected.

pCMVβ, which contains an intron including a splicing donor/splicing acceptor (SA) and polyadenylation signal (pA) derived from the SV40, and a full-length E. coli β-galactosidase (β gal) gene with eukaryotic translation initiation signals, is a mammalian reporter vector designed to express β-galactosidase in mammalian cells from the human cytomegalovirus immediate early gene promoter. pCMVβ expresses a high level of β-galactosidase and may be used as a reference plasmid when transfecting other reporter gene constructs, and may be used to optimize transfection protocols by using standard assays or staining to analyze β-galactosidase activity. Alternatively, the β-galactosidase gene may be excised using the Not I site at each terminal to allow other genes to be inserted into the pCMVβ vector backbone for expression in mammalian cells or to insert a β-galactosidase fragment into another expression vector, but the present invention is not limited thereto.

Since cells are unable to survive in an environment treated with neomycin when there is no neomycin resistance gene, the neomycin resistance gene is capable of filtering cells into which vehicles are not inserted, but the present invention is not limited thereto.

The animal cell may be preferably an embryonic stem cell (ES cell), and the ES cell may be generally obtained from preimplantation embryos cultured in vitro. The ES cell may be cultured using a method known in the art. Transfection of the targeting vector into the ES cell may be performed using a method known in the art. Examples of the method include pronuclear microinjection, retrovirus-mediated gene transfer, gene targeting, electroporation, sperm-mediated gene transfer, calcium phosphate/DNA coprecipitation method, microinjection, and the like. After the transfection, embryonic stem cells are cultured in an antibiotic-containing selection medium and resistance-exhibiting embryonic stem cell clones are selected, thereby selecting only homologous recombinant cells. In one embodiment of the present invention, loxPs are recombined with each other to cause the deletion of exon 3 therebetween, so that Rarres1 is knocked out. A transgenic animal for the expression of Cre recombinase may be produced. In this case, the Cre recombinase may be expressed only in all cells or specific tissue cells of the transgenic animal.

In the present invention, a chimeric animal model may be provided by injecting the animal cell into a blastocyst, and then implanting the injected animal cell in a surrogate mother, but the present invention is not limited thereto.

According to another embodiment of the present invention, there is also provided a tumorigenic Rarres1^{+/-} animal model produced by crossing the Rarres1^{+/N} chimeric animal model with an animal expressing Cre recombinase, wherein in the animal expressing Cre recombinase, a gene encoding the Cre recombinase is operably linked to a Zona pellucida 3 (Zp3) promoter, but the present invention is not limited thereto.

The term “zona pellucida 3” as used herein, which is also known as zona pellucida sperm-binding protein 3, zona pellucida glycoprotein 3, or a sperm receptor, refers to a ZP module-containing protein encoded by the ZP3 gene in humans. ZP3 is a zona pellucida receptor that binds sperm at the beginning of fertilization, but the present invention is not limited thereto.

According to another embodiment of the present invention, there is also provided a method of producing a tumorigenic Rarres1^{-/-} animal model, including: (a) producing the Rarres1^{+/N} chimeric animal model; (b) producing a Rarres1^{+/-} animal model through crossing of the chimeric animal model of process (a); and (c) selecting a Rarres1^{-/-} animal model from among progenies obtained by crossing the Rarres1^{+/-} animal model of process (b).

According to another embodiment of the present invention, there is also provided a tumorigenic Rarres1^{-/-} animal model produced by the above-described production method.

The animal model may have a tumor induced by the deletion of Rarres1, may induce mitotic defects or resist mitotic stress, may induce a somatic mutation, and may overexpress a gene such as Ccnd1, Cdkn1a, Cdkn2A, Nanog, Psrc1, Nup214, or the like in the mitotic cell cycle, but the present invention is not limited thereto.

In addition, the animal model may be produced using a mammal except for humans, and the mammal except for humans may be a monkey, a rat, a mouse, a rabbit, a dog, a non-human primate, or the like, and preferably may be an animal of the Muridae family, but the present invention is not limited thereto.

According to another embodiment of the present invention, there is also provided a method of screening for a tumor therapeutic agent, including: (a) treating a sample of a tumorigenic Rarres1^{-/-} animal model with candidate materials; (b) measuring phosphorylation levels of Cyclin-dependent kinase 1 (Cdk1) and Cyclin B1 of the sample, measuring amounts and activities of the CDK1 protein and the Cyclin B1 protein, measuring the expression or activity of Mist1 and LGR5, or measuring the activity of surfactant protein C (SPC)-positive cells; and selecting, as a tumor therapeutic agent, a candidate material exhibiting a decrease in phosphorylation levels of CDK1 and Cyclin B1, a candidate material exhibiting a decrease in amounts or activities of the CDK1 protein and the Cyclin B1 protein, a candidate material exhibiting a decrease in expression or activity of muscle, intestine and stomach expression 1 (Mist1) and leucine-rich repeat-containing G-protein coupled receptor 5 (LGR5), or a candidate material exhibiting a decrease in activity of surfactant protein (SPC)-positive cells, as compared to that in a group not treated with the candidate materials.

The present invention also provides a method of screening for a cancer therapeutic agent, including: (a) treating a sample with candidate materials in vitro; (b) measuring a degree of binding between Cyclin-dependent kinase 1 (CDK1) and Cyclin B1 of the sample; and (c) selecting, as a cancer therapeutic agent, a candidate material exhibiting a decrease in the degree of binding between CDK1 and Cyclin B1, as compared to that in a group not treated with the candidate materials.

The method may further include, in process (b), measuring a degree of binding between retinoic acid receptor responder 1 (RARRES1) and CDK1 or Cyclin B1 of the sample; and, in process (c), selecting, as a cancer therapeutic agent, a candidate material exhibiting a decrease in the degree of binding between CDK1 and Cyclin B1 and an increase in the degree of binding between RARRES1 and CDK1 or Cyclin B1, but the present invention is not limited thereto. Ultimately, process (c) is characterized in that RARRES1 binds to CDK1 or Cyclin B1 to inhibit the formation of a CDK1-Cyclin B1 complex, but the present invention is not limited thereto.

In addition, in process (b), the measuring may be performed by polymerase chain reaction (PCR), microarray, northern blotting, western blotting, enzyme-linked immunosorbent assay (ELISA), immunoprecipitation, immunohistochemistry, or immunofluorescence, but the present invention is not limited thereto.

In addition, the sample may be isolated from one or more selected from the group consisting of patients with spleen cancer, thymus cancer, liver cancer, lung cancer, renal cancer, thyroid cancer, small intestine cancer, stomach cancer, uterine cancer, and myeloid leukemia, but the present invention is not limited thereto. The sample may be, but is not limited to, one or more selected from the group consisting of tissue, a cell, whole blood, blood, saliva, sputum, cerebrospinal fluid, and urine.

In addition, the decrease in the degree of binding between CDK1 and Cyclin B1 is characterized by the inhibition of phosphorylation of serine 126 of the Cyclin B1 protein, but the present invention is not limited thereto, and the increase in the degree of binding between RARRES1 and CDK1 is characterized by binding to inactivated CDK1 at a C-terminal portion containing amino acids 251 to 294 of the RARRES1 protein, but the present invention is not limited thereto.

In addition, the candidate materials refer to unknown materials used in screening in order to measure the degree of phosphorylation, unknown materials used to measure the amounts or activities of the CDK1 and Cyclin B1 proteins, binding between CDK1 and Cyclin B1, unknown materials used to measure the expression or activity of Mist1 and LGR5, unknown materials used to measure the activity of surfactant protein (SPC)-positive cells, or unknown materials used in screening for measuring a degree of binding between CDK1 and Cyclin B1 or a degree of binding between RARRES1 and CDK1 or Cyclin B1, and preferably may be one or more selected from the group consisting of a compound, a microorganism culture or extract, a natural extract, a nucleic acid, and a peptide, but the present invention is not limited thereto. The nucleic acids may be one or more selected from the group consisting of an aptamer, a locked nucleic acid (LNA), a peptide nucleic acid (PNA), and a morpholino, but the present invention is not limited thereto.

In addition, measurement of a degree of phosphorylation of the sample, measurement of the amounts or activities of the CDK1 and Cyclin B1 proteins, measurement of the expression or activity of Mist1 and LGR5, measurement of the expression or activity of surfactant protein C (SPC)-positive cells, or measurement of a degree of binding between CDK1 and Cyclin B1 of the sample or a degree of binding between RARRES1 and CDK1 or Cyclin B1 may be performed by PCR, microarray, northern blotting, western blotting, ELISA, immunoprecipitation, immunohistochemistry, or immunofluorescence, but the present invention is not limited thereto.

In addition, the tumor may be selected from the group consisting of spleen cancer, thymus cancer, liver cancer, lung cancer, renal cancer, thyroid cancer, small intestine cancer, stomach cancer, uterine cancer, and myeloid leukemia, but the present invention is not limited thereto.

In addition, the Mist1, LGR5, or SPC, which is a stem cell marker, may be preferably a mouse-derived stem cell marker. In addition, the Mist1, LGR5, or SPC may be isolated from various organs, and preferably, Mist1 or LGR5 are markers for a stem cell in the stomach of a mouse, and SPC is a marker for a stem cell in the lung of a mouse, but the present invention is not limited thereto.

Hereinafter, exemplary embodiments will be described to aid in understanding of the present invention. However, the following examples are provided only to facilitate the understanding of the present invention and are not intended to limit the scope of the present invention.

### MODE FOR THE INVENTION

### Examples

### Example 1. Experimental Preparation and Experimental Methods

1-1. Cells and Transfection

Human mammalian epithelial cells were cultured in a mammalian epithelial cell medium (ScienCell Research Laboratories, Carlsbad, Calif., USA) supplemented with 10% (v/v) fetal bovine serum (FBS), 10,000 IU/ml of penicillin, 10,000 μg/ml of streptomycin, and a mammalian epithelial cell growth supplement (ScienCell). All other cells were grown in a medium supplemented with 10% (v/v) fetal bovine serum (FBS), 10,000 IU/ml of penicillin, 10,000 μg/ml of streptomycin, and sodium pyruvate at 37° C. in a humidified environment consisting of 95% air and 5% CO₂. For transfection with plasmid DNA according to Example 1-3 or siRNA according to Example 1-4, Lipofectamine LTX/PLUS (Invitrogen, Carlsbad, USA) and Lipofectamine2000 (Invitrogen) were respectively used according to the manufacturers' instructions.

1-2. RT-PCR

Total RNA was extracted from human cancer cell lines including prostate cancer, lung cancer, and breast cancer by using a TRIzol reagent (Invitrogen) according to the manufacturer's instructions, and 1 ug of total RNA was converted into cDNA with a Superscript® reverse transcriptase (Invitrogen). Primers used for human DNA were as follows: RARRES1-1-F (CGCATTCACTTGGTCTGGTA), RARRES1-1-R (CTGAAACCCTGAGGAACCTG), RARRES1-2-F (TTTGGGGAAATGTTCTGCTCG), RARRES1-2-R (CCACTTTGATTGTAACTCTTGTGG), Cyclin B1-F (CGGGAAGTCACTGGAAACAT), Cyclin B1-R (GATGCTCTCCGAAGGAAGTG), Actin-F (CATCGAGCACGGCATCGTCA), and Actin-R (TAGCACAGCCTGGATAGCAAC), and the primers respectively yielded PCR products with predicted sizes of 327 bp, 359 bp, 347 bp, and 626 bp. Primers used for mouse DNA were as follows: Rarres1-F(GCGCTGCACTTCTTCAACTT), Rarres1-R (GCCATAGCTGATGCTTCCAT), Gapdh-F (TGCACCACCAACTGCTTA), and Gapdh-R (GGATGCAGGGATGATGTTC), and these primers respectively yielded PCR products with predicted sizes of 653 bp and 177 bp.

1-3. Plasmids and Lentivirus

Wild-type RARRES1 isoform cDNA inserts were subcloned into the pcDNA3.1 expression vector (Invitrogen) respectively using BamH/EcoRV and Hind/EcoR restriction enzyme sites. The RARRES1 fluorescence expression vectors were constructed using the pAcGFP-C1 vector (Clontech, Heidelberg, Germany) with Sac/BamH restriction enzyme sites. RARRES1 deletion mutants were obtained by sequentially deleting 50 amino acids of wild-type RARRES1 sub-cloned into the pcDNA3.1 vector from the C-terminal thereof by PCR.

GST-Cyclin B1 and GST-Cdk1 were provided by Ju-Bac Park (Sungkyunkwan University, Suwon, Korea). All sequences were identified by automatic DNA sequencing. PmRFP-H2B was cloned into a GFP-deficient pLL3.1 lentiviral vector.

1-4. siRNA

For RARRES1 knockdown, cells were transfected with siRNA specific to RARRES1. According to Genbank Accession NM_206963 and NM_002888, siRNA duplexes may knock down both RARRES1 isoforms 1 and 2 (5′-AUGUUCUGCUCGAGUGUUU-3′), and are specific to RARRES1 isoform 1 (5′-AAUG AUGGUCUCAUCUCUGAA-3′) and RARRES1 isoform 2 (5′-GAGUUAC AAUCAAAGUGGU-3). As a control siRNA, 5′-GTTCAGCGTGTCCGGCGAG-3′ was used.

1-5. Cell Proliferation

For MTT assay, MDA-MB-231 and JIMT-1 cells were transfected with isoform 1 or 2 subcloned into pcDNA3.1, an empty vector, or RARRES1 by using the siRNA of Example 1-4, according to the method of Example 1-1. The cells were treated with a 3-(4,5-dimethylthiazol-2-yl)-2,5-diphenyltetrazolium bromide (MTT) solution for 2 hours, and then dissolved with DMSO, and the optical density (OD) of each sample was measured at 560 nm using an ELISA reader (Molecular Devices ELISA Reader, Sunnyvale, USA).

1-6. Live Cell Imaging

HEK293 Cells were maintained in DMEM containing 10% FBS and placed in a humidified incubator at 37° C. and 5% CO₂ inside a video microscope platform. Fluorescent images were captured every 10 minutes using a microscope (Carl Zeiss, Germany).

1-7. Cell Synchronization (DTB; Serum Starvation)

In the case of a double thymidine block (DTB) synchronized at the G1/S boundary, HEK293 cells were incubated with 2 mM thymidine for 16 hours and maintained in a normal medium for 10 hours, and then 2 mM thymidine was further added thereto for 16 hours.

1-8. Immunoblotting and Co-Immunoprecipitation

The cells were lysed in a lysis buffer (20 mM Tris, pH 7.4, 5 mM EDTA, 10 mM Na₄ P₂O₇, 100 mM NaF, 1% NP-40, 1 mM PMSF, 0.2% protease inhibitor cocktail and phosphatase inhibitor). The protein concentrations of the cell lysates were measured using a Pierce BCA Protein Assay kit (Pierce, Rockford, USA). Subsequently, protein lysates were resuspended in a loading buffer, boiled for 5 minutes, and then subjected to SDS-PAGE and immunoblotting with the indicated antibodies. For immunoprecipitation, a TAP buffer (25 mM Tris, pH 7.4, 140 mM NaCl, 0.5% NP-40, 10 mM NaF, 1 mM dithiothreitol (DTT), 1 mM phenylmethylsulphonyl fluoride, 1 mM EDTA, 1 mM Na₃VO₄, 1 mM β-glycerophosphate, 10% glycerol and 0.2% protease inhibitor cocktail and phosphatase inhibitor) was added to the cell lysates, and then the cell lysates were incubated along with a RARRES1 antibody (R & D System, MN, USA) or GST beads at 4° C. Protein expression was detected by chemiluminescence using the SuperSignal West Pico Chemiluminescent Substrate (Pierce).

1-9. Antibodies and Reagents

The following primary antibodies were used for immunoblotting according to Example 1-8, co-immunoprecipitation according to Example 1-8, and indirect immunofluorescence: Mouse monoclonal antibodies against Cdc2/cdk1 (Santa Cruz, sc-54, 1/1000), cdk1-phospho-tyr 15 (BD, 612306, 1/1000), Cyclin B1 (Cell Signaling, #4135, 1/2000), Cyclin D1 (Santa Cruz, sc-246, 1/1000), Rb (Cell Signaling, #9309, 1/2000), histone H3-phospho-ser 10 (Abcam, ab14955, 1/500), p62 (BD, 610832, 1/1000), γH2AX (Millipore, #05-636, 1/500), β-actin, and α-tubulin (Sigma-Aldrich, 1/5000), as well as rabbit polyclonal antibodies against CDK1-phospho-Thr 14 (Abgent, AP7517d, 1/500), Cdk1-phospho-Thr 161 (Cell Signaling, #9114, 1/1000), phosphor-(ser) CDKs substrate (Cell Signaling, #2324, 1/1000), Wee1 (Cell Signaling, #4936, 1/1000), Cdk2 (Cell Signaling, #2546, 1/1000), Cyclin B1 (Cell Signaling, #4138, 1/1000), Cyclin B1-phospho-ser 126 (Abcam, ab55184, 1/1000), Cyclin B1-phospho-ser 128 (Santa Cruz, sc-130591, 1/1000), Cyclin B1-phospho-ser 133 (Cell Signaling, #4133, 1/1000), Cyclin B1-phospho-ser 147 (Cell Signaling, #4131, 1/1000), Cyclin E (Santa Cruz, sc-481, 1/1000), Cyclin A (Santa Cruz, sc-751, 1/1000), Aurora A (Cell Signaling, #3092, 1/1000), Aurora B (Cell Signaling, #3094, 1/1000), Rb-phospho-ser 807,811 (Cell Signaling, #9308, 1/1000), GST (Abfrontier, LF-PA0189, 1/1000), Cdk4 (Santa Cruz, sc-260, 1/1000), PARP (Cell Signaling, #9542, 1/1000), caspase3 (Cell Signaling, #9602, 1/1000), Ki-67 (Abcam, ab15580, 1/3000), and a goat antibody against RARRES1 (R&D Systems, AF4255, 1/1000). Alexa Flour 488 phalloidin (Invitrogen) was used for F-actin staining. Horseradish peroxidase-tagged secondary antibodies (Jackson Immuno Research Laboratories, Inc., West Grove, USA) were used. Bafilomycin A1 (Selleckchem, S1413), E-64-D (Enzo, BML-PI107), Pepstatin A (Sigma-Aldrich, P5318) and MG132 (Calbiochem, 474790) were used as protein degradation inhibitors. Other reagents used in this study were purchased from Sigma-Aldrich (St. Louis, USA) unless stated otherwise. All reagents were used in accordance with the manufacturer's recommended protocol.

1-10. Flow Cytometry

For analysis of cell cycles or sub-G1 DNA contents, cells were immobilized with 80% ice-cold ethanol, and stored at 4° C. The cells were stained with PBS containing 50 ug/ml of propidium iodide (PI) and 100 ug/ml of RNase A at 37° C. for 30 minutes. DNA content was analyzed by FACS Calibur flow cytometry and the results thereof were analyzed using Cellquest software (Becton-Dickinson Immunocytometry Systems, San Diego, Calif.) and Modfit LT 3.3 software. At least 10,000 cells were analyzed per sample.

### Example 2. Inactivation of RARRES1 by Hypermethylation in Human Cancer Cell Lines

Expression levels of RARRES1 isoforms in human normal cells and cancer cell lines including prostate cancer, lung cancer, and breast cancer were evaluated by RT-PCR according to Example 1-2. The results thereof, which coincided with previously published data, showed that RARRES1 expression was silenced in most of the tested human cancers, when compared to the normal cells. RARRES1 expression was shown at a level higher than that in normal cells or other cancers in some of the cancer cells in the lung (1/6; Calu3) and the breast (3/11; MDA-MB-468, HCC70, and HCC1569). The mRNA expression patterns of both RARRES1 transcript variants were very similar in all the human cancer cell lines (see FIG. 1A to FIG. 1C).

To determine whether promoter methylation was associated with the silencing of the RARRES1 expression in cancer cells, cells were treated with 5-aza-2′-deoxycytidine (5-aza-2-dC; 5, 25, and 100 uM) as an inhibitor of DNA methylation for 5 days. 4/4 (100%), 4/5 (80%), and 8/10 (80%)) showed restored RARRES1 expression in a dose-dependent manner in most of the tested cell lines (prostate (see FIG. 2A), lung (see FIG. 2B), and breast (see FIG. 2C)) after treatment with 5-aza-2-dC, but the degrees of restoration were different.

However, cells having high mRNA levels of RARRES1 including Calu3 and HCC70 failed to have reintroduced RARRES1 expression by treatment with 5-aza-2-dC (see FIGS. 2B and 2C). Rather, the mRNA level of the RARRES1 gene was reduced in JIMT-1 cells (see FIG. 2C). Similarly, in terms of endogenous mRNA expression patterns, the two isoforms showed almost the same methylation in prostate cancer, lung cancer, and breast cancer. However, in some cancer cells, although isoform 1 was increased under conditions of treatment with 5-aza-2-dC, CWR22rv, T47D, isoform 2 were not detected in a demethylated state by 5-aza-2-dC. From these results, it was confirmed that the inhibition of RARRES1 in prostate, lung, and breast cancer cell lines was mediated at least in part by promoter methylation of the RARRES1 gene.

### Example 3. RARRES1 as Putative Tumor Suppressor Gene

Promoter hypermethylation may be a mechanism for inactivating tumor suppressor genes in cancer. As described in Example 2, it is assumed that RARRES1 acts as a tumor suppressor gene, based on the results of FIGS. 1A to 1C and 2A to 2C. To test this hypothesis, the effect of RARRES1 on cell proliferation according to Example 1-5 in breast cancer cell lines MDA-MB-231 and JIMT-1 measured by MTT cell proliferation assay was examined.

Both or each of RARRES1 transcript variants were specifically expressed in MDAMB-231 cells exhibiting a low mRNA level of RARRES1, and cell growth was analyzed for 5 days. Cancer cell growth was gradually inhibited for a certain period of time after transient transfection with both or each of RARRES1 isoform expression vectors according to the method of Example 1-1 (see FIG. 3A). In contrast, RARRES1 mRNA expression was reduced when JIMT-1 cells were transfected, using the method of Example 1-1, with a specific siRNA according to Example 1-4, against the RARRES1 variants, and cell viability according to Example 1-5 was measured by MTT assay for 5 days. Cell viability was enhanced in all RARRES1-deficient cells. Such an improved effect of RARRES1 on the cell proliferation according to Example 1-5 was greatest in the two variants and suppressed more than in each variant (see FIG. 3B). This data indicates that RARRES1 acts as a putative candidate tumor suppressor gene.

### Example 4. Mitotic Arrest Induced by RARRES1 Overexpression

As described in Example 3, RARRES1 negatively regulated cell proliferation in cell viability experiments. Since there is the possibility of an increase in apoptosis, flow cytometry (FACS) according to Example 1-10 was performed to confirm this when RARRES1 was overexpressed in MDA-MB-231 cells (see FIG. 4A) and HEK293 cells (FIG. 4B), and from the results thereof, it was confirmed that RARRES1 hardly induced apoptosis.

To evaluate whether the reduced cell growth observed in RARRES1-overexpressing cells was due to altered cell cycle progression, HEK293 cells were transfected with green fluorescent protein (GFP) tagged-RARRES1 or -empty vector (Ctrl) according to the method of Example 1-1, and the cells were observed by live cell imaging according to Example 1-6. From now on, all data shown was regarded as isoform 1 since there was no difference between the two isoforms of RARRES1 in the above experiments, and it was named ‘RARRES1.’ In HEK293 cells transfected with a GFP control (Ctrl) according to the method of Example 1-1, the cells underwent normal cell cycle progression. A cell entered into mitosis (round shape) within 1 hour, and was separated into two daughter cells within at least 2.5 hours (see FIG. 5A, top panel). However, GFP-tagged RARRES1-overexpressing 293 cells were present during mitosis for 2.5 hours, and were not separated into two daughter cells during monitoring (see FIG. 5A, bottom panel). To confirm mitotic arrest induced by RARRES1 overexpression, GFP-control or GFP-RARRES1-overexpressing 293 cells and 293 cells transfected with a red fluorescent protein (RFP) lentivirus used to label GFP non-transfected cell, according to Example 1-3 were co-cultured, and changes in the number of fluorescence-expressing cells (see FIG. 5B) were monitored. Up to 72 hours, the GFP-RARRES1-overexpressing cells had a round shape and were present for a longer period of time, whereas the number of RFP-expressing cells was gradually increased. Under co-culture conditions of GFP-control coupled with RFP virus-transfected cells, the numbers of both fluorescence-expressing cells were increased over time. Only the GFP-RARRES1-overexpressing 293 cells showed mitotic arrest and were not divided, and other cells normally progressed through the cell cycle (see FIG. 5C). From the double thymidine block (DTB) experiment according to Example 1-7, it was confirmed that RARRES1-overexpressing cells rapidly entered into the G2/M phases after 4 hours (21.38% for the control, and 29.85% for RARRES1), and the accumulation thereof in the G2/M phases was increased after 6 hours as compared to the control (77.41% and 80.91%, respectively) and persisted longer in the G2/M phases at 8 hours (45.47% for the control and 54.46% for RARRES1). 10 hours after release from DTB, both cells successfully returned to the G1 phase (see FIG. 5D). The RARRES1 protein was slightly increased at 2 hours, gradually increased up to 10 hours, and showed a peak at 12 hours after release. On the other hand, mRNA levels were present over time (see FIG. 5E). These results suggest that RARRES1 overexpression induces mitotic arrest in HEK293 cells.

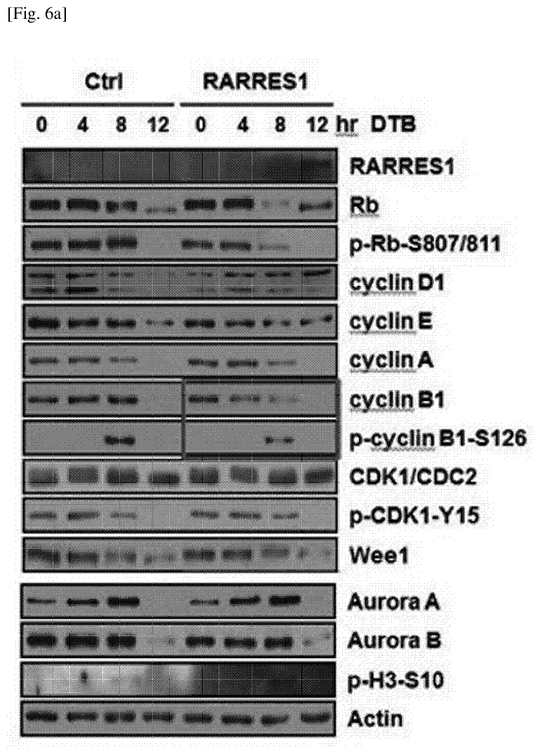

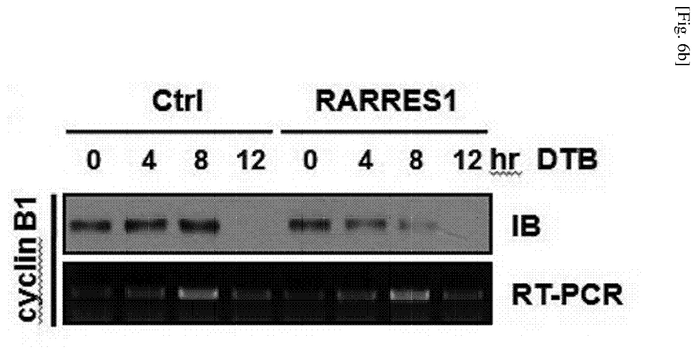

To investigate which cell cycle regulatory proteins were modified in RARRES1-overexpressing cells, western blotting analysis was performed using the DTB method according to Example 1-7. The Rb protein and Rb phosphorylation (ser 807,811) were decreased in RARRES1-overexpressing cells compared to those of control cells. In particular, the expression and phosphorylation (serine 126) of Cyclin B were decreased at 8 hours after release in the presence of RARRES1. The expression and inhibitory phosphorylation (tyrosine 15) of CDK1 did not change, and Wee1, which is a kinase that inhibits the phosphorylation of CDK1 at the tyrosine 15 residue of CDK1, also did not change (see FIG. 6A). Cyclin B1 mRNA was not changed in all of both transfected cells when measured by RT-PCR according to Example 1-2. mRNA expression was peaked at 8 hours after release (see FIG. 6B). Taken together, these results showed that the overexpression of RARRES1 in HEK293 cells was associated with the activity of Cyclin B1.

### Example 5. RARRES1 Inhibiting Formation of CDK1-Cyclin B1 Complex in Mitosis

To test whether RARRES1 affected Cyclin B1 activation during mitosis through direct binding, HEK293 cells were co-transfected with RARRES1 and glutathione S-transferase (GST)-tagged Cyclin B1 using Lipofectamine LTX/PLUS, according to the method of Example 1-1, and cell lysates were immunoprecipitated with GST beads according to Example 1-8. This shows that RARRES1 directly binds to Cyclin B1. Interestingly, Cyclin B1 less efficiently interacted with endogenous CDK1 in the presence of RARRES1 than in the absence of RARRES1. CDK1 expression was the same under the same conditions (see FIG. 7A). Next, it was examined how the presence of RARRES1 reduced the formation of the CDK1-Cyclin B1 complex. A GST pull-down assay was performed on 293 cells co-transfected with RARRES1 and GST-CDK1, according to the method of Example 1-1. CDK1 directly interacted with RARRES1 and reduced binding to endogenous Cyclin B1 in the presence of RARRES1. Relatively, Cyclin B1 expression was slightly reduced in the presence of RARRES1 rather than in the absence of RARRES1 (see FIG. 7B). To confirm the interaction of RARRES1 with Cyclin B1 or CDK1, mitotic (+nocodazole, 50 ng/ml) or exponentially growing (−nocodazole) 293 cells were precipitated with a RARRES1-specific antibody. Although endogenous CDK1 is associated with RARRES1 regardless of nocodazole treatment, endogenous Cyclin B1 (see FIG. 7C) predominantly binding to RARRES1 in mitosis suggests that RARRES1 is an endogenous inhibitor of the CDK1-Cyclin B1 complex in mitosis.

In addition, it was examined whether RARRES1 interacted with other CDK-Cyclin complexes, including interphase CDKs (CDK2, CDK4, and CDK6) and their binding partner Cyclins, and immunoprecipitation according to Example 1-8 was performed on 293 cells under the same conditions as those in FIG. 7C. Each component of CDK4-Cyclin D and CDK2-Cyclin A complexes did not bind to RARRES1 (see FIGS. 8A and 8B). These results suggest that RARRES1 specifically inhibits the formation of a CDK1-Cyclin B1 complex.

### Example 6. Searching for Amino Acid Region of RARRES1 Protein Binding to CDK1

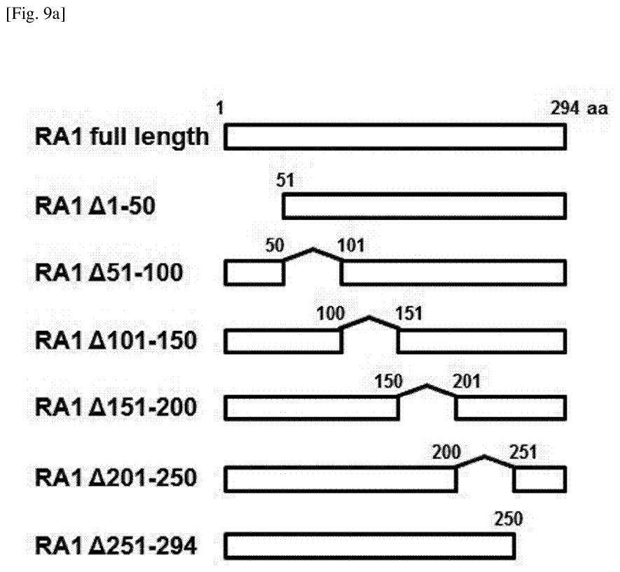

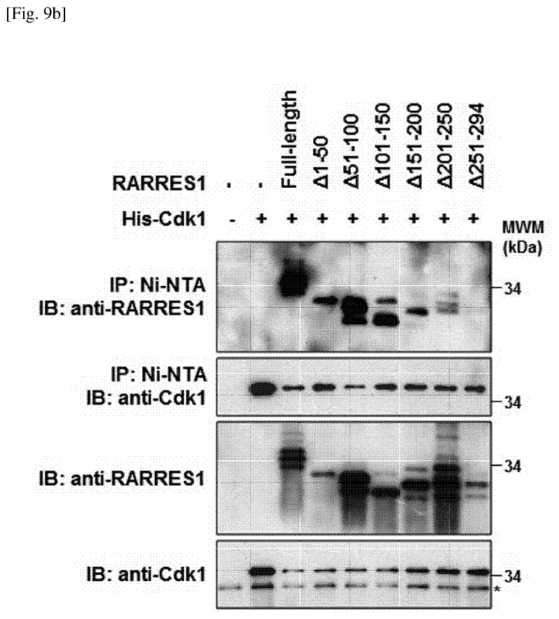

To find out which amino acid region of the RARRES1 protein directly binds to CDK1, RARRES1 protein mutants having sequences with different sequences of 50 deletions were prepared (see FIG. 9A), and then 293 cells transfected with His-CDK1 using the method of Example 1-1 were immunoprecipitated according to Example 1-8. Binding to CDK1 hardly occurred in mutants with the deletion of amino acids 251 to 294 at the C-terminal of RARRES1 (see FIG. 9B). These results suggest that the C-terminal of RARRES1 is a crucial region for CDK1 binding.

### Example 7. Degradation of CDK1 by RARRES1 Occurring in Lysosomes

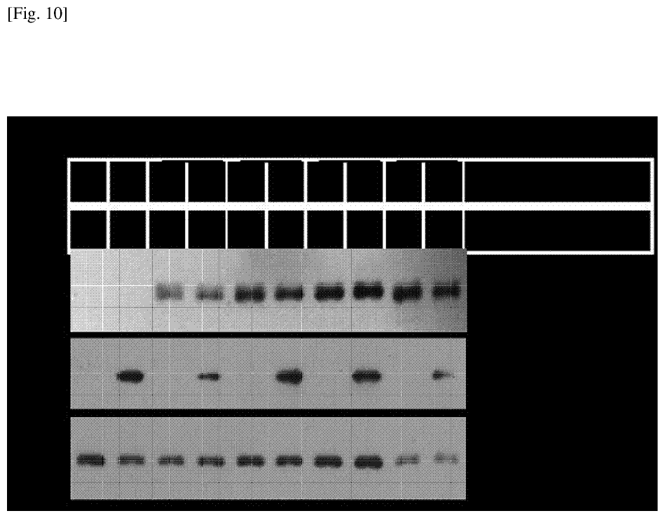

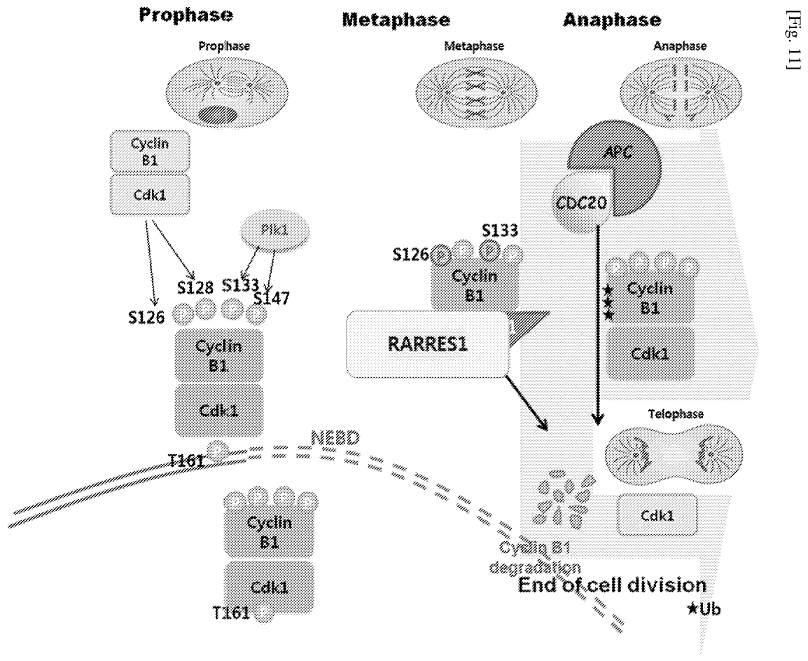

In addition, when cells were transfected with RARRES1 along with CDK1 using the method of Example 1-1, the amount of the CDK1 protein was reduced. To examine how a quantitative change in the CDK1 protein was regulated, cells were treated with protein degradation inhibitors. Upon co-treatment with E-64-D and Pepstatin A (E/P) or Bafilomycin (BafA1), which is an inhibitor of protein degradation by lysosomes, the amount of the CDK1 protein, which had been decreased by RARRES1, was increased again. In contrast, when cells were treated with MG132, which is an inhibitor of protein degradation by proteasomes, the CDK1 protein was still decreased by RARRES1 (see FIG. 10). Taken altogether, these results suggest that binding of RARRES1 to CDK1 through the C-terminal thereof causes instability of the CKD1 protein through lysosomes.

### Example 8: Experimental Preparation and Experimental Methods

8-1. Cell Synchronizations (Serum Starvation and Nocodazole Release)

To synchronize mouse embryo fibroblasts (MEFs) in the G0 phase, cells were washed four times with PBS and then cultured in a medium containing 0.1% fetal bovine serum (FBS) for 72 hours. Subsequently, the medium was replaced with a normal medium containing 10% FBS to allow the cell cycle to restart and then the cells were harvested at a specific time.

To monitor mitotic exit, nocodazole release was carried out. About 30% to about 40% confluent MEFs were cultured in a medium containing 80 ng/ml of nocodazole for 12 hours, and then the cells were washed four times with PBS and then the medium was replaced with a normal medium to allow their exit to mitosis, and then the cells were harvested at a specific time.

8-2. Generation of Rarres1 Knockout Mice

ES cell clones (F07 and A05) for Rarres1 knockout mice were obtained from a knock out mouse project (KOMP). β-galactosidase (β gal) and neomycin-resistant (neo) selection cassettes were inserted into intron 2 of the murine Rarres1 gene. Chimeras were generated through blastocyst injection of ES cells and germlined chimeras were backcrossed with the C57BL/6 strain to obtain mice heterozygous for Rarres1. All mice used had the C57BL/6 genetic background and housed in a pathogen-free barrier environment and maintained on a normal diet. Genotyping of embryos and mice was performed by PCR using primers KO-1F (CTGGGTTCTAGCCAGTTTACAGTT), Ex3-R (ACTCAGCTTTGGGTAGCATTAGTC), F4 (CAGTTGGTCTGGTGTCAAAAATAA), and KO-2R (CTCAGGTTCTAGACTTCCCTGAAA), and the primers yielded PCR products with predicted sizes of 593 bp (wild-type allele) and 478 bp (knockout allele).

8-3. Culture of Mouse Embryo Fibroblasts and Epithelial Cells

Mouse embryo fibroblasts (MEFs) and mouse embryo epithelial cells (MEEs) were derived from 13.5-day-old embryos as previously described. That is, after removal of the head and internal organs, embryos were rinsed with phosphate buffered saline (PBS), minced, and treated with trypsin/EDTA to obtain single cells. The MEFs was resuspended in DMEM containing 10% FBS, 2 mM L-glutamine, 0.1 mM MEM nonessential amino acids, 55 uM beta-mercaptoethanol, and 100 IU/ml penicillin, and 100 ug/ml streptomycin. Cell media and reagents were obtained from GIBCO (Paisley, England). The MEEs were cultured in a D-MEM/F-12 medium containing 1% FBS, 1 mg of insulin, 1 mg of hydrocortisone, 12.5 μg of EGF, 10 mg of ascorbic acid, 10 mg of transferin, 14.1 mg of phosphoethanolamine, Na selenite, 1 μg of cholera toxin, 6.5 μg of triiodo thyronine, 35 mg of bovine pituitary extract, ethanolamine, 50 IU/ml of penicillin, and 50 ug/ml of streptomycin. The cells were incubated at 37° C. in a 5% CO₂-humidified chamber. After disintegration of the embryos, plating was considered passage 0. All experiments were carried out using cells within passage 5 from three different batches. The genotypes of the MEFs and MEEs were confirmed by genotyping PCR. At least three independently generated cell lines per genotype were used.

8-4. LacZ Staining of Mouse Embryos

To visualize the expression of Rarres1, the expression of LacZ was analyzed from the knockout allele. Embryos aged between embryonic day 11.5 (E11.5) and embryonic day 14.5 (E14.5) were fixed with 2% paraformaldehyde on ice overnight, washed with PBS, and cultured overnight in a staining solution (1 mg/ml of X-gal [5-bromo-4-chloro-3-indolyl-β-D-galactopyranoside], 1 mM potassium ferricyanide, and 1 mM potassium ferrocyanide in washing buffer). The embryos were washed three times (20 minutes each) with washing buffer (2 mM MgCl₂, 0.01% deoxycholate, 0.02% NP-40, 0.1 M sodium phosphate, pH 7.3), and re-fixed with 2% paraformaldehyde at 4° C. overnight, followed by dehydration with 70% ethanol.

8-5. PET/CT Imaging

All mice were fasted (fed only water) for at least 6 hours for PET/CT scanning. 18F-fluorodeoxyglucose (FDG) (370 MBq) was intravenously injected to the mice to obtain axial raw data on a PET scanner. The acquisition time was about 20 minutes. Axial images were reconstructed with a Shepp-Logan filter (cutoff frequency, 0.35 cycles per pixel) and realigned in coronal and sagittal planes. Spatial resolution was 6.1±6.1±4.3 mm.

8-6. Immunofluorescence

MEFs were fixed in PBS/3.7% paraformaldehyde for 10 minutes at room temperature (RT), permeabilized in 0.2% PBS/Triton X-100 for 5 minutes, and blocked in PBS/3% BSA for 30 minutes at room temperature. The samples were incubated overnight at 4° C. with the primary antibody. Then, the samples were washed three times with 0.05% Tween-20/PBS, incubated with secondary antibodies for 2 hours at RT and DAPI (0.5 ug/ml) for 5 minutes at RT, and then washed with PBS. Coverslips were mounted onto glass slides using a Prolong Gold antifade reagent (Invitrogen), followed by observation using a confocal microscope (Zeiss 510 Meta, Carl Zeiss). For quantification of gamma-H2AX-positive cells, at least 100 cells per each MEF line were analyzed.

8-7. Metaphase Spreading

To prepare metaphase spreads from MEFs and analyze them through aneuploidy, MEFs at passage 5 were treated with 0.1 ug/ml of colcemide (GIBCO BRL) at 37° C. for 4 hours to 5 hours. After trypsinization, the cells were centrifuged at 800 rpm for 10 minutes, resuspended in 5 ml of 0.075 M KCl, and incubated at 37° C. for 20 minutes. They were fixed in a freshly prepared Carnoy's solution (methanol:glacial acetic acid=3:1), and then resuspended in an appropriate amount of Carnoy's solution. The cell suspension was dropped onto microscope/glass slide and air dried. Chromosomes were visualized by DAPI (0.5 ug/ml) staining and analyzed under a confocal microscope (Zeiss 510 Meta, Carl Zeiss) using a 100× objective lens.

8-8. Immunohistochemistry (IHC)

Mice were sacrificed, and their organs were fixed in neutral-buffered formalin (10%) for 24 hours, and embedded in paraffin. Paraffin-embedded tissue blocks were cut to a thickness of 4 um and sections were dried at 65° C. for 1 hour. Immunohistochemical staining was performed using the automated staining instrument Discovery XT (Ventana Medical System, Inc. Tucson Medical System, Inc., Tucson Ariz., USA) as follows. Sections were deparaffinized and rehydrated with EZ prep (Ventana) and washed with reaction buffer (Ventana). Antigens were recovered through heat treatment in pH 6.0 citrate buffer (Ribo CC, Ventana) at 90° C. for 30 minutes for anti-phospho-Cyclin B1-ser 126, anti-phospho-CDK1-thr 161, and anti-Ki67.

8-9. Whole Genome Sequencing and Next-Generation Sequencing Analysis for RNA-Seq

WGS data was produced from genomic DNA of each sample, and the genomic DNA was amplified and sequenced using HiSeq 2500. Low quality reads were trimmed using Trimmomatic v.0.36, and the trimmed reads were aligned on a mm10 basis using BWA 0.7.13. Joint cleaning and post alignment quality control were performed using GATK 3.5 and Picard 2.7.1. MuTect2 was used to identify somatic mutations for coincident pairs of tumor samples and normal samples. When there were no coincident normal samples, variant calling was performed using each of three non-coincident normal samples as a control, and variants called at least twice were selected. All somatic mutations were filtered to eliminate germline variants, which are referred to as wild-type embryos, and annotated using ANNOVAR 2016.2.1. When all normal samples were used as normal panels in accordance with the GATK best practice, somatic copy number variants (CNVs) were detected. Somatic structural variants (SVs) were called using Manta v1.3.2. In the case of an incomparable tumor sample, SV calling was performed on each of three incomparable normal samples as a control, and an intersection was selected. In addition, RNA-Seq low-resolution reads were trimmed, and aligned with the mm10 and RefSeq gene model references using STAR 2.5.2b. Gene expression profiles were quantified using RSEM 1.3.0. To identify genes that significantly change over time in a knockout group compared to a wild-type group, testing for the following comparison was performed.

1. Three points of the knockout group (with 18-month-old tumor samples), 2. Three points of the knockout group (with 18-month-old normal samples), 3. Three points of a wild-type group, 4. 18-month-old tumor and normal samples.

The R package ‘EBSeqHMM’ was used to identify genes differentially expressed in the first three tests. The fourth test was conducted using the R package ‘limma.’ Among significant genes in the first test, genes that were not differentially expressed in the fourth test were filtered out, even though they exhibited statistically significant results in the second and third tests. Gene set enrichment analysis (GSEA) was performed on a selected set of genes by using DAVID 6.7, and pathway activity was evaluated by gene set variation analysis (GSVA) with reference to the MSigDB HALLMARK pathway gene set.

In addition, the results of differential expression analysis were integrated through a protein-protein interaction (PPI) network. First, the PPI network was constructed by adding experimentally proven protein interactions to the mouse BioGRID network. Based on a total p value calculated using p values adjusted in the first and fourth tests, an optimum subnetwork was derived from the PPI network using the R package ‘BioNet.’ Once again, GSEA was performed on genes of the optimum subnetwork.

8-10. Additional Function Research Using TCGA Lung Adenocarcinoma and Comparison with Single Cell Profile

Somatic mutations, CNVs, gene expression profiles, and related pathways of RARRES1 from TCGA lung adenocarcinoma were investigated. Somatic mutations and CNVs were derived from TCGA level3 data. Next, pathway activity for each cluster was estimated using GSVA, and the gene expression of major markers was examined from gene expression profiles. In addition, states of the CDK1 protein that binds to RARRES1 and CDK1 mRNA were examined from the comparison between RPPA and gene expression. The abundant states of normal and interstitial lung cells were estimated from the results of single cell mouse atlas (scMouse) lung tissue, and 24 normal lung cells and the marker gene were defined. Cell deconvolution was attempted on 24 lung cells using an MCP-counter referencing the marker gene, through a mouse gene expression profile. Equivalent estimation was performed on the TCGA lung adenocarcinoma expression profile, and mean cell viability for each isoform defined in the TCGA study was summarized.

8-11. Cell Proliferation

Growth was examined using MEFs derived from 13.5-day-old embryos. Cells were plated on a 6-well plate at a density of 20,000 cells/well, and then the number of the cells was counted every day from the following day for 5 days. To examine cell survival in response to nocodazole, which is a cell division inhibitor, cells were treated with 50 ng/ml or 100 ng/ml of nocodazole for 48 hours and cultured. Subsequently, the cells were treated with trypsin/EDTA and detached, followed by cell counting.

8-12. Live Cell Imaging

Embryonic epithelial cells plated in a lab-tekII chamber the previous day were transfected with a retro virus capable of expressing a H2B-separase sensor. After 3 days, fluorescence images were captured with a total of three z-stacks at intervals of 5 minutes using by using a microscope (Carl Zeiss, Germany) in a humidified incubator at 37° C. and 5% CO₂ inside a video microscope platform. The z-stacks of the captured images were combined together, and then the intensity of fluorescence in chromosomes for each image was measured using the Zen 2 pro blue software.

### Example 9. Generation of RARRES1 Knockout Mice

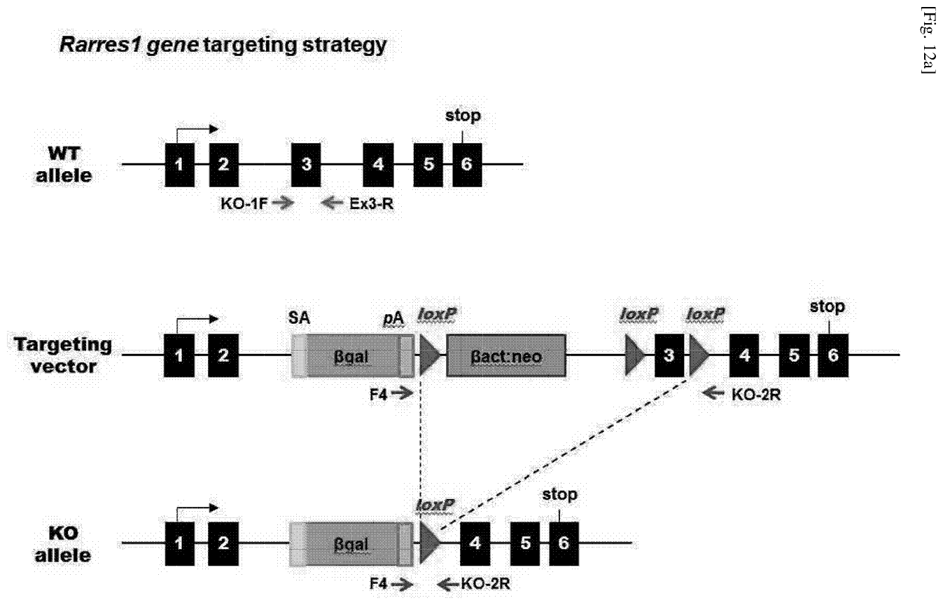

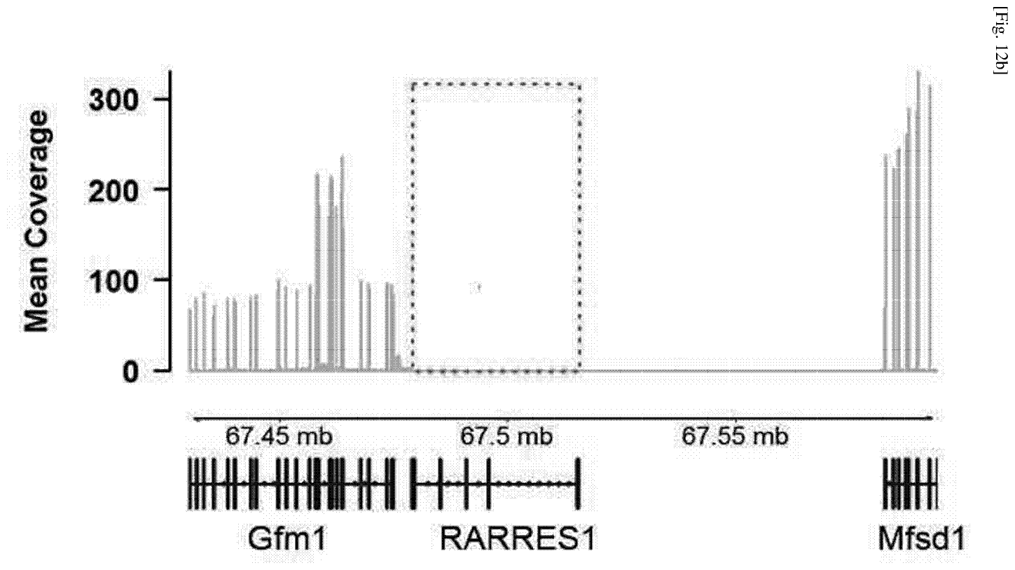

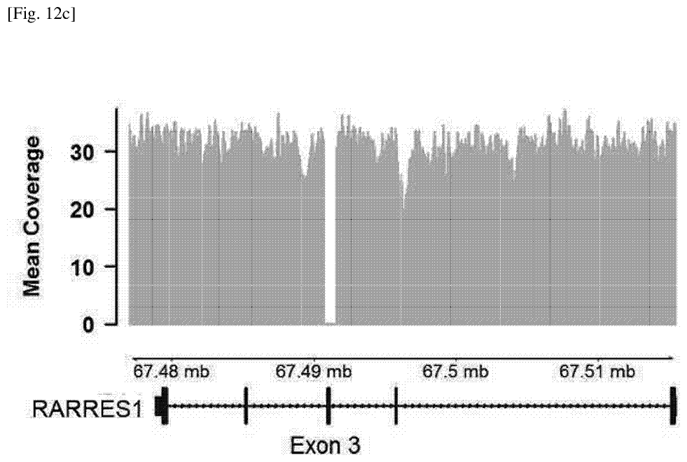

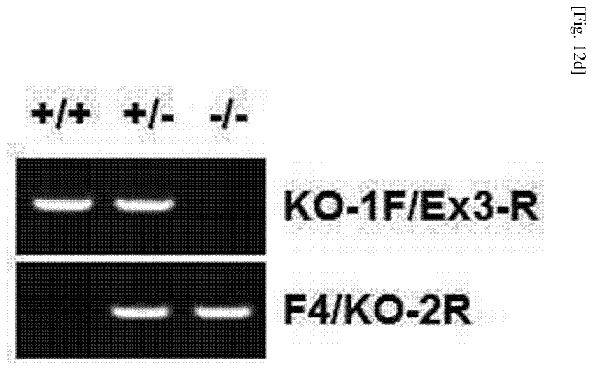

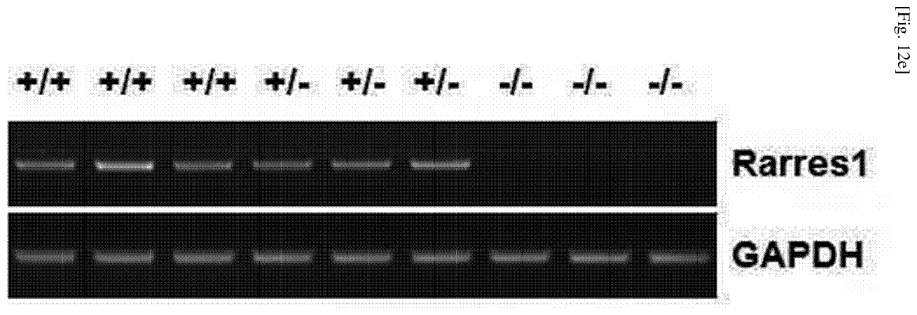

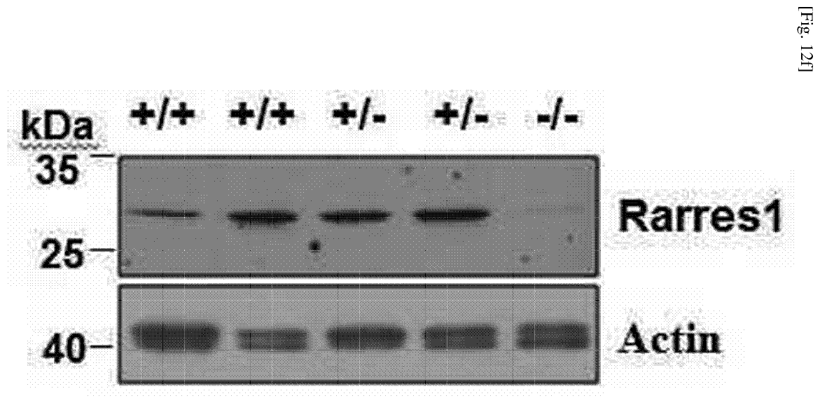

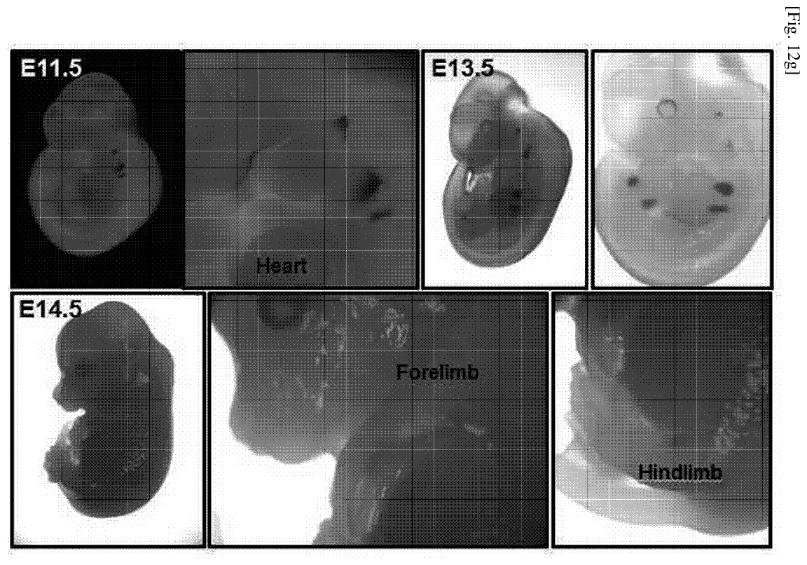

To determine the in vivo physiological function of RARRES1, conditional RARRES1 knockout mice were derived from mouse embryonic stem (ES) cell clones (F07 and A05) for a knockout mouse project (KOMP). A targeting construct containing a splicing acceptor (SA), 3-galactosidase ((3 gal), and an SV40 polyA signal (pA), followed by a floxed neomycin-resistant cassette (neo) controlled by the β-actin promoter, was inserted into intron 2 of the mouse Rarres1 gene. In addition, the third loxP site was inserted into intron 3 and consequently, exon 3 was flanked by two loxP sequences recognized and removed by Cre recombinase (see FIG. 12A). Correctly targeted ES clones were injected into blastocysts to obtain Rarres1^{+/N} progenies. Rarres1^{+/-} mice were established by crossing Rarres1^{+/N} males with transgenic females expressing Cre recombinase in the germ line, and this is controlled by the mouse zona pellucida 3 (Zp3) promoter. In addition, to confirm the production of knockout profiles of Rarres1, the coverage of Rarres1 identified by sequencing of Rarres1 RNA-seq was represented as a table (see FIG. 12B). In addition, it was confirmed through whole genome sequencing that the whole mRNA expression of Rarres1 did not normally occur by deletion of the DNA exon3 sequence (see FIG. 12C). Thus, it was confirmed that the RNA-seq coverage of the corresponding base sequence became 0.

For verification Rarres1^{+/-} mice, which had produced offspring, were intercrossed and verified by PCR genotyping of tail genomic DNA (see FIG. 12D) and RT-PCR (see FIG. 12E) according to the method of Example 1-2 and 8-2 for Rarres1^{+/+}, Rarres1^{+/-}, and Rarres1^{-/-} MEFs. Western blotting analysis of whole embryo lysates on embryonic day 13.5 revealed that while an expression level of the Rarres1 protein was not reduced in Rarres1^{+/-} compared to Rarres1^{+/+}, the level of the Rarres1 protein was low in Rarres1^{-/-} embryos (see FIG. 12F). Due to the fusion between Rarres1 and βgal, the expression of Rarres1 in Rarres1^{+/-} embryos could easily be monitored through LacZ staining according to Example 8-4. As illustrated in FIG. 12G, Rarres1 showed limited expression throughout the whole embryo from E11.5 to E14.5, mainly stained with LacZ according to Example 8-4 around the forelimbs and hindlimbs, and weakly stained in the eyes of the embryo.

In addition, to examine the effect of Rarres1 knockout on embryonic death, intercrossed heterozygotes and genotypes of embryos on day 13.5 and infant mice were surveyed. As shown in Table 1 below, 82 embryos and 165 neonatal mice were present at the expected Mendelian ratio. RARRES1 heterozygous mice and homozygous mice were normal in appearance and health conditions. Thus, this targeted knockout of Rarres1did not affect survival during embryogenesis.

TABLE 1

| Age of | | No. of embryos or infant | | | No. of embryos or infant | | |
|---|---|---|---|---|---|---|---|
| embryo/ | No. of | mice with indicated | | | mice with indicated | | |
| infant | embryos/ | actual genotype | | | expected genotype | | |
| mice | infants | +/+ | +/− | −/− | +/+ | +/− | −/− |
| E13.5 | 82 | 20 | 41 | 22 | 20 | 42 | 20 |
| After | 165 | 48 | 81 | 36 | 41 | 83 | 41 |
| birth | | | | | | | |

Next, it was tested whether Rarres1-deficient mice had infertility by crossing Rarres1^{-/-} females with Rarres1^{+/-} males or crossing Rarres1^{-/-} males with Rarres1^{+/-} females. Regardless of gender, when KO mice have a problem in fertility, offspring cannot be born. Neonatal animals survived as expected according to the Mendelian law of inheritance and seemingly normal and healthy, suggesting that Rarres1-deficient mice have normal fertility (see Table 2).

TABLE 2

| | | No. of embryos or | | No. of embryos or | |
|---|---|---|---|---|---|
| | | infant mice with | | infant mice with | |
| | | indicated actual | | indicated expected | |
| | No. of | genotype | | genotype | |
| [symbol] | infants | +/− | −/− | +/− | −/− |
| −/−Female | 77 | 41 | 36 | 39 | 38 |
| After birth | | | | | |
| −/−Male | 82 | 44 | 38 | 41 | 41 |
| After birth | | | | | |

### Example 10. Identification of Non-Lymphoid Hematopoietic Neoplasia of Rarres1Knockout Mice

To confirm the possibility of non-lymphoid neoplasia in Rarres1 knockout mice (KO mice), bone marrow cells, spleen cells, and peripheral blood cells were extracted from the genotype profiles of Rarres1^{+/+} and Rarres1^{-/-}. The cells were allowed to react with the following antibodies in FACS buffer (1×PBS with 0.1% bovine calf serum and 0.05% sodium azide) at 4° C. for 30 minutes: eFluor 450-conjugated anti-mouse hematopoietic lineage antibody cocktail [CD3 (17A2), CD45R (RA3-6B2), CD11b (M1/70), TER-119 (TER-119), Gr-1 (RB6-8C5)] (eBioscience, San Diego, Calif., USA), eFluor 450-conjugated anti-Ly-6G (RB6-8C5, eBioscience), eFluor 450-conjugated anti-CD3ε(145-2C11, eBioscience), Alexa Fluor 488-conjugated anti-CD8α(53-6.7, eBioscience), phycoerythrin-cyanine7 (PE-Cy7)-conjugated anti-CD11b (M1/70, eBioscience), allophycocyanin-eFluor 780 (APC-eFluor 780)-conjugated anti-CD11c (N418, eBioscience), APC-eFluor 780-conjugated anti-CD4 (GK1.5, eBioscience), Alexa Fluor 488-conjugated anti-Sca-1 (D7, Biolegend, San Diego, Calif., USA), PE-conjugated anti-CD45R (RA3-6B2, BioLegend), PE-conjugated anti-CD25 (PC61, BioLegend), Alexa Fluor 647-conjugated anti-CD117 (2B8, BioLegend), Alexa Fluor 647-conjugated anti-Ly-6c (HK1.4, BioLegend), PE-Cy7 conjugated anti-CD16/32 (93, BioLegend), PE-Cy7 conjugated anti-CD19 (6D5, BioLegend), Brilliant Violet 650 (BV650)-conjugated anti-CD11b (M1/70, BioLegend), BV650-conjugated anti-I-A/I-E (M5/114.15.2, BioLegend), and BV650-conjugated anti-CD44 (IM7, BioLegend).

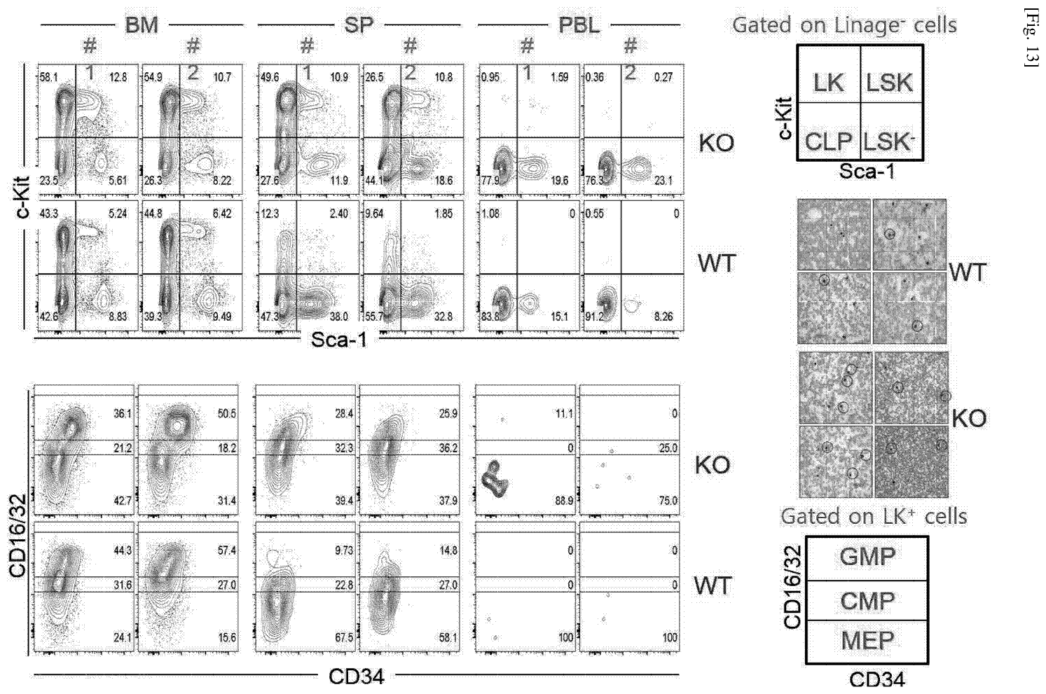

For intracellular staining, the cells were fixed with 4% paraformaldehyde at room temperature for 20 minutes. After fixation, the cells were washed with 1×PBS, cell permeability was secured with 0.5% Triton X-100, and the cells were stained with Alexa Fluor 647-conjugated anti-FOXP3 (MF-14, BioLegend) at room temperature for 1 hour. The intensity of fluorescence of the stained cells was analyzed by BD LSR-Fortessa (BD Bioscience, San Jose, Calif., USA) and the results thereof were analyzed using the FlowJo software (TreeStar, Ashland, Oreg., USA). From these results, it was confirmed that the groups of c-Kit positively stained cells and Sca-1 negatively stained cells, known as myeloid cell progenitors, were increased in the knockout profile group, and common myeloid progenitor (CMP) and granulocyte, monocyte progenitor (GMP) cells were increased in the knockout spleen. An increase in the number of myeloid cells was observed in the actual peripheral blood (see FIG. 13).

### Example 11. RARRES1 Knockout (KO) Mice Prone to Spontaneous Tumors

To confirm spontaneous tumor formation in Rarres1 KO mice, cohorts of Rarres1^{+/+} (n=51), Rarres1^{+/-} (n=47), and Rarres1^{-/-} (n=59) mice were established for intercrossed RARRES1 heterozygous mice (C57BL/6), and observed up to 22 months old. Animals were subjected to necropsy immediately after death during experimental processes, or sacrificed by CO₂ asphyxiation and subjected to necropsy to search for tumors in these mice.

After necropsy, solid organs of each mouse were immediately fixed in 10% neutral buffered formalin, and the fixed tissues were made into paraffin blocks and sections, followed by general hematoxylin/eosin staining for histopathological examination. In histopathologic examination of each organ, abnormal proliferation distinct from normal histologic structures were defined as tumors, neoplasms that had a pattern of pressing surrounding normal tissues and did not exhibit invasive and metastatic behavior were diagnosed as benign, and neoplasms exhibiting invasive or metastatic behavior were diagnosed as malignant.

As a result, it was found that Rarres1^{+/-} and Rarres1^{-/-} mice were more prone to develop spontaneous tumors compared to Rarres1^{+/+} mice. In addition, it was confirmed that the Rarres1^{+/-} and Rarres1^{-/-} mice developed different types of tumors in organs including the spleen, thymus, liver, lungs, kidneys, thyroid, small intestine, stomach, endometrium, and eyes, unlike the Rarres1^{+/+} mice (see Table 3). The Rarres1^{-/-} mice developed malignant tumors in various major organs such as the liver, lung, stomach, and thyroid gland, and thyroid carcinoma metastasized to liver (see Table 3 and FIGS. 14A, 14B, and 14C). In addition, unlike the Rarres1^{+/+} mice, the Rarres1^{-/-} mice developed more advanced forms of T cell lymphomas related to multiple organs such as the thymus, kidneys, bladder, and the like, and this was confirmed through immunohistochemical staining for CD3, which is a T cell marker (see Table 3 and FIG. 14D). In addition, it was confirmed through immunohistochemical staining for myeloperoxidase, which is a myeloid cell marker, that bone marrow- and spleen-associated myeloid leukemia more frequently occurred in the Rarres1^{-/-} mice (see FIG. 14E). Meanwhile, regarding this, the sizes of organs including the spleen, liver, and kidneys were gradually increased in 19-month-old mice according to genotype (see FIG. 15).

In addition, to monitor tumor growth in the groups of Rarres1^{+/+} (n=6), Rarres1^{+/-} (n=6), and Rarres1^{-/-} (n=6) mice through PET/CT according to Example 8-5, the same mice aged between 6 months and 15 months were observed for two months. It was confirmed through [¹⁸F] FDG PET/CT imaging according to Example 8-5 that until 10 months after birth, there was no difference in fludeoxyglucose (FDG) uptake between KO mice and WT mice, but when compared to wild-type mice having an age similar to that of 10-month-old mice, the intensity of FDG uptake was increased mainly around spinal cord of Rarres1-deficient mice. When compared to WT mice, the intensity of FDG uptake was gradually increased in the liver of Rarres1 KO mice at 14.5 months (2/6 (33%)). It was confirmed through the PET/CT imaging according to Example 1-9 that tumors occurred much earlier in the Rarres1 KO mice than in the WT mice (see FIG. 16).

Taken altogether, the causal relationship between knockdown of Rarres1 expression and cancer development was established, and the fact that tumor formation was sufficiently induced only by Rarres1deletion was confirmed.

TABLE 3

| | Wild-Type (n = 51) | | Hetero (n = 47) | | RARRES1 KO (n = 59) | |
|---|---|---|---|---|---|---|
| Epithelial tumor | Adenoma | Carcinoma | Adenoma | Carcinoma | Adenoma | Carcinoma |
| Stomach | 0 | 0 | 0 | 1 | 2 | 1 |
| Small intestine | 1 | 0 | 0 | 0 | 1 | 0 |
| Large intestine | 0 | 0 | 0 | 0 | 1 | 0 |
| Lung | 1 | 0 | 2 | 0 | 2 | 1 |
| Liver | 0 | 0 | 0 | 0 | 1 | 2 |
| #Thyroid gland | 0 | 0 | 0 | 0 | 0 | 1 |
| subtotal | 2 | 0 | 2 | 1 | 7 | 5* |

| | | | |
|---|---|---|---|
| | 2(3.92%) | 3(6.38%) | 12(20.3%)** |
| Lymphoma | | | |
| Focal | 4 | 5 | 15 |
| Multifocal | 2 | 2 | 2 |
| Systemic | 0 | 0 | 2† |
| Subtotal | 6(11.8%) | 7(14.9%) | 19(32.2%)* |
| Histiocytic sarcoma | | | |
| Focal | 5 | 12 | 10 |
| Multifocal | 7 | 7 | 6 |
| Subtotal | 12(27.5%) | 19(40.4%) | 16(27.1%) |
| §Other tumor | 0 | 2 | 2 |
| Total | 19(37.3%) | 25(53.2%) | 37(62.7%)** |

*p value < 0.05 and
**p value < 0.01 versus WT mice
§other tumor: leiomyoma in uterus and leiomyosarcoma in small intestine of RARRES1 KO; hemangiosarcoma in skin and luteoma in ovary of RARRES1 heterotype.
†systemic lymphoma related to parenchymal organ such as liver, kidney and urinary bladder
#Thyroid gland carcinoma with metastasis to liver

### Example 12. Cell Cycle Progression Through Fine Tuning Regulation of CDK1-Cyclin B1 Activity in Rarres1 Knockout (KO) Mice

It was found that RARRES1 suppressed the activity of CDK1-Cyclin B1 in mitosis, and tumorigenesis was increased in Rarres1 KO mice. In addition, it was confirmed that Cyclin B1 transgenic mice were highly prone to tumors. Through these results, to test whether the activation of CDK1-Cyclin B1 regulated by direct binding of RARRES1 to each component promotes tumor formation in Rarres1 KO mice, wild-type and Rarres1-deficient MEFs according to Example 8-3 from embryos on embryonic day 13.5 for in vitro culture was prepared, and western blotting was performed thereon. As expected, phosphorylation at threonine 14 and 161 residues of CDK1 was enhanced in KO MEFs. In addition, Cyclin B1 phosphorylation (serine 126, 128, 133, and 147) was accumulated more in Rarres1-null MEFs compared to that of WT counterparts. CDK activity was measured using an antibody against phosphorylated CDK substrates that detect phospho-serine in a (K/R)(S*)PX(K./R) motif, which is sequence of a CDK substrates, and this was increased in null MEFs. Rb, which is phosphorylated and dephosphorylated in G1 and phosphorylated from the S to M phases of the cell cycle, is a substrate of CDK1, and is phosphorylated by active CDK1-Cyclin B1 complexes in mitosis. The Rb protein was increased and phosphorylated in KO MEFs. Separase, which is another substrate of CDK1, is cleaved in KO cells, which indicates that CDK1-Cyclin B1 activity was exhibited due to the loss of Rarres1 (see FIG. 17).

Next, it was examined whether an increase in CDK1-Cyclin B1 activity can affect cell growth in MEF cells. The MEF cells were seeded in a 6-well plate at a density of 2×10⁴ cells/well three times, and cell counting was performed every day for 5 days. When compared to Rarres1^{+/+} cells, Rarres1^{-/-} MEFs grew more quickly (see FIG. 18A).

To evaluate whether the enhanced tumor proliferation observed in the Rarres1^{-/-} MEFs was due to a changed cell cycle progression, transition from the G1 to G2/M phases and from the G2/M to G1 phases was studied using the cell synchronization experiment according to Example 8-1. Serum starvation (0.1% FBS) for 72 hours followed by serum stimulation (10% FBS) for a maximum of 42 hours indicated that the progression of G1 to G2/M phases of the cell cycle was more rapid in Rarres1^{-/-} cells than in WT cells and the Rarres1^{-/-} cells lasted much longer in the G2/M phases than in the WT cells (see FIG. 18B). The expression of Cyclin D, known to be expressed during the G1 phase and to bind to and activate CDK4/6 during G1 to prepare for DNA synthesis, peaked at 24 hours after release in a fresh medium from serum starvation in WT cells, but this protein expression was downregulated during overall time periods in KO cells. The activation of CDK4/6-Cyclin D complexes inactivates retinoblastoma(Rb) protein during the G1 phase by multi-phosphorylation, which is referred to as “hypo-phosphorylation.” Rb phosphorylation peaked in both cell lines within 27 hours after serum stimulation. However, in cells lacking Rarres1, phosphorylation was maintained up to 42 hours. Rb binds to E2F transcription factors in the early G1 phase, hypo-phosphorylated Rb leads to release of E2F that allows the expression of Cyclin E, which binds to and activates CDK2, resulting in activation of CDK2-Cyclin E complexes that inactivate the Rb protein by hyper-phosphorylation. In WT cells, the expression of Cyclin E peaked at 30 hours after serum stimulation and decreased quickly, but Cyclin E peaked at 30 hours and decreased slightly in KO cells (see FIGS. 18C and 18D). Using a nocodazole-release method coupled with FACS and western blotting analyses, it was confirmed that mitotic exit was fast in RARRES1-null cells compared to WT cells (see FIG. 18E). These differences in the cell cycle were accompanied by a difference in the activation of CDK1 and Cyclin B1. KO cells enhanced the active phosphorylation (threonine 161) of CDK1 and phosphorylation (serine 126, 133, and 147) of Cyclin B1. High CDK1 activity leads to separation of sister chromatids by controlling the activity of separase, but results in incompleted cytoplasmic division. Separase expression was increased during an overall time period and cleaved by its activation after 18 hours, and in KO cells, phosphorylation of Rb, which is a CDK1 substrate, was enhanced up to 18 hours from 6 hours after release (see FIG. 18F), which indicates that Rarres1^{-/-} cells increased CDK1-Cyclin B1 activity. Through these results, it was confirmed that the activity of CDK1-Cyclin B1 complexes was higher in Rarres1 KO MEFs than in WT MEFs, and thus the timing of cell cycle progression was inappropriate and rapid.

### Example 13. Loss of Rarres1 Causing Mitotic Defects and Chromosome Instability

It was examined whether mitotic defects occurred in RARRES1-deficient cells, and through this, the high activity of CDK1-Cyclin B1 complexes and dysregulated cell cycle progression were confirmed. Several types of mitotic errors, including chromosome misalignment, chromatin bridges, and lagging chromosomes, were observed (see FIG. 19A). As a result of immunofluorescence analysis according to Example 8-6, it was confirmed that chromosome misalignment and missegregation occurred in KO cells during the mitosis of Rarres1 KO MEFs (see FIGS. 19B and 19C).

A recent report demonstrates that chromosome segregation errors in mitosis leads to chromosomal abnormalities, including aneuploidy (numerical) and structural abnormalities (translocations and deletions), and thus to confirm whether the knockdown of Rarres1 affects chromosomal stability, metaphase spreads according to Example 8-7 in the Rarres1^{+/+} and Rarres1^{-/-} MEFs according to Example 8-3 cultured with colcemide were analyzed and chromosome numbers were determined. At passage 5 (P5), Rarres1^{+/+} MEFs had almost normal karyotypes, but 20% or more of Rarres1^{-/-} MEFs at metaphase exhibited substantial aneuploidy or polyploidy (see Table 4).

TABLE 4

| | Mitotic | | | | | | | | |
|---|---|---|---|---|---|---|---|---|---|
| MEF | cells | Karyotypes with the indicated chromosome number | | | | | | | |
| genotype(n) | inspected | <38 | 39 | 40 | 41 | 42 | 43 | 70-79 | >80 |
| Rarres1^{+/+}(3) | 100 | 2 | 5 | 87 | 3 | 3 | | | |
| Rarres1^{+/-}(3) | 100 | 3 | 5 | 73 | 10 | 4 | 1 | 1 | 3 |
| Rarres1^{-/-}(3) | 100 | 4 | 7 | 59 | 9 | 1 | 2 | 7 | 11 |

The structural abnormalities of MEF cells were examined. Rarres1-deficient cells exhibited substantial DNA damage both in the micronuclei and the primary nuclei as measured by damage-dependent phosphorylation of the histone variant H2AX (γ-H2AX foci formation). In comparison, γ-H2AX foci were rarely found in WT MEFs (see FIGS. 19C and 19D). Taken altogether, it was confirmed that chromosome missegregation could be increased by the occurrence of DNA damage foci and aneuploidy in RARRES1-deficient cells.

### Example 14. Nocodazole Resistance in Rarres1 Knockout (KO) Cells

It was determined whether a mitotic stress-inducing drug such as nocodazole affects Rarres1 KO MEFs. MEF cells were treated with nocodazole (50 ng/ml or 100 ng/ml) or DMSO for 48 hours, and flow cytometry and cell counting according to Example 1-10 were performed thereon. As a result, cell death induced by nocodazole was significantly reduced in Rarres1-deficient MEFs in a dose-dependent manner (see FIGS. 20A and 20B), and the number of the cells was less reduced by nocodazole treatment than in WT MEFs (see FIG. 20C). Consistent with these results, it was confirmed that the cleavage of PARP and Caspase 3 was decreased in KO cells as compared to WT cells (see FIG. 20D), and the loss of Rarres1 caused resistance to the nocodazole treatment.

### Example 15. Increase in Phosphorylation of CDK1 and Cyclin B1 in Solid Tumors of RARRES1-Deficient Mice

Immunohistochemical (IHC) staining according to Example 8-8 was performed on phospho-Cyclin B1-ser126 and phospho-CDK1-T161 in liver cancer and lung cancer occurring in RARRES1 KO cells. The two antibodies were negatively stained in liver and lung sections of WT mice, but phospho-Cyclin B1-ser126 and phospho-CDK1-T161 were strongly stained in tumor sections of RARRES1-deficient mice, and the two antibodies were negatively stained around cancer cells (see FIG. 21A). These IHC results suggest that the activity of CDK1-Cyclin B is associated with tumorigenesis in RARRES1-deficient mice. Meanwhile, as a result of performing immunohistochemical staining for Ki67, CDK1, and a phosphorylated Rb protein, which are cell cycle activation markers, on mouse lung tissues having the same age (18 months old), a greater number of cells exhibiting positive reactivity to the above three markers were observed in Rarres1^{+/-} and Rarres1^{-/-} mice than that in Rarres1^{+/+} (see FIG. 21B).

### Example 16. Rarres1 Contributing to Organ-Blast Cell Homeostasis

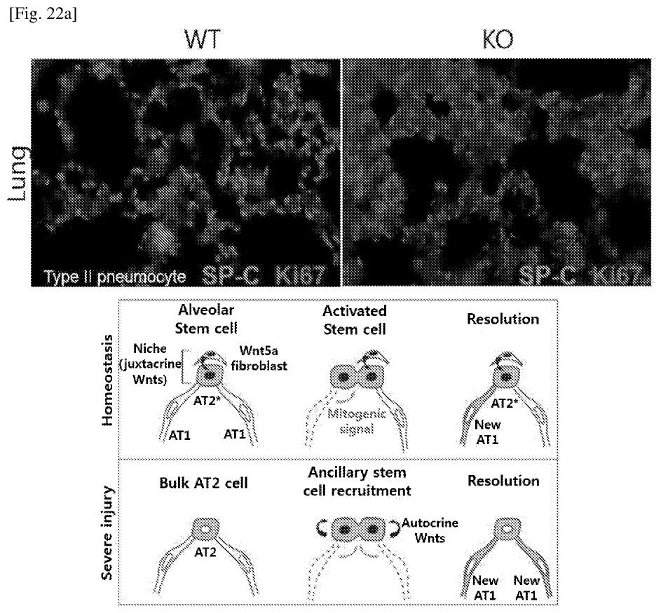

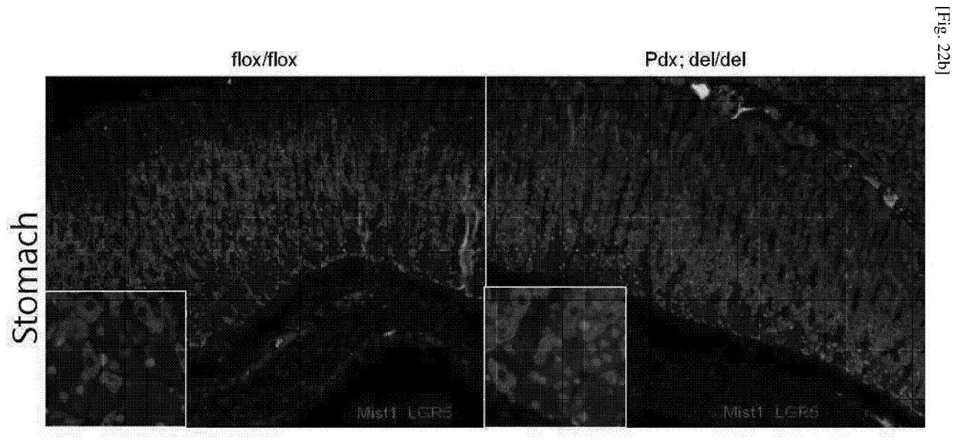

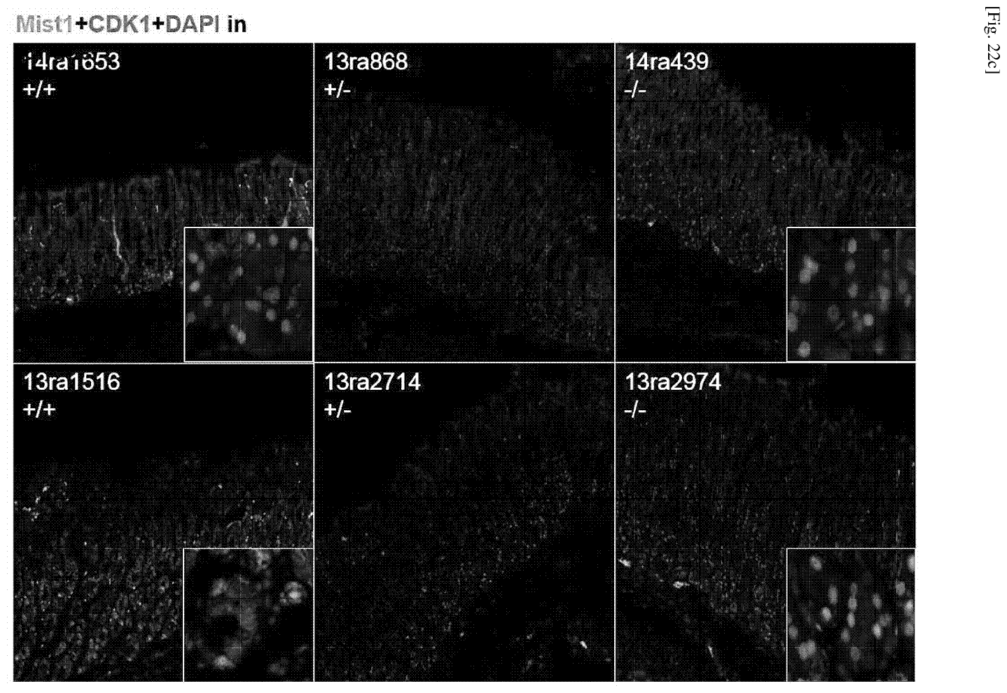

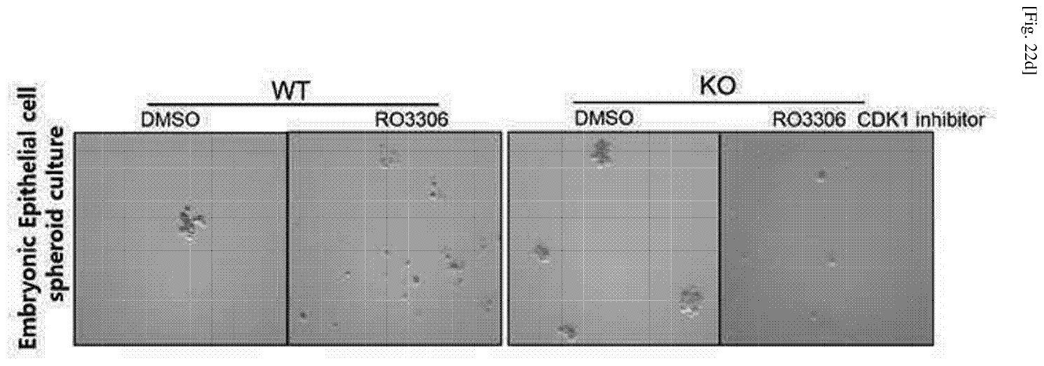

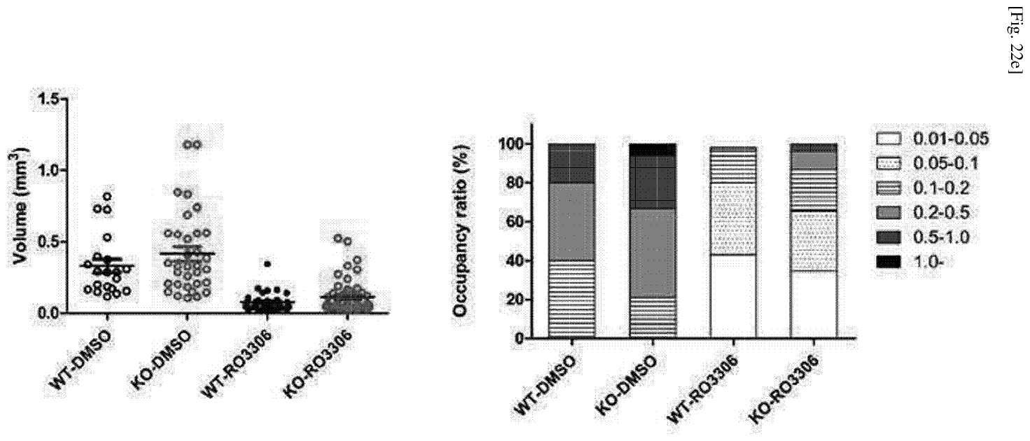

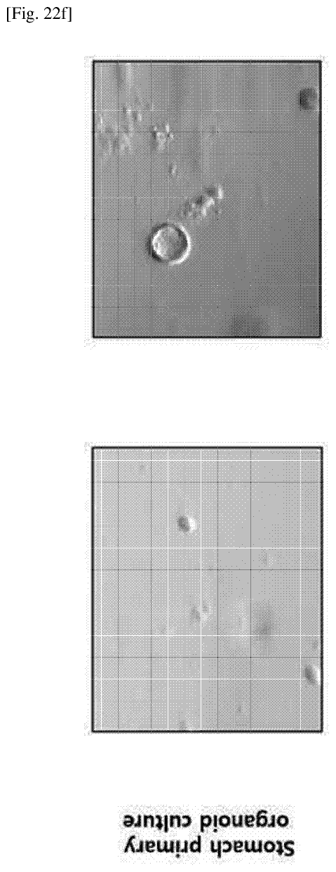

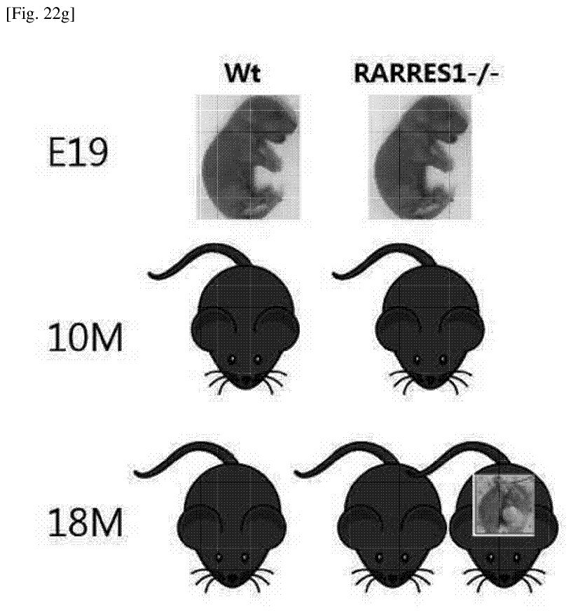

In co-immunofluorescence for surfactant protein C (SPC) marking alveolar type II cell and Ki67 marking cells in active cell cycle, lung from Rarres1^{-/-} mice displayed large numbers of double positive cells compared to Rarres1^{+/+} mice, suggesting that Rarres1 deficiency promotes cell proliferation of alveolar type II cell (see FIG. 22A). As a result of co-immunofluorescence for Mist1 and LGR5 (3 months old stomach-specific Rarres1^{-/-} or Rarres1^{+/+} mice; FIG. 22B) or Mistzx1 and CDK1 (18 months old whole body Rarres1^{-/-} or Rarres1^{+/+} mice; FIG. 22C), which are stomach-specific stem cell markers, on stomach tissues great numbers of stomach-specific stem cells were observed both in the whole body Rarres1-deficient mice and stomach-specific Rarres1deficient mice, as compared to control mice (see FIGS. 22B and 22C).

In spheroid-formation assay, as mentioned above, advanced DMEM/F12 media used for embryonic epithelial cell culture were employed. The cells from the embryo of Rarres1^{+/+} and Rarres1^{-/-} mice were suspended and seeded in 3000 of a medium with or without RO3306, CDK1 inhibitor, in a 24-well plate at a density of 2,000 or 5,000 cells per well. The size and number of spheres formed while adding 300 μl of a medium once three to four days were measured. As a result, in a group not treated with RO3306, the spheroid formation of the embryonic epithelial cells obtained from the Rarres1-deficient mice was more active than that in control mice. Meanwhile, it was confirmed that overall spheroid formation was significantly reduced in the RO3306-treated, which indicates that the activity of CDK1 is a crucial factor in spheroid formation. In addition, it was confirmed that while the embryonic epithelial cells of the control mice barely formed spheres, the embryonic epithelial cells obtained from the Rarres1-deficient mice formed spheres, which indicates that the deficiency of Rarres1 increases the activity of CDK1, thus maintaining stemness (see FIGS. 22D and 22E).

In gastric organoid culture, the stomach was extracted from each mouse, and then the fundus and pylorus of the stomach were separated from each other and separately chopped in an 8 mM EDTA solution, followed by culture at 4° C. for 1 hour. Subsequently, through centrifugation and filtering, the resulting sections were separated into single cells. Thereafter, the single cells were suspended with Matrigel and then seeded in a 48-well plate. The cells were incubated at 37° C. for about 5 minutes to about 10 minutes to harden the Matrigel, and then advanced DMEM/F12 media supplemented with a growth factor were added around the Matrigel. Thereafter, the media and the growth factor were replaced and maintained once two to three days. As a result, it was confirmed that stomach organoids obtained from stomach-specific Rarres1-deficient mice were formed more rapidly than from control mice (see FIG. 22F).

### Example 17. Somatic Cell Modification Analysis Using Whole Genome Sequencing

As shown in Table 5, all non-silent somatic mutations were found. In particular, mutations in BRAF p.V637E of mice coincided with targets of Vemurafenib human BRAF p.V600E variants. This indicates that the Rarres1 KO mouse model induces somatic mutations that can be targeted. Bc191 nonsynonymous (p.S898T) and Gnas (p.N964delinsNG) non-frame shift insertions were found in cancer-related genes. All somatic mutations are shown in Table 6.

TABLE 5

| | | | | | | Gene. | ExonicFunc | AAChange |
|---|---|---|---|---|---|---|---|---|
| Sample | Chr | Start | End | Ref | Alt | refGene | refGene | refGene |
| 6T | chr6 | 39627783 | 39627783 | A | T | Braf | nonsynonymous SNV | exon18: c.T1910A: p.V637E |
| 4T | chr2 | 174345439 | 174345439 | — | CGG | Gnas | nonframeshift insertion | exon8: c.2891_2892insCGG: p.N964delinsNG |
| 5T | chr9 | 44507447 | 44507447 | T | A | Bcl9l | nonsynonymous SNV | exon7: c.T2692A: p.S898T |

TABLE 6

| Cell | Marker |
|---|---|
| Alveolar bipotent | Krt8,Emp2,Aqp5,Sftpd,Sftpa1 |
| progenitor | |
| Alveolar macrophage | Ear2,Ear1,Cd68,Marco,Siglecf,Chil3,Pclaf,Marco,Siglecf, |
| | Ccna2 |
| AT1 Cell | Ager,Igfbp2,Hopx,Clic5,Pdpn |
| AT2 Cell | Sftpc,Sftpa1,Sftpb,Sfta2,Dram1 |
| B Cell | Cd79a,Ms4a1,Cd79b,Ighd,Cd19 |
| Basophil | Ccl4,Ccl3,Il6,Cd69,Cd200r3 |
| Ciliated cell | Ccdc153,Tmem212,1110017D15Rik,Foxj1,Ccdc17 |
| Clara Cell | Scgb1a1,Aldh1a1,Cyp2f2,Scgb3a1,Hp |
| Conventional dendritic cell | Gngt2,Lst1,Plac8,Itgb2,Cd68,Cd209a,Itgax,Cd74,H2-Eb1, |
| | Itgam,Fscn1,Cc122,Nudt17,H2-M2,Syngr2 |
| Dendritic cell | Naaa,Irf8,Cd74,Itgax,Itgae |
| Dividing cells | Cdc20,Ube2c,Stmn1,Pclaf,Tubb5 |
| Dividing dendritic cell | Cd74,H2-Aa,H2-Ab1,Naaa,Ccnb2 |
| Dividing T cells | Thy1,Cd8b1,Cdk1,Cd3g,Cd3d |
| Endothelial cell | Eng,Kdr,Flt1,Cdh5,Pecam1,Car4,Kdr,Flt1,Cdh5,Pecam1, |
| | Vwf,Kdr,Flt1,Cdh5,Pecam1 |
| Eosinophil granulocyte | G0s2,Clec4d,S100a9,S100a8,Cd14 |
| Ig-producing B cell | Jchain,Igha,Igkc,Ighm,Igkv2-109 |
| Interstitial macrophage | C1qc,C1qa,Pf4,Cd74,Adgre1 |
| Monocyte progenitor cell | Elane,Mpo,Ctsg,Prtn3,Ms4a3 |
| Neutrophil granulocyte | Ngp,S100a9,S100a8,Cd177,Ly6g |
| NK Cell | Nkg7,Klra8,Klra4,Klrb1c,Klra13-ps |
| Nuocyte | Cxcr6,Icos,Thy1,S100a4,Il7r |
| Plasmacytoid dendritic cell | Ms4a6c,Plac8,Bst2,Irf7,Irf5 |
| Stromal cell | Dcn,Col3a1,Fgf10,Tcf21,Hoxa5,Inmt,Gsn,Fgf10,Tcf21, |
| | Hoxa5,Acta2,My19,Fgf10,Tcf21,Hoxa5 |
| T Cell | Trbc2,Cd8b1,Cd3d,Cd3g,Thy1 |

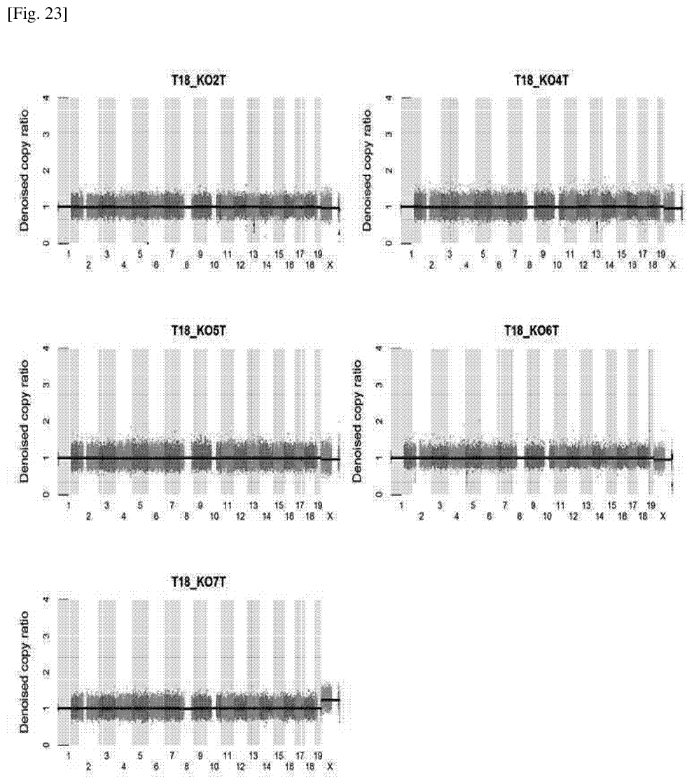

Somatic copy number variants (CNVs) and structural variants (SVs) of all mouse tumor samples (n=5) occurred at a low frequency in consideration of genomic stable cancers. As illustrated in FIG. 23, there were no CNVs exhibiting segmentation-level amplification or deletion in the tumor cells. In the meantime, as illustrated in Table 7, 64 somatic structural variants (SVs) were identified in the tumor samples. Deletions (n=27) and translocations (n=32) occurred most frequently. In particular, in KO4T samples, Cdkn1a deletions are present around chr17 region: 27975841-299991317.

TABLE 7

| SAMPLE | CHROM1 | POS1 | CHROM2 | POS2 | SVCLASS | GENE | CANCERGENE |
|---|---|---|---|---|---|---|---|
| T18_KO2T | chr12 | 111538547 | chr12 | 111539406 | Deletion | Eif5 | . |
| T18_KO2T | chr12 | 111539685 | chr12 | 111539798 | Deletion | Eif5 | . |
| T18_KO2T | chr12 | 111539882 | chr12 | 111540129 | Deletion | Eif5 | . |
| T18_KO2T | chr12 | 111540571 | chr12 | 111541704 | Deletion | Snora28, Eif5 | . |
| T18_KO2T | chr12 | 111541844 | chr12 | 111542150 | Deletion | Eif5 | . |
| T18_KO2T | chr12 | 111542841 | chr12 | 111543099 | Deletion | Eif5 | . |
| T18_KO2T | chr12 | 111543262 | chr12 | 111543524 | Deletion | Eif5 | . |
| T18_KO2T | chr12 | 111543659 | chr12 | 111544526 | Deletion | Eif5 | . |
| T18_KO2T | chr5 | 45985059 | chr5 | 45986594 | Deletion | | . |
| T18_KO2T | chr5 | 121093327 | chr5 | 151725086 | Inversion | | . |
| T18_KO2T | chr7 | 34328736 | chr7 | 34329386 | Deletion | | . |
| T18_KO2T | chr7 | 55643415 | chr12 | 111538242 | Translocation | Eif5 | . |
| T18_KO2T | chr7 | 55643426 | chr12 | 111546484 | Translocation | Eif5 | . |
| T18_KO2T | chr7 | 55643556 | chr12 | 111539298 | Translocation | Eif5 | . |
| T18_KO4T | chr17 | 16978502 | chr17 | 17046447 | Deletion | | . |
| T18_KO4T | chr17 | 27975841 | chr17 | 29991317 | Deletion | Trp53cor1, Mir6969, Stk38, | Cdkn1a, Pim1, |
| | | | | | | Clps, Cdkn1a, Fkbp5, Pim1, | Srsf3, Fance |
| | | | | | | Ppard, Rpl10a, Srsf3, | |
| | | | | | | 4930539E08Rik, Srpk1, Tcp11, | |
| | | | | | | Tead3, Tulp1, Anks1, Zfp523, | |
| | | | | | | Slc26a8, Def6, Cpne5, Fgd2, | |
| | | | | | | Mapk13, Mapk14, Scube3, Brpf3, | |
| | | | | | | Clpsl2, Lhfpl5, Tbc1d22b, | |
| | | | | | | Pnpla1, Rab44, Mtch1, Rnf8, | |
| | | | | | | Kctd20, Armc12, Ccdc167, Ppil1, | |
| | | | | | | 1810013A23Rik, Pxt1, Tmem217, | |
| | | | | | | Fance, Tbc1d22bos, Pi16, Cmtr1, | |
| | | | | | | 1700030A11Rik, Mdga1, BC004004 | |
| T18_KO4T | chr3 | 95073759 | chr10 | 81306111 | Translocation | Pip5k1a, Pip5k1c | . |
| T18_KO4T | chr6 | 90596933 | chr6 | 90638559 | Tandem-duplication | Aldh1l1, Slc41a3 | . |
| T18_KO4T | chr6 | 122323813 | chr6 | 122325457 | Deletion | Phc1 | . |
| T18_KO4T | chr7 | 16475870 | chr7 | 16475933 | Deletion | Npas1 | . |
| T18_KO4T | chr7 | 108528278 | chr5 | 34913634 | Translocation | | . |
| T18_KO4T | chr7 | 119308417 | chr13 | 84011843 | Translocation | | . |
| T18_KO5T | chr11 | 104535053 | chr11 | 104536427 | Deletion | Cdc27 | . |
| T18_KO5T | chr12 | 9077711 | chr12 | 9184781 | Tandem-duplication | | . |
| T18_KO5T | chr12 | 111538547 | chr12 | 111539406 | Deletion | Eif5 | . |
| T18_KO5T | chr12 | 111539882 | chr12 | 111540129 | Deletion | Eif5 | . |
| T18_KO5T | chr12 | 111542841 | chr12 | 111543099 | Deletion | Eif5 | . |
| T18_KO5T | chr12 | 111543659 | chr12 | 111544526 | Deletion | Eif5 | . |
| T18_KO5T | chr14 | 95923489 | chr14 | 95924636 | Deletion | | . |
| T18_KO5T | chr2 | 169539577 | chr2 | 169540156 | Inversion | | . |
| T18_KO5T | chr5 | 75143783 | chr5 | 75145463 | Deletion | Gm19583 | . |
| T18_KO5T | chr6 | 143203342 | chr1 | 40661555 | Translocation | Etnk1 | Etnk1 |
| T18_KO5T | chr7 | 55643330 | chr12 | 111546658 | Translocation | Eif5 | . |
| T18_KO5T | chr7 | 55643415 | chr12 | 111538242 | Translocation | Eif5 | . |
| T18_KO5T | chr7 | 108528280 | chr5 | 34913636 | Translocation | | . |
| T18_KO6T | chr1 | 42137470 | chr1 | 42137678 | Deletion | | . |
| T18_KO6T | chr1 | 127908344 | chr1 | 127908502 | Deletion | Rab3gap1 | . |
| T18_KO6T | chr1 | 171070172 | chr1 | 171077780 | Deletion | | . |
| T18_KO6T | chr15 | 30984772 | chr15 | 30986359 | Tandem-duplication | Ctnnd2 | . |
| T18_KO6T | chr18 | 9575535 | chr14 | 13358092 | Translocation | Synpr | . |
| T18_KO6T | chr19 | 42469585 | chr12 | 97249299 | Translocation | Gm38437 | . |
| T18_KO6T | chr3 | 19700070 | chr11 | 91724710 | Translocation | | . |
| T18_KO6T | chr3 | 109622539 | chr13 | 20487851 | Translocation | Vav3, Elmo1 | Vav3 |
| T18_KO6T | chr3 | 120703425 | chr14 | 70715950 | Translocation | 6530403H02Rik, Xpo7 | Xpo7 |
| T18_KO6T | chr4 | 25544294 | chr13 | 102567888 | Translocation | | . |
| T18_KO6T | chr4 | 125060132 | chr12 | 97601280 | Translocation | Dnali1 | . |
| T18_KO6T | chr5 | 33111812 | chr1 | 126334627 | Translocation | Slc5a1, Nckap5 | . |
| T18_KO6T | chr5 | 81150341 | chr5 | 81150535 | Deletion | Adgrl3 | . |
| T18_KO6T | chr5 | 128099724 | chr1 | 141912421 | Translocation | Tmem132d | . |
| T18_KO6T | chr6 | 90775745 | chr13 | 65106495 | Translocation | Iqsec1, Mfsd14b | . |
| T18_KO6T | chr6 | 94897007 | chr6 | 94897062 | Deletion | | . |
| T18_KO6T | chr7 | 81108995 | chr3 | 62201316 | Translocation | | . |
| T18_KO6T | chr8 | 4479310 | chr7 | 54004741 | Translocation | | . |
| T18_KO6T | chr8 | 57572028 | chr14 | 63280308 | Translocation | Galnt7 | . |
| T18_KO6T | chr9 | 60802071 | chr18 | 35854024 | Translocation | Uaca, Cxxc5 | . |
| T18_KO6T | chrX | 18640983 | chr18 | 33772125 | Translocation | Gm14345, Gm14346, | . |
| | | | | | | Gm10921 | |
| T18_KO6T | chrX | 75637613 | chr9 | 52949211 | Translocation | | . |
| T18_KO7T | chr17 | 50143150 | chr14 | 12743313 | Translocation | Rftn1, Cadps | . |
| T18_KO7T | chr18 | 25838191 | chr12 | 117721570 | Translocation | Rapgef5 | . |
| T18_KO7T | chr18 | 25838370 | chr12 | 117721238 | Translocation | Rapgef5 | . |
| T18_KO7T | chr19 | 17533464 | chr16 | 27971791 | Translocation | Pcsk5 | Pcsk5 |
| T18_KO7T | chr4 | 154211152 | chr15 | 97566353 | Translocation | Megf6 | . |
| T18_KO7T | chr4 | 154211357 | chr15 | 97566889 | Translocation | Megf6 | . |
| T18_KO7T | chr7 | 97887440 | chr7 | 97887904 | Deletion | Pak1 | Pak1 |

| SAMPLE | INFO | FORMAT | TUMOR |
|---|---|---|---|
| T18_KO2T | END = 111539406; SVTYPE = DEL; SVLEN = −859; CIGAR = 1M859D; | PR:SR | 42.15:42.19 |
| | CIPOS = 0, 1; HOMLEN = 1; HOMSEQ = G; SOMATIC; | | |
| | SOMATICSCORE = 115 | | |
| T18_KO2T | END = 111539798; SVTYPE = DEL; SVLEN = −113; CIGAR = 1M113D; | PR:SR | 10.0:51.20 |
| | CIPOS = 0, 4; HOMLEN = 4; HOMSEQ = AGGT; SOMATIC; | | |
| | SOMATICSCORE = 71 | | |
| T18_KO2T | END = 111540129; SVTYPE = DEL; SVLEN = −247; CIGAR = 1M247D; | PR:SR | 46.12:44.25 |
| | SOMATIC; SOMATICSCORE = 87 | | |
| T18_KO2T | END = 111541704; SVTYPE = DEL; SVLEN = −1133; CIPOS = 0, 1; | PR:SR | 54.4:56.9 |
| | CIEND = 0, 1; HOMLEN = 1; HOMSEQ = G; SOMATIC; | | |
| | SOMATICSCORE = 49 | | |
| T18_KO2T | END = 111542150; SVTYPE = DEL; SVLEN = −306; CIGAR = 1M306D; | PR:SR | 41.1:39.19 |
| | CIPOS = 0, 2; HOMLEN = 2; HOMSEQ = GG; SOMATIC; | | |
| | SOMATICSCORE = 85 | | |
| T18_KO2T | END = 111543099; SVTYPE = DEL; SVLEN = −258; CIGAR = 1M258D; | PR:SR | 52.3:62.18 |
| | SOMATIC; SOMATICSCORE = 65 | | |
| T18_KO2T | END = 111543524; SVTYPE = DEL; SVLEN = −262; CIGAR = 1M262D; | PR:SR | 48.0:57.18 |
| | CIPOS = 0, 3; HOMLEN = 3; HOMSEQ = AGG; SOMATIC; | | |
| | SOMATICSCORE = 65 | | |
| T18_KO2T | END = 111544526; SVTYPE = DEL; SVLEN = −867; CIGAR = 1M867D; | PR:SR | 59.4:53.27 |
| | CIPOS = 0, 4; HOMLEN = 4; HOMSEQ = AGGT; SOMATIC; | | |
| | SOMATICSCORE = 96 | | |
| T18_KO2T | END = 45986594; SVTYPE = DEL; SVLEN = −1535; CIPOS = 0, 8; | PR:SR | 27.20:20.15 |
| | CIEND = 0, 8; HOMLEN = 8; HOMSEQ = AGGAAGCT; SOMATIC; | | |
| | SOMATICSCORE = 115 | | |
| T18_KO2T | END = 151725086; SVTYPE = INV; SVLEN = 30631759; INV3; SOMATIC; | PR:SR | 51.2:50.2 |
| | SOMATICSCORE = 33 | | |
| T18_KO2T | END = 34329386; SVTYPE = DEL; SVLEN = −650; CIGAR = 1M650D; | PR:SR | 23.0:21.9 |
| | CIPOS = 0, 40; HOMLEN = 40; HOMSEQ = | | |
| | TCTCTTCTCTTCTCTTCTCTTCTCTTCTCTTCTCTTCTCT; | | |
| | SOMATIC; SOMATICSCORE = 39 | | |
| T18_KO2T | SVTYPE = BND; MATEID = MantaBND: 7051:10:11:0:0:0:1; SOMATIC; | PR:SR | 18.20:26.30 |
| | SOMATICSCORE = 223; BND_DEPTH = 41; MATE_BND_DEPTH = 46 | | |
| T18_KO2T | SVTYPE = BND; MATEID = MantaBND: 7051:9:11:0:0:0:0; IMPRECISE; | PR | 39.19 |
| | CIPOS = −448, 448; SOMATIC; SOMATICSCORE = 101; BND_DEPTH = | | |
| | 41; MATE_BND_DEPTH = 38 | | |
| T18_KO2T | SVTYPE = BND; MATEID = MantaBND: 7051:1:11:0:0:0:1; IMPRECISE; | PR | 44.6 |
| | CIPOS = −285, 286; SOMATIC; SOMATICSCORE = 51; BND_DEPTH = | | |
| | 45; MATE_BND_DEPTH = 44 | | |
| T18_KO4T | END = 17046447; SVTYPE = DEL; SVLEN = −67945; IMPRECISE; | PR | 32.7 |
| | CIPOS = −273, 273; CIEND = −319, 319; SOMATIC; | | |
| | SOMATICSCORE = 61 | | |
| T18_KO4T | END = 29991317; SVTYPE = DEL; SVLEN = −2015476; CIPOS = 0, 5; | PR:SR | 58.2:43.2 |
| | CIEND = 0, 5; HOMLEN = 5; HOMSEQ = TCCCA; SOMATIC; | | |
| | SOMATICSCORE = 21 | | |
| T18_KO4T | SVTYPE = BND; MATEID = MantaBND: 1714:0:1:0:0:0:1; IMPRECISE; | PR | 58.6 |
| | CIPOS = −242, 242; SOMATIC; SOMATICSCORE = 18; BND_DEPTH = | | |
| | 39; MATE_BND_DEPTH = 38 | | |
| T18_KO4T | END = 90638559; SVTYPE = DUP; SVLEN = 41626; CIPOS = 0, 1; | PR:SR | 50.13:50.12 |
| | CIEND = 0, 1; HOMLEN = 1; HOMSEQ = C; SOMATIC; | | |
| | SOMATICSCORE = 95 | | |
| T18_KO4T | END = 122325457; SVTYPE = DEL; SVLEN = −1644; CIPOS = 0, 2; | PR:SR | 42.2:39.8 |
| | CIEND = 0, 2; HOMLEN = 2; HOMSEQ = CT; SOMATIC; | | |
| | SOMATICSCORE = 36 | | |
| T18_KO4T | END = 16475933; SVTYPE = DEL; SVLEN = −63; CIGAR = 1M63D; | PR:SR | 1.0:15.13 |
| | CIPOS = 0, 8; HOMLEN = 8; HOMSEQ = GCCCGCGC; SOMATIC; | | |
| | SOMATICSCORE = 74 | | |
| T18_KO4T | SVTYPE = BND; MATEID = MantaBND: 31880:0:3:0:0:0:0; IMPRECISE; | PR | 37.7 |
| | CIPOS = −261, 261; SOMATIC; SOMATICSCORE = 58; BND_DEPTH = | | |
| | 50; MATE_BND_DEPTH = 40 | | |
| T18_KO4T | SVTYPE = BND; MATEID = MantaBND: 9143:0:1:0:0:0:1; IMPRECISE; | PR | 62.6 |
| | CIPOS = −281, 281; SOMATIC; SOMATICSCORE = 30; BND_DEPTH = | | |
| | 39; MATE_BND_DEPTH = 19 | | |
| T18_KO5T | END = 104536427; SVTYPE = DEL; SVLEN = −1374; IMPRECISE; | PR | 45.4 |
| | CIPOS = −186, 186; CIEND = −185, 186; SOMATIC; | | |
| | SOMATICSCORE = 11 | | |
| T18_KO5T | END = 9184781; SVTYPE = DUP; SVLEN = 107070; CIPOS = 0, 2; | PR:SR | 49.11:42.9 |
| | CIEND = 0, 2; HOMLEN = 2; HOMSEQ = GC; SOMATIC; | | |
| | SOMATICSCORE = 92 | | |
| T18_KO5T | END = 111539406; SVTYPE = DEL; SVLEN = −859; CIGAR = 1M859D; | PR:SR | 38.7:25.5 |
| | CIPOS = 0, 1; HOMLEN = 1; HOMSEQ = G; SOMATIC; | | |
| | SOMATICSCORE = 55 | | |
| T18_KO5T | END = 111540129; SVTYPE = DEL; SVLEN = −247; CIGAR = 1M247D; | PR:SR | 34.4:39.4 |
| | SOMATIC; SOMATICSCORE = 31 | | |
| T18_KO5T | END = 111543099; SVTYPE = DEL; SVLEN = −258; CIGAR = 1M258D; | PR:SR | 34.0:32.6 |
| | SOMATIC; SOMATICSCORE = 45 | | |
| T18_KO5T | END = 111544526; SVTYPE = DEL; SVLEN = −867; CIGAR = 1M867D; | PR:SR | 44.3:36.8 |
| | CIPOS = 0, 4; HOMLEN = 4; HOMSEQ = AGGT; SOMATIC; | | |
| | SOMATICSCORE = 61 | | |
| T18_KO5T | END = 95924636; SVTYPE = DEL; SVLEN = −1147; CIPOS = 0, 2; | PR:SR | 19.2:11.5 |
| | CIEND = 0, 2; HOMLEN = 2; HOMSEQ = TT; SOMATIC; | | |
| | SOMATICSCORE = 46 | | |
| T18_KO5T | END = 169540156; SVTYPE = INV; SVLEN = 579; CIPOS = 0, 11; | PR:SR | 44.2:33.2 |
| | CIEND = −11, 0; HOMLEN = 11; HOMSEQ = ACACACACACC; INV5; | | |
| | SOMATIC; SOMATICSCORE = 16 | | |
| T18_KO5T | END = 75145463; SVTYPE = DEL; SVLEN = −1680; CIPOS = 0, 2; | PR:SR | 11.14:8.12 |
| | CIEND = 0, 2; HOMLEN = 2; HOMSEQ = TA; SOMATIC; | | |
| | SOMATICSCORE = 172 | | |
| T18_KO5T | SVTYPE = BND; MATEID = MantaBND: 20826:0:1:0:0:0:0; SOMATIC; | PR:SR | 49.2:43.2 |
| | SOMATICSCORE = 28; BND_DEPTH = 34; MATE_BND_DEPTH = 43 | | |
| T18_KO5T | SVTYPE = BND; MATEID = MantaBND: 7387:2:3:0:0:0:0; IMPRECISE; | PR | 38.17 |
| | CIPOS = −352, 353; SOMATIC; SOMATICSCORE = 84; BND_DEPTH = | | |
| | 44; MATE_BND_DEPTH = 36 | | |
| T18_KO5T | SVTYPE = BND; MATEID = MantaBND: 7387:0:2:0:0:0:0; SOMATIC; | PR:SR | 23.11:21.8 |
| | SOMATICSCORE = 108; BND_DEPTH = 41; MATE_BND_DEPTH = 46 | | |
| T18_KO5T | SVTYPE = BND; MATEID = MantaBND: 32855:0:2:0:0:0:0; IMPRECISE; | PR | 45.5 |
| | CIPOS = −258, 259; SOMATIC; SOMATICSCORE = 20; BND_DEPTH = | | |
| | 50; MATE_BND_DEPTH = 40 | | |
| T18_KO6T | END = 42137678; SVTYPE = DEL; SVLEN = −208; CIGAR = 1M208D; | PR:SR | 18.3:27.7 |
| | CIPOS = 0, 30; HOMLEN = 30; HOMSEQ = | | |
| | CAGCAGAGTCTTGCCCAACACCCGCAAGGG; | | |
| | SOMATIC; SOMATICSCORE = 35 | | |
| T18_KO6T | END = 127908502; SVTYPE = DEL; SVLEN = −158; CIGAR = 1M158D; | PR:SR | 17.0:33.5 |
| | CIPOS = 0, 9; HOMLEN = 9; HOMSEQ = CACACACAC; SOMATIC; | | |
| | SOMATICSCORE = 13 | | |
| T18_KO6T | END = 171077780; SVTYPE = DEL; SVLEN = −7608; IMPRECISE; | PR | 247.11 |
| | CIPOS = −537, 537; CIEND = −332, 333; SOMATIC; SOMATICSCORE = 19 | | |
| T18_KO6T | END = 30986359; SVTYPE = DUP; SVLEN = 1587; IMPRECISE; | PR | 71.5 |
| | CIPOS = −266, 266; CIEND = −384, 385; SOMATIC; SOMATICSCORE = 10 | | |
| T18_KO6T | SVTYPE = BND; MATEID = MantaBND: 11510:0:1:0:0:0:0; SOMATIC; | PR:SR | 92.2:83.2 |
| | SOMATICSCORE = 32; BND_DEPTH = 49; MATE_BND_DEPTH = 44 | | |
| T18_KO6T | SVTYPE = BND; MATEID = MantaBND: 7967:0:2:0:0:0:1; IMPRECISE; | PR | 84.7 |
| | CIPOS = −281, 281; SOMATIC; SOMATICSCORE = 20; BND_DEPTH = | | |
| | 44; MATE_BND_DEPTH = 66 | | |
| T18_KO6T | SVTYPE = BND; MATEID = MantaBND: 5232:0:1:0:0:0:1; IMPRECISE; | PR | 81.10 |
| | CIPOS = −257, 258; SOMATIC; SOMATICSCORE = 21; BND_DEPTH = | | |
| | 50; MATE_BND_DEPTH = 34 | | |
| T18_KO6T | SVTYPE = BND; MATEID = MantaBND: 8855:0:6:0:0:0:1; IMPRECISE; | PR | 48.6 |
| | CIPOS = −258, 258; SOMATIC; SOMATICSCORE = 10; BND_DEPTH = | | |
| | 49; MATE_BND_DEPTH = 38 | | |
| T18_KO6T | SVTYPE = BND; MATEID = MantaBND: 12704:4:8:0:0:0:0; IMPRECISE; | PR | 85.5 |
| | CIPOS = −236, 237; SOMATIC; SOMATICSCORE = 10; BND_DEPTH = | | |
| | 49; MATE_BND_DEPTH = 41 | | |
| T18_KO6T | SVTYPE = BND; MATEID = MantaBND: 10932:0:1:0:0:0:0; SOMATIC; | PR:SR | 71.2:77.2 |
| | SOMATICSCORE = 25; BND_DEPTH = 56; MATE_BND_DEPTH = 44 | | |
| T18_KO6T | SVTYPE = BND; MATEID = MantaBND: 7966:0:1:0:0:0:0; CIPOS = 0, 1; | PR:SR | 55.2:52.2 |
| | HOMLEN = 1; HOMSEQ = G; SOMATIC; SOMATICSCORE = 16; | | |
| | BND_DEPTH = 44; MATE_BND_DEPTH = 52 | | |
| T18_KO6T | SVTYPE = BND; MATEID = MantaBND: 25541:0:2:0:0:0:1; IMPRECISE; | PR | 84.5 |
| | CIPOS = −250, 251; SOMATIC; SOMATICSCORE = 10; BND_DEPTH = | | |
| | 35; MATE_BND_DEPTH = 48 | | |
| T18_KO6T | END = 81150535; SVTYPE = DEL; SVLEN = −194; CIGAR = 1M194D; | PR:SR | 27.2:34.4 |
| | CIPOS = 0, 8; HOMLEN = 8; HOMSEQ = TGTGTGTG; SOMATIC; | | |
| | SOMATICSCORE = 10 | | |
| T18_KO6T | SVTYPE = BND; MATEID = MantaBND: 9003:0:1:0:0:0:0; SOMATIC; | PR:SR | 83.2:64.2 |
| | SOMATICSCORE = 11; BND_DEPTH = 54; MATE_BND_DEPTH = 42 | | |
| T18_KO6T | SVTYPE = BND; MATEID = MantaBND: 9855:0:2:0:0:0:0; IMPRECISE; | PR | 76.6 |
| | CIPOS = −229, 230; SOMATIC; SOMATICSCORE = 38; BND_DEPTH = | | |
| | 66; MATE_BND_DEPTH = 45 | | |
| T18_KO6T | END = 94897062; SVTYPE = DEL; SVLEN = −55; CIGAR = 1M1I55D; | PR:SR | 2.0:18.8 |
| | SOMATIC; SOMATICSCORE = 13 | | |
| T18_KO6T | SVTYPE = BND; MATEID = MantaBND: 1:7082:12215:0:0:0:1; IMPRECISE; | PR | 49.6 |
| | CIPOS = −271, 272; SOMATIC; SOMATICSCORE = 23; BND_DEPTH = | | |
| | 50; MATE_BND_DEPTH = 70 | | |
| T18_KO6T | SVTYPE = BND; MATEID = MantaBND: 8256:9:10:0:0:0:0; IMPRECISE; | PR | 69.5 |
| | CIPOS = −253, 253; SOMATIC; SOMATICSCORE = 11; BND_DEPTH = | | |
| | 41; MATE_BND_DEPTH = 53 | | |
| T18_KO6T | SVTYPE = BND; MATEID = MantaBND: 1:8069:8070:0:1:0:1; SOMATIC; | PR:SR | 43.6:47.6 |
| | SOMATICSCORE = 50; BND_DEPTH = 47; MATE_BND_DEPTH = 84 | | |
| T18_KO6T | SVTYPE = BND; MATEID = MantaBND: 8011:3:6:0:0:0:1; IMPRECISE; | PR | 86.10 |
| | CIPOS = −244, 244; SOMATIC; SOMATICSCORE = 26; BND_DEPTH = | | |
| | 39; MATE_BND_DEPTH = 44 | | |
| T18_KO6T | SVTYPE = BND; MATEID = MantaBND: 1:41100:41101:0:0:0:0; IMPRECISE; | PR | 65.6 |
| | CIPOS = −270, 271; SOMATIC; SOMATICSCORE = 14; BND_DEPTH = | | |
| | 29; MATE_BND_DEPTH = 44 | | |
| T18_KO6T | SVTYPE = BND; MATEID = MantaBND: 23533:1:2:0:0:0:1; IMPRECISE; | PR | 57.8 |
| | CIPOS = −323, 324; SOMATIC; SOMATICSCORE = 20; BND_DEPTH = | | |
| | 28; MATE_BND_DEPTH = 60 | | |
| T18_KO7T | SVTYPE = BND; MATEID = MantaBND: 13894:0:1:0:0:0:1; CIPOS = 0, 1; | PR:SR | 70.2:61.2 |
| | HOMLEN = 1; HOMSEQ = G; SOMATIC; SOMATICSCORE = 29; | | |
| | BND_DEPTH = 55; MATE_BND_DEPTH = 48 | | |
| T18_KO7T | SVTYPE = BND; MATEID = MantaBND: 10175:2:5:0:0:0:1; IMPRECISE; | PR | 54.5 |
| | CIPOS = −249, 249; EVENT = MantaBND: 10175:2:5:0:0:0:0; | | |
| | SOMATIC; SOMATICSCORE = 0; JUNCTION_SOMATICSCORE = 10; | | |
| | BND_DEPTH = 55; MATE_BND_DEPTH = 39 | | |
| T18_KO7T | SVTYPE = BND; MATEID = MantaBND: 10175:2:5:1:0:0:1; CIPOS = 0, 1; | PR:SR | 47.11:35.10 |
| | HOMLEN = 1; HOMSEQ = G; SVINSLEN = 85; SVINSSEQ = | | |
| | TGAATACTCACCACAGAAGAAGAATAAAGCCCTTTTCCACCAATT | | |
| | CAGTCTTAAGGAGAACTGGCTCCAGCACAGAGGAACTGTG; | | |
| | EVENT = MantaBND: 10175:2:5:0:0:0:0; SOMATIC; SOMATICSCORE = 0; | | |
| | JUNCTION_SOMATICSCORE = 0; BND_DEPTH = 50; | | |
| | MATE_BND_DEPTH = 69 | | |
| T18_KO7T | SVTYPE = BND; MATEID = MantaBND: 19884:0:1:0:0:0:1; CIPOS = 0, 1; | PR:SR | 77.2:76.2 |
| | HOMLEN = 1; HOMSEQ = T; SOMATIC; SOMATICSCORE = 25; | | |
| | BND_DEPTH = 50; MATE_BND_DEPTH = 62 | | |
| T18_KO7T | SVTYPE = BND; MATEID = MantaBND: 2:9296:10229:1:0:0:0; IMPRECISE; | PR | 73.2 |
| | CIPOS = −252, 253; EVENT = MantaBND: 2:9296:10229:0:0:0:0; SOMATIC; | | |
| | SOMATICSCORE = 24; JUNCTION_SOMATICSCORE = 0; BND_DEPTH = | | |
| | 31; MATE_BND_DEPTH = 47 | | |
| T18_KO7T | SVTYPE = BND; MATEID = MantaBND: 2:9296:10229:0:0:0:1; IMPRECISE; | PR | 39.4 |
| | CIPOS = −391, 391; EVENT = MantaBND: 2:9296:10229:0:0:0:0; SOMATIC; | | |
| | SOMATICSCORE = 24; JUNCTION_SOMATICSCORE = 2; BND_DEPTH = | | |
| | 69; MATE_BND_DEPTH = 41 | | |
| T18_KO7T | END = 97887904; SVTYPE = DEL; SVLEN = −464; CIGAR = 1M464D; | PR:SR | 64.0:51.6 |
| | CIPOS = 0, 7; HOMLEN = 7; HOMSEQ = CTGGCCT; SOMATIC; | | |
| | SOMATICSCORE = 16 | | |

### Example 18. Gene Expression Profile and Function Examination

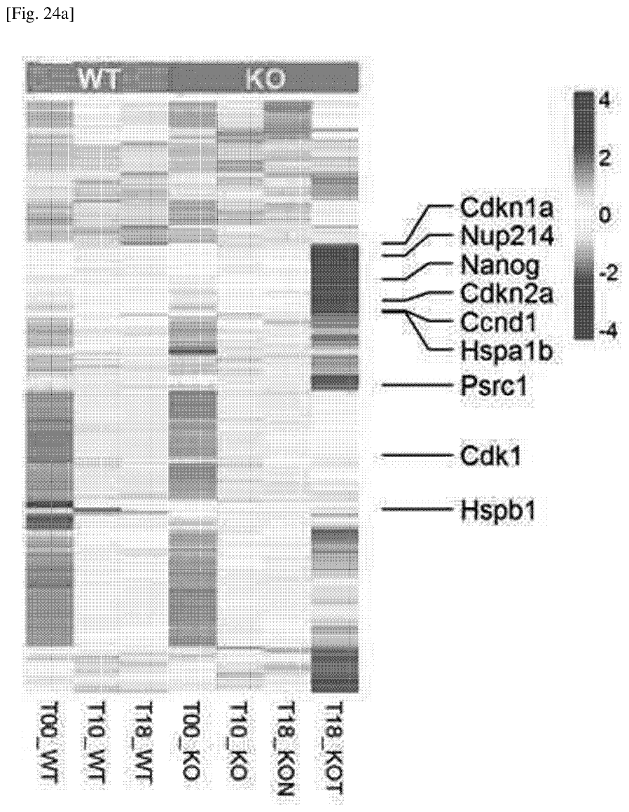

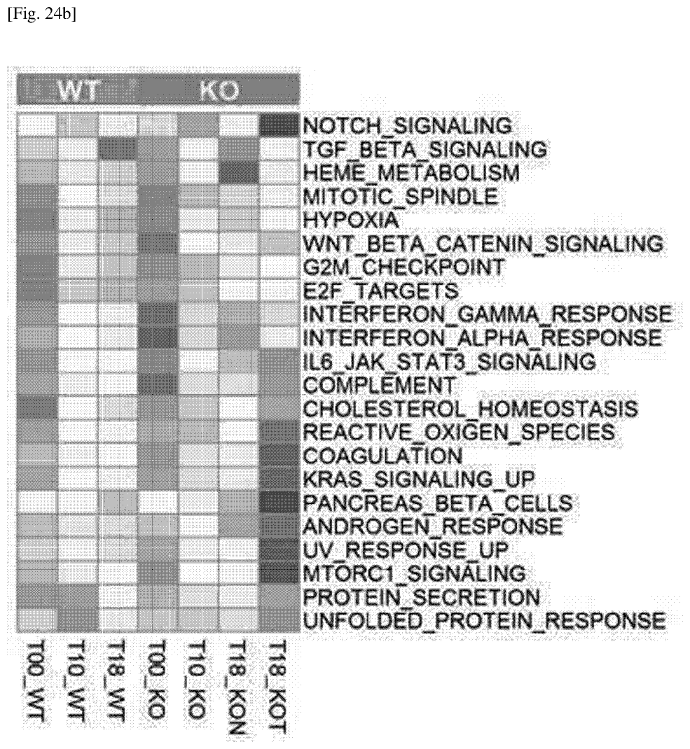

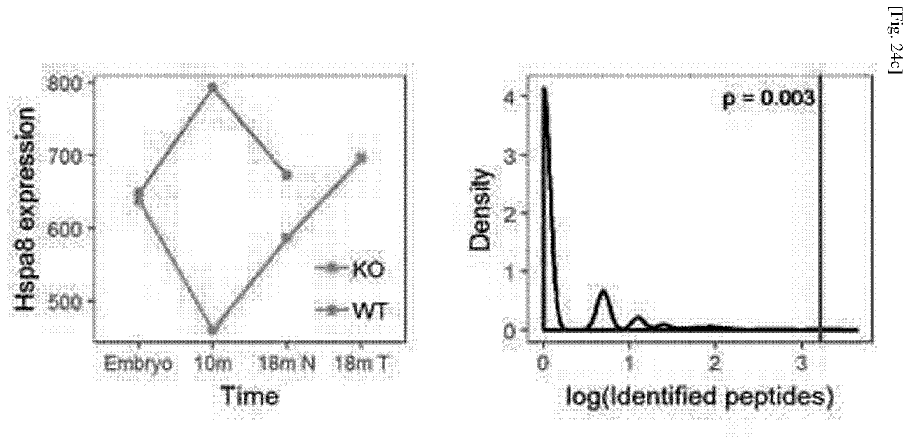

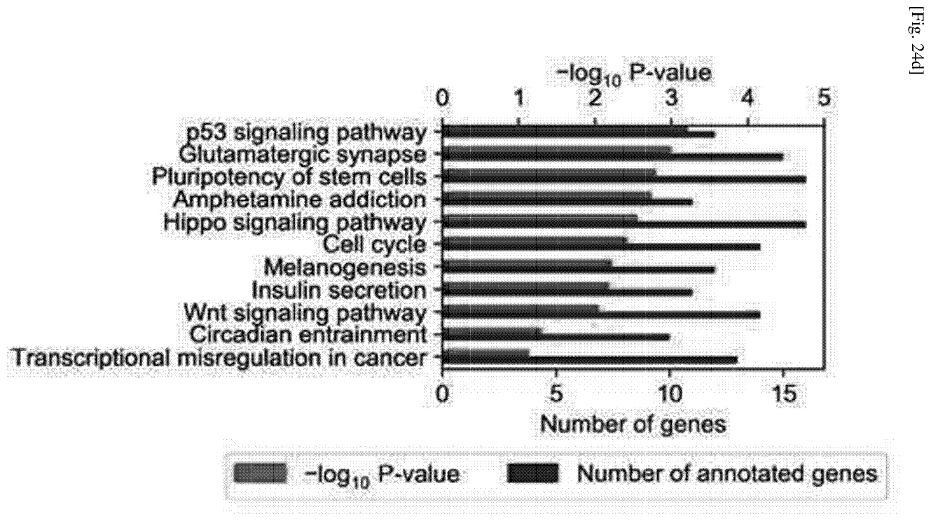

Through gene expression profile analysis, coincident characteristics in each of a plurality of cases were shown. When comparing tumor samples of KO tumor mice with normal samples (FDR<1.0e^{-0.5}), differentially expressed genes were selected depending on the time factor (FDR<1.0e^{-0.4}) described in the method after gene removal. In addition, subnetwork genes were selected and 1,720 genes were selected as a DEG set. As a result of gene set enrichment analysis (GSEA), pathways involved in standard Wnt signaling, cell cycle, and mitosis (see FIG. 24A) were identified, and an additionally unfolded protein response (UPR) was shown to act as an inverse pathway between KO and WT. In contrast, UPR genes, i.e., Ciar, Eif2s1, Hspa5, Hspa8, and Hsp90b1, were downregulated in KO normal compared to WT, but highly expressed in KO tumors (see FIG. 24B). In addition, as illustrated in FIG. 24C, Hspa8 was identified as a binding protein to RARRES1 from IgG evidence. In the Wnt signaling or mitotic cell cycle, Ccnd1, Cdkn1a, Cdkn2A, Nanog, Psrc1, and Nup214 were highly expressed in the KO mouse tumor samples (see FIG. 24D).

CDK1 mRNA exhibited a fold change XXX between KO tumor and KO normal. However, from the comparison between TCGA LUAD RNA-Seq and RPPA, it was confirmed that the correlation between mRNA expression and protein content was low. In addition, the possibility of strong binding between CDK1 and RARRES1 (see FIG. 24C) was verified through an IgG experiment.

### Example 19. RARRES1 State in Deconvolution of TCGA Lung Adenocarcinoma and Lung Cell Content

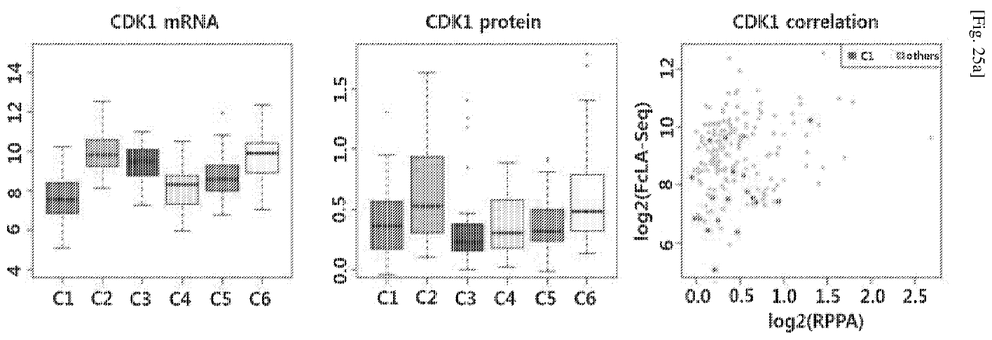

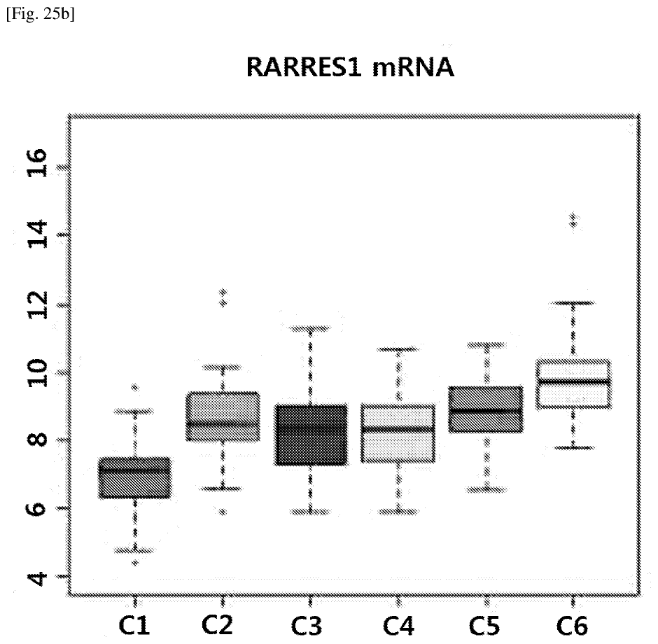

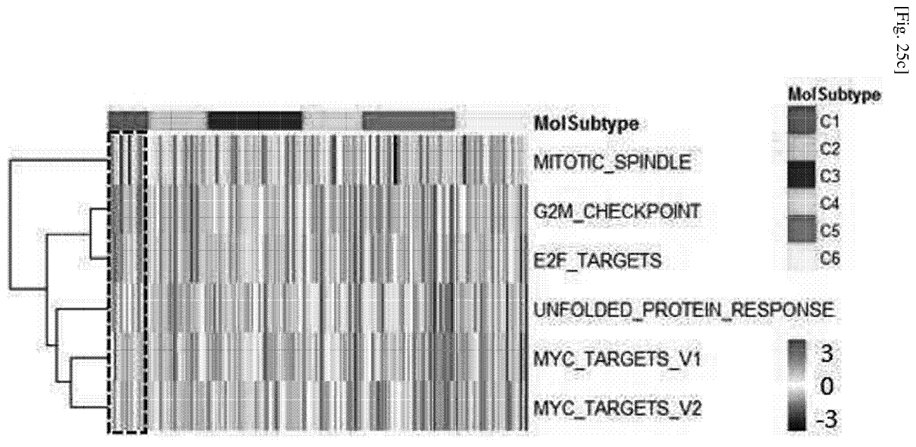

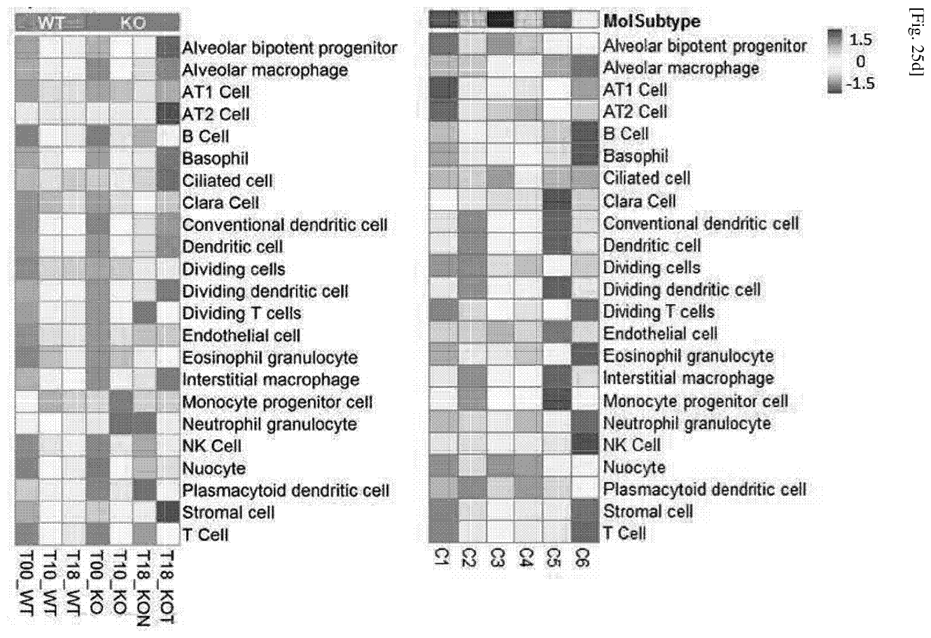

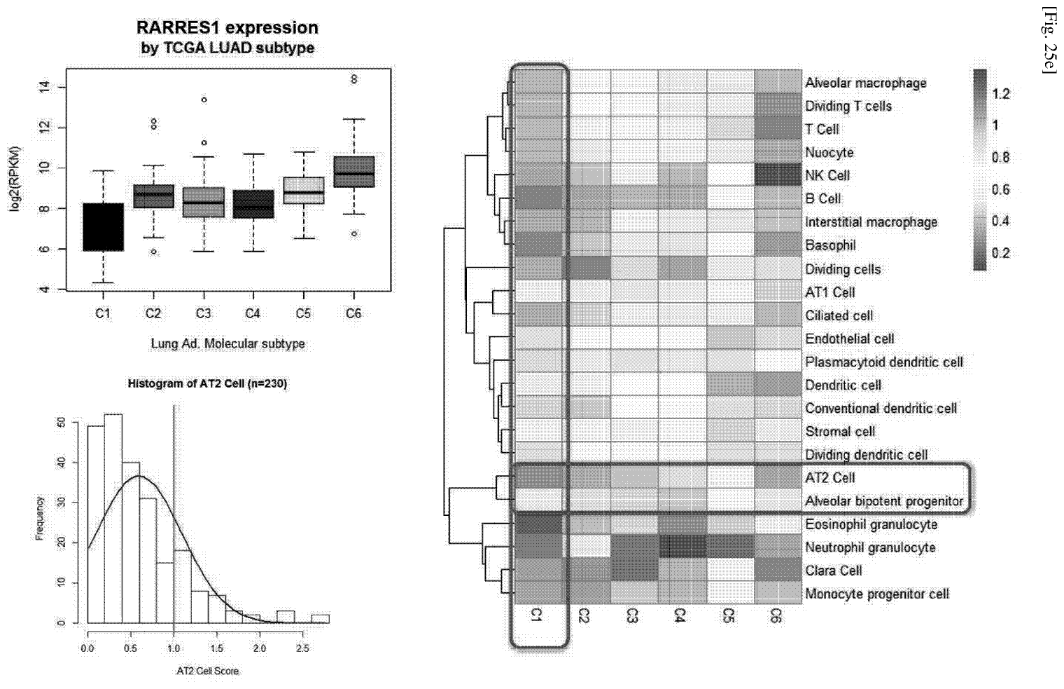

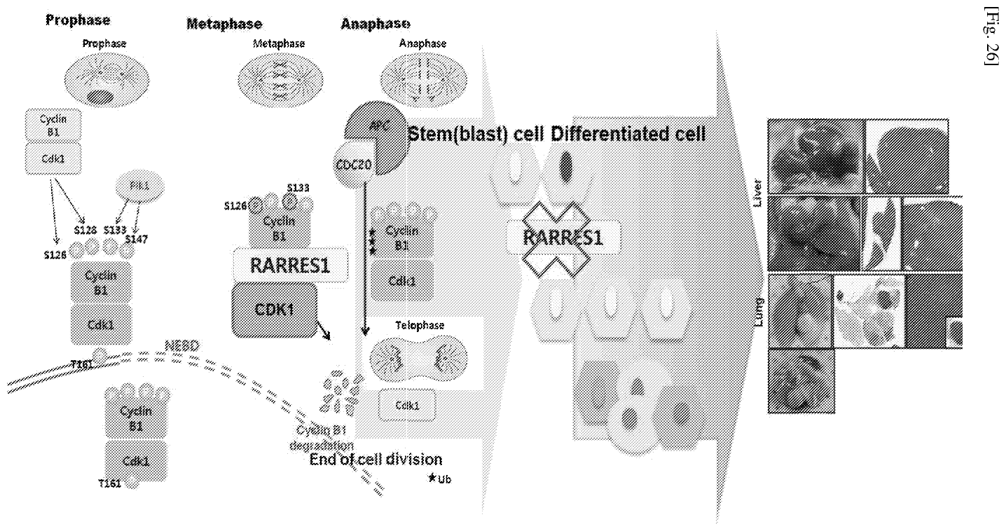

The RARRES1 state both in DNA and RNA evidence of TCGA human lung adenocarcinoma was examined (TCGA LUAD, n=230). As illustrated in FIG. 25A, for RARRES1 variants, 1.3% CNVs were amplified and there was no somatic mutation. In addition, as illustrated in FIG. 25B, there were no distinct differences in RARRES1 mRNA expression between 6 TCGA LUAD subtype clusters. Group C1 belongs to low RARRES1 expression (log 2 fold change=−1.52, T-test P-value=5.58e^{-0.8}, see FIGS. 25B and 25C).

The presence of 24 known lung cells was estimated using gene expression profiles. The proportion of most cells coincided with WT and KO states. However, alveolar type 2 (AT2) cells were extremely abundant only in KO tumor samples, but were present at a low concentration under all other conditions (fold change between KO normal and KO tumor: 2.8, see FIG. 14D). In addition, in human TCGA LUAD, group C1 exhibited a high proportion of AT2 cells (fold change: 2.7, see FIG. 25D).

In addition, as illustrated in FIG. 25E, from a graph on the left upper side, it can be seen that RARRES1 was expressed at the lowest level in group C1 among human lung cancer isoforms, it was confirmed from a graph on the right side that as in the mouse lung cancer model, when lung cells were quantitatively estimated, an AT2 cell and an alveolar bipotent progenitor were expressed at the highest levels in group C1 among human lung cancer isoforms, and it can be seen from a histogram on the left lower side that in the quantitative full distribution histogram of AT2 cells, which are lung progenitor cells, the highest level of AT2 cells was exhibited in Group C1 among human lung cancer isoforms.

The foregoing description of the present invention is provided for illustrative purposes only, and it will be understood by those of ordinary skill in the art to which the present invention pertains that the present invention may be easily modified into other particular forms without changing the technical spirit or essential characteristics of the present invention. Thus, the embodiments set forth herein should be construed as being provided for illustrative purposes only and not for purposes of limitation.

### INDUSTRIAL APPLICABILITY

RARRES1 can be widely used in screening for a cancer therapeutic agent exhibiting a decrease in a degree of binding between CDK1 and Cyclin B1, an increase in a degree of binding between the RARRES1 and CDK1 or Cyclin B1, and a decrease in an amount or activity of the CDK1 protein or the Cyclin B1 protein, and in the development of drugs. In addition, Rarres1^{-/-} animal model can be variously used for screening for a cancer therapeutic agent and developing a drug, through the relationship between RARRES1 and a CDK1-Cyclin B1 complex, the quantitative regulation of the CDK1 and Cyclin B proteins, and an increase in stem cell proliferative ability.

1. A method of screening for a cancer therapeutic agent, the method comprising the following processes:
(a) treating a sample with candidate materials in vitro;
(b) measuring a degree of binding between Cyclin-dependent kinase 1 (CDK1) and Cyclin B1 of the sample or measuring an amount or activity of a CDK1 protein or a Cyclin B1 protein; and
(c) selecting, as a cancer therapeutic agent, a candidate material exhibiting a decrease in the degree of binding between CDK1 and Cyclin B1, or a candidate material exhibiting a decrease in the amount or activity of the CDK1 protein or the Cyclin B1 protein, as compared to that in a group not treated with the candidate materials.

2. The method of claim 1, further comprising, in the process (b), measuring a degree of binding between retinoic acid receptor responder 1 (RARRES1) and CDK1 or Cyclin B1 of the sample; and,
in the process (c), selecting, as a cancer therapeutic agent, a candidate material exhibiting a decrease in the degree of binding between CDK1 and Cyclin B1 and an increase in the degree of binding between RARRES1 and CDK1 or Cyclin B1.

3. The method of claim 1, wherein, in the process (c), the decrease in the amount or activity of the CDK1 protein indicates an increase in the degradation of CDK1 in lysosomes due to an increased degree of binding between RARRES1 and CDK1.

4-5. (canceled)

6. The method of claim 1, wherein the decrease in in the degree of binding between CDK1 and Cyclin B1 indicates the inhibition of phosphorylation of serine 126 of the Cyclin B1 protein.

7. The method of claim 6, wherein the Cyclin B1 protein has an amino acid sequence of SEQ ID NO: 1.

8. The method of claim 2, wherein the increase in the degree of binding between RARRES1 and CDK1 indicates binding to inactivated CDK1 at a C-terminal portion containing amino acids 251 to 294 of the RARRES1 protein.

9. The method of claim 8, wherein the amino acids 251 to 294 of the RARRES1 protein have an amino acid sequence of SEQ ID NO: 6.

10. A method for diagnosing cancer or predicting a prognosis of cancer, the method comprising measuring a level of mRNA of retinoic acid receptor responder 1 (RARRES1) or a level of a peptide encoded by a RARRES1 gene.

11. The method of claim 10, wherein the mRNA of the RARRES1 gene has a base sequence of SEQ ID NO: 4 or 5.

12. The method of claim 10, wherein the mRNA of the RARRES1 gene comprises a nucleotide of a base sequence of SEQ ID NO: 7.

13. The method of claim 10, wherein the peptide encoded by the RARRES1 gene has an amino acid sequence of SEQ ID NO: 2 or 3.

14. The method of claim 10, wherein the peptide encoded by the RARRES1 gene comprises a peptide having an amino acid sequence of SEQ ID NO: 6.

15-17. (canceled)

18. A method of treating cancer, the method comprising: administering a pharmaceutical composition comprising an inhibitor of binding between Cyclin-dependent kinase 1 (CDK1) and Cyclin B1 as an active ingredient to an individual.

19-20. (canceled)

21. A tumorigenic Rarres1+/N chimeric animal model produced by injecting, into a blastocyst, an animal cell for producing a tumorigenic animal model, the animal cell being transfected with a retinoic acid receptor responder 1 (Rarres1) targeting vector for producing a tumorigenic animal model, the targeting vector comprising a DNA sequence consisting of, in the following order, a first locus of X-over P1 (loxP) site; a drug resistance gene region; a gene fragment comprising exon 3 of a Rarres1 genomic gene; and a second loxP site.

22. The tumorigenic Rarres1+/N chimeric animal model of claim 21, wherein the targeting vector further comprises, in front of the first locus of X-over P1 (loxP) site, a DNA sequence consisting of, in the following order, a splicing acceptor (SA), β-galactosidase (βgal), and an SV40 polyA signal (pA).

23. The tumorigenic Rarres1+/N chimeric animal model of claim 21, wherein the drug resistance gene region is a neomycin resistance gene.

24. A tumorigenic Rarres1+/− animal model produced by crossing the Rarres1+/N chimeric animal model of claim 21 with an animal expressing Cre recombinase.

25. The tumorigenic Rarres1+/− animal model of claim 24, wherein a gene encoding the Cre recombinase of the animal expressing Cre recombinase is operably linked to a zona pellucida 3 (Zp3) promoter.

26. A method of producing a tumorigenic Rarres1−/− animal model, the method comprising the following processes:
(a) producing the Rarres1+/N chimeric animal model of claim 21;
(b) producing a Rarres1+/− animal model through crossing of the chimeric animal model of process (a); and
(c) selecting a Rarres1−/− animal model from among progenies obtained by crossing the Rarres1+/− animal model of process (b).

27. (canceled)

28. The method of claim 26, wherein the Rarres1−/− animal model has a tumor induced by deletion of Rarres1.

29. (canceled)

30. A tumorigenic Rarres1−/− animal model produced by the method of claim 26.

31. The tumorigenic Rarres1−/− animal model of claim 30, wherein the animal model induces a mitotic defect or resists mitotic stress.

32. The tumorigenic Rarres1−/− animal model of claim 30, wherein the animal model induces a somatic mutation.

33. The tumorigenic Rarres1−/− animal model of claim 30, wherein in the animal model, one or more genes selected from the group consisting of Ccnd1, Cdkn1a, Cdkn2A, Nanog, Psrc1, and Nup214 are overexpressed in a mitotic cell cycle.

34. (canceled)

35. A method of screening for a tumor therapeutic agent, the method comprising the following processes:
(a) treating a sample of a tumorigenic Rarres1−/− animal model with candidate materials;
(b) measuring phosphorylation levels of Cyclin-dependent kinase 1 (CDK1) and Cyclin B1 of the sample, measuring amounts or activities of the CDK1 protein and the Cyclin B1 protein, measuring the expression or activity of muscle, intestine and stomach expression 1 (Mist1) and leucine-rich repeat-containing G-protein coupled receptor 5 (LGR5), or measuring an activity of surfactant protein C (SPC)-positive cells; and
(c) selecting, as a tumor therapeutic agent, a candidate material exhibiting a decrease in phosphorylation levels of CDK1 and Cyclin B1, a candidate material exhibiting a decrease in amounts or activities of the CDK1 protein and the Cyclin B1 protein, a candidate material exhibiting a decrease in expression or activity of Mist1 and LGR5, or a candidate material exhibiting a decrease in an activity of SPC-positive cells, as compared to that in a group not treated with the candidate materials.

36-37. (canceled)

38. The method of claim 35, wherein the Mist1, LGR5, or SPC is a stem cell marker.
